(19) United States
(12) Patent Application Publication
(10) Pub. No.: US 2009/0081638 A1
(43) Pub. Date: Mar. 26, 2009

(54) Immobilisation of Antigenic Carbohydrates to Support Detection of Pathogenic Microorganisms

(76) Inventors: Albert Anthonie Bergwerff, Amersfoort (NL); Gertruda Cornelia Antonia Maria Bokken, Beneden-Leeuwen (NL); Bertha Gerarda Maria Gortemaker, Leusden (NL)

Correspondence Address: TRASK BRITT, P.O. BOX 2550, SALT LAKE CITY, UT, 84110, US

(21) Appl. No.: 11/919,028

(22) PCT Filed: Apr. 24, 2006
(86) PCT No.: PCT/NL2006/000218

§ 371 Date: Feb. 8, 2008

(30) Foreign Application Priority Data
Apr. 22, 2005 (EP) 05075967.9

(51) Int. Cl. G01N 33/53; C07K 1/00; C12Q 1/70; C40B 50/18

(52) U.S. Cl. 435/5; 530/402; 506/32; 436/501; 435/7.1; 435/7.32

(57) ABSTRACT

The invention relates to the field of chemistry and diagnosis, more in particular to diagnosis of current and/or past and/or symptomless infections or of a history of exposure to, a gram-negative-bacterium (such as an enterobacteriaceae or a legionella). Even more in particular, the invention relates to the screening of animals or animal products for the presence of unwanted/undesired microorganisms. The invention further relates to a method for screening samples for the presence of antibodies directed against unwanted/undesired microorganisms and preferably such a method is performed with help of a biosensor. The invention also relates to a method for immobilizing polysaccharides to solid surfaces. The invention furthermore provides solid surfaces with immobilized polysaccharides as well as applications of such surfaces.

### CROSS-REFERENCE TO RELATED APPLICATION

This application is a national phase entry under 35 U.S.C. § 371 of International Patent Application PCT/NL2006/000218, filed Apr. 24, 2006, published in English as International Patent Publication WO 2006/112708 A1 on Oct. 26, 2006, which claims the benefit under 35 U.S.C. § 119 of European Patent Application Serial No. 05075967.9, filed Apr. 22, 2005.

### TECHNICAL FIELD

The invention relates to the field of chemistry and diagnosis, more in particular to diagnosis of current and/or past and/or symptomless infections or of a history of exposure to a gram-negative-bacterium (such as an enterobacteriaceae or a legionella). Even more in particular, the invention relates to the screening of animals or animal products for the presence of unwanted/undesired microorganisms. The invention further relates to a method for screening samples for the presence of antibodies directed against unwanted/undesired microorganisms and, preferably, such a method is performed with the help of a biosensor. The invention also relates to a method for immobilizing polysaccharides to solid surfaces. The invention furthermore provides solid surfaces with immobilized polysaccharides as well as applications of such surfaces.

### BACKGROUND

The world is full of gram-negative bacteria, many of which are members of the family Enterobacteriaceae. Members of this family are found in the gastrointestinal tract of animals, but many are also free living in soil and water. Members of the family Enterobacteriaceae have very complex antigenic structures. Moreover, they comprise multiple antigens that are identified as K-antigens, H-antigens and O-antigens. The K-antigen is the acidic polysaccharide capsule. The capsule has many functions including evasion from the immune system of the infected host and adhesion to the epithelium of the host. The H-antigen is located on the flagella.

The outer portion of the cell wall in gram-negative bacteria is chiefly composed of lipopolysaccharides (LPS). LPS is composed of lipid A, which is buried in the outer membrane, a short carbohydrate core and optionally a chain of polysaccharides that is made-up of repeating units. The O-antigens are located on the polysaccharide. Lipid A is the toxic constituent of the LPS. As cells lyse, LPS is released, leading to fever and complement consumption. It also interferes with coagulation and, at high concentrations, eventually leads to a state of shock.

As a non-limiting example, one member of the enterobacteriaceae, Salmonella, will be discussed in more detail. A large number of the subspecies of the genera of Salmonella enterica are important pathogenic bacteria for humans and animals. Besides animals going into a pathological episode, animals can be symptomless carriers of the bacteria. Contaminated animals can be a source of these pathogens threatening public health, for example, through the food that these animals produce. As many stakeholders consider the number of unacceptable food-borne Salmonella infections, measures have to be taken to contain this pathogen from entering the food chain.

Salmonella is of major significance as a pathogenic microorganism in food-borne infections in humans, causing mild to severe clinical effects. In The Netherlands, 5% of all identified cases of gastroenteritis are salmonellosis (Edel et al., 1993; Hoogenboom Verdegaal et al., 1994). The average incidence of this infection is 450 cases per 100,000 person years at risk, which is similar to that in other industrialized countries (Berends et al., 1998). Despite the 2480 serotypes identified in the group of S. enterica up to 2001 (Popoff, 2001), only a small number have been involved in human infections (Grimont et al., 2000). Salmonella typhimurium plus Salmonella enteritidis represented >75% of all Salmonella isolates from human sources sent to the Dutch National Salmonella Centre at the RIVM in 2002 (Van Pelt et al., 2003). This percentage consisted of 51% contributed by contact with chicken products (poultry 15%; eggs 36%) (Van Pelt et al., 2003).

Detection of immunoglobulins in the body fluids of organisms (serology) is a way to establish a history of exposure of animals and humans to infectious agents. A humoral response against Salmonella antigens can be detected in chickens one week post-infection and persists for at least ten weeks, even if the bird is no longer culture-positive (Holt, 2000). The antigenic determinants of Salmonella are, as described above, composed of somatic (O), flagellar (H) and surface (Vi) antigens (Holt, 2000). Variations in the composition of antigens correlate with different Salmonella serotypes.

Typically, serology is faster than culture-typing of the disease-causative organism. Fast and specific detection of potential Salmonella-positive herds and flocks is of importance in order to take adequate measures in production processes. The detection of antibodies in serum and blood samples from food-producing animals reporting the presence of zoonotic pathogens is, therefore, of significance. Such information is then used as the input for risk-assessment and rational slaughtering of potentially pathogen-contaminated animals in order to be able to increase food safety, but also to improve occupational hazards and to reduce spreading of the pathogens in the environment.

A number of serological tests have been developed for the detection of invasive Salmonella species. Among many such methods, agglutination and ELISA have most commonly been used (Barrow, 2000). Agglutination tests have been used successfully to eradicate Salmonella pullorum from poultry flocks. However, the approach is cumbersome, laborious and not suitable for large-scale screening programs according to modern standards. Several ELISA procedures, which are considered relatively cheap and fast, have therefore been developed to detect anti-S. enteritidis and I-antigen responses inpoultry sera (Barrow et al., 1996; Thorns et al., 1996; de Vries et al., 1998; Barrow, 2000; Yamane et al., 2000).

The use of biosensors also promises to be useful, cheap and rapid in this area of analysis. In addition, the technique is able to detect multiple analytes of any biomolecular type in a single run. A biosensor is defined as an analytical device consisting of (i) a re-usable immobilized biological ligand that “senses” the analyte, and (ii) a physical transducer, which translates this phenomenon into an electronic signal.

The surface plasmon resonance (SPR) phenomenon was first recognized in the early 1960s (Kretschmann and Raether, 1968) and the first SPR biosensors were introduced in the 1980s (Liedberg et al., 1983). It took until the late 1980s and early 1990s before the first commercially available SPR-based biosensor equipment was released on the market. Initially, this type of biosensor attracted the interest of pharmaceutical companies as a secondary tool for both selective and sensitive in vitro screening of promising novel pharmaceutical products from combinatorial libraries. It proved to be a valuable alternative for classic approaches such as ELISA procedures. Moreover, it offers real-time measurement of the binding event in contrast to end-point determinations. The benefits of this analytical approach have also been recognized by many other life science disciplines, including food sciences (Ivnitski et al., 1999; Medina, 1997). So far, only a few publications on SPR biosensing have addressed the detection of pathogenic microorganisms, for example, the use of immobilized Escherichia coli O157:H7 cells to screen the performance of anti-E. coli O157:H7 antibodies (Medina et al., 1997), and the use of these antibodies to detect E. coli O157:H7 cells (Fratamico et al., 1997).

In Jongerius-Gortemaker et al. (2002), a study to the suitability of an SPR optical biosensor to detect antibodies in serum and blood indicating a humoral reaction to invasion with Salmonella serotypes enteritidis and typhimurium was initiated. In this study, use was made of immobilized flagellar antigen fusion proteins. After thorough analysis, it was concluded that the sensitivity and/or the robustness of this system was not sufficient and, in particular, not for high-throughput screening of, for example, poultry at the slaughter line in an abattoir, processing animals at the rate of several thousands per hour.

### DISCLOSURE OF THE INVENTION

The goal of the present invention is to provide for a method that has an improved sensitivity and/or an improved robustness. This goal has been reached by developing a carrier with immobilized somatic or so-called O-antigens. As described, the O-antigens are located on the lipopolysaccharides and the composition of the polysaccharide varies and corresponds with the serovar of the Salmonella (sub)species. Every serotype can, amongst others, be described by a number of O-antigens and are typically coded with a number, such as O4, O6 or O12. The O-antigens can be found as repeating units on the polysaccharide part of the LPS. The length of the polysaccharide also varies and can be between zero (rough LPS) and more than 50 repeating units (smooth LPS).

Within the Salmonella-enterica family, different serogroups can be distinguished; each group comprises at least one specific O-antigen. The Salmonella serovars of importance in chicken and pigs are listed with their O-antigen profile in Table 1. In Denmark, Germany, Greece and The Netherlands, 39.5% of all Salmonella-positive pigs sampled at the abattoir were determined as S. typhimurium. Dependent of country, other important isolates from pigs were S. derby (17.1%), S. infantis (8.0%), S. panama (5.1%), S. ohio (4.9%), S. london (4.4%), S. livingstone (3.1%), S. virchow (2.7%), S. bredeny (2.1%), S. mbandaka (1.1%), S. Brandenburg (1.0%), S. goldcoast (0.8%).

In the case of chickens, 14% of the chickens were Salmonella-positive at flock level in 2002 in The Netherlands. The predominant serovar was in that case S. paratyphi B var. java. At the retail level, a comparable percentage (13.4%) was found in the Netherlands. The most frequent Salmonella serovars isolated from broilers in 14 EU member states were S. paratyphi B var. java (24.7%), S. enteritidis (13.6%), S. infantis (8.0%), S. virchow (6.7%), S. livingstone (5.7%), S. mbandaka (5.5%), S. typhimurium (5.3%), S. senftenberg (5.0%), S. hadar (3.7%). S. paratyphi B var. java is dominating, but this is fully attributable to The Netherlands.

TABLE 1
Some Salmonella serovars considered as important zoonotic agents in
broilers and in pigs listed with their O-antigen profiles (Popoff, 2001)

| Salmonella | Chicken (C)/ | O-antigen | |
|---|---|---|---|
| serovar | pigs (P) | profile | serogroup |
| Brandenburg | P | 4, [5], 12 | B |
| Bredeny | P | 1, 4, 12, 27 | B |
| Derby | P | 1^{a}, 4, [5]^{b}, 12 | B |
| Enteritidis | C | 1, 9, 12 | D₁ |
| Goldcoast | C/P | 6, 8 | C₂ |
| Infantis | C/P | 6, 7, 14 | C₁ |
| Livingstone | P | 6, 7, 14 | C₁ |
| London | P | 3, 10, [15] | E₁ |
| Mbandaka | P | 6, 7, 14 | C₁ |
| Meleagridis | P | 3, 10, [15], [15, 34]^{c} | E₁ |
| Ohio | P | 6, 7, 14 | C₁ |
| Panama | P | 1, 9, 12 | D₁ |
| Paratyphi B var. Java | C | 1, 4, [5], 12 | B |
| Typhimurium | C/P | 1, 4, [5], 12 | B |
| Virchow | P | 6, 7, 14 | C₁ |

^{a}O-antigen determined by phage conversion is indicated by underlining
^{b}O-antigens that may be present or absent are indicated in square brackets
^{c}lysogenized by phage ε₁₅ [15] and by phage ε₃₄ [15, 34]

In a first embodiment, the invention provides a method for immobilization of a polysaccharide on a carrier, comprising contacting the polysaccharide with an oxidizing agent and a polymer comprising at least two amine and/or amide groups to obtain a polysaccharide-polymer complex and coupling the polysaccharide-polymer complex to the carrier. The polymer can be any polymer that contains at least two amine and/or amide groups. At least two amine and/or amide groups preferably cross-link the polymer to the polysaccharide and the carrier. To allow for more efficient coupling it is preferred that the polymer comprises at least four and, more preferably, at least seven amine or amide groups. The polymer comprises at least ten building blocks. Building blocks of a polymer share characteristic reactive groups that enable elongation of the polymer. A preferred building block is an amino acid or a functional part, derivative and/or analogue thereof. In a preferred embodiment, the polymer comprises a protein. A protein comprises at least one polypeptide chain comprising at least ten amino acids or functional equivalents thereof. A protein contains at least two constituents having free amine and/or amide groups. In the context of the invention, the protein can also be a multimer comprising at least two polypeptide chains that are covalently or non-covalently linked to each other. The protein may comprise modifications such as those common to biological systems, such as post-translational glycosylation. The protein may also be artificially modified or provided with a further group as long as it has the mentioned amine and/or amide groups available.

In a preferred embodiment, the polysaccharide is derived from a gram-negative bacterium. The sensitivity of such a prepared carrier is much improved when the lipopolysaccharide (O-antigen), before the immobilization on the carrier is oxidized in the presence of a polymer, comprises at least two amine and/or amide groups, preferably a protein. Although we do not wish to be bound by any theory, it is currently thought that the aldehyde groups that result from the oxidation of the polysaccharides are capable of reacting with the amino groups of the protein to form a substituted imine (Schiff-base binding). Upon injection over an activated carrier (for example, a sensor chip), the available aldehyde groups react with hydrazide to form hydrazon. The following reduction not only stabilizes the covalent binding between the carrier (for example, a carrier comprising dextran) and the polysaccharide, but also the imine binding between protein and polysaccharide. As will be explained in more detail in the experimental part, polysaccharides (O-antigens) of different Salmonella seratypes have been immobilized on a carrier. The prepared carriers were subsequently subjected to an SPR-analysis with standard sera. The obtained serological response was used as an indicator for success of the method. When coupling reactions were performed without the oxidation step, no or almost no significant response of reference sera could be detected.

Preferably, the immobilization/coupling of the polysaccharide-protein complex to a carrier is such that high sensitivity and/or robustness is obtained. While flagellar antigens denature and lose their antigenicity towards serum antibodies and the sensor chip has to be regenerated for the next analysis cycle with relatively harsh solvents, the somatic antigens are found to be rather stable towards these regeneration solvents. In fact, the loss of immobilized O-antigen activity is believed to be primarily associated with degradation of the solid surface, namely, gradual loss of the dextran layer attached to the gold film, to which the antigens are bound. The method according to the invention results in a carrier that is more robust compared to a carrier of the prior art.

Preferably, the invention provides a method for immobilization of a polysaccharide on a carrier comprising contacting the polysaccharide with an oxidizing agent and a protein to obtain a polysaccharide-protein complex and coupling the polysaccharide-protein complex to the carrier, wherein the polysaccharide is derived from a gram-negative bacterium and, even more preferably, wherein the polysaccharide is derived from an enterobacteriaceae. Yet even more preferably, the polysaccharide is derived from a gram-negative bacterium that is a human or veterinary or plant pathogen. Examples of such polysaccharides are polysaccharides derived from a Salmonella (sub)species. Other examples are polysaccharides derived from the Escherichia coli species (for example, E. coli O157) and the bacterial species outlined in Table 2.

TABLE 2
Examples of LPS-containing bacteria pathogenic
to human and/or animals.

| Bacterial species | Mainly found in | affecting |
|---|---|---|
| Campylobacter coli | Swine | Humans |
| Campylobacter jejuni | Avian species, dogs | Humans |
| Campylobacter lari | Seagull | Humans |
| Escherichia coli O157 | Ruminants | Humans |
| Legionella pneumophila | Water | Humans |
| Listeria monocytogenes | As food process- | Humans |
| | contaminant | |
| Salmonella choleraesuis | Swine | Swine |
| Salmonella enteritidis | Avian species, swine | Humans |
| Salmonella gallinarum | Avian species | Chickens |
| Salmonella goldcoast | Swine | Humans |
| Salmonella infantis | Chickens | Humans |
| Salmonella livingstone | Swine | Humans |
| Salmonella meleagridis | Swine | Humans |
| Salmonella pullorum | Avian species | Chickens |
| Salmonella typhimurium | Avian | Humans, horses |
| Streptococcus suis | Swine | Swine, humans |
| Vibrio cholerae (non-O1) | Aquatic animals | Humans |
| Vibrio parahaemolyticus | Aquatic animals | Humans |
| Vibrio vulnificus | Aquatic animals | Humans |
| Yersinia enterocolitica | Swine | Humans |

As will be explained in more detail later, a carrier comprising an immobilized polysaccharide (O-antigen) is particularly useful in the diagnosis of the mentioned LPS-containing bacteria.

The term “polysaccharide” is intended to mean an entity comprising two or more glycoside-linked monosaccharide units and embraces, amongst others, an oligosaccharide (two to ten residues) as well as a polysaccharide (more than ten monosaccharides). The linking may result in linear or branched polysaccharide. In a preferred embodiment, the invention provides a method for immobilization of a polysaccharide on a carrier, comprising contacting the polysaccharide with an oxidizing agent and a protein to obtain a polysaccharide-protein complex and coupling the polysaccharide-protein complex to the carrier, wherein the polysaccharide is a lipopolysaccharide (LPS), i.e., a polysaccharide comprising lipid A. It is clear to a skilled person that the used (lipo)polysaccharide must comprise at least one antigenic structure and one group available/suitable for providing a linkage between the protein and the polysaccharide. More details with respect to the last item will be provided later on. Hence, as long as the (lipo)polysaccharide comprises an antigenic structure and a group suitable for providing a linkage between the protein and the polysaccharide, an immobilization method of the invention may be used to obtained a sensitive and/or robust carrier.

The LPS is expressed at the cellular exterior and is part of the bacterial cellular wall. The expression of LPS is not under direct genetic control, so that LPS is a pool of different molecules with varying composition of the lipid A part in terms of the attached aliphatic chain. Moreover, the bacterial cell may synthesize rough LPS with either a short carbohydrate chain or none at all, or smooth LPS with a mature carbohydrate chain consisting of more than 50 repeating units expressing its antigenicity. In addition to this heterogeneity, within a single LPS molecule, several O-antigen entities, which are distinctively numbered, may be expressed. An O-antigen profile is, however, per definition unique for a Salmonella serogroup. A complete serotyping of a Salmonella also includes the H-antigens as well as the Vi-antigens.

LPS may be obtained by a variety of methods and the experimental part describes in more detail the use of a trichloric acid extraction (optionally followed by ethanol extraction and dialysis) according to Staub (1965) for this purpose. Other examples of suitable extraction methods are described by Wilkons (1996) and include, but are not restricted to, extractions with diethylene glycol, dimethyl sulphoxide, NaCl-diethyl ether (1:2 (v/v)), NaCl-butan-1-ol (1:1 (v/v)), aqueous EDTA, NaCl-sodium citrate, aqueous phenol or aqueous phenol-chloroform petroleum.

The purity of the obtained/used LPS batch is not considered to be extremely critical. It has been experienced that the LPS does not have to be completely free of contaminants. The specific coupling reaction provides a certain degree of selectivity. Moreover, as described in the experimental part, the used/obtained LPS (preferably an LPS batch) is optimized with respect to the amount of protein necessary for an optimal response. It is clear to a skilled person that the LPS preferably does not comprise much rough LPS. The preferred LPS batch essentially comprises smooth LPS.

Although we do not wish to be bound by any theory, it is currently thought that the presence of a 2-keto-3-deoxy-octonic acid (KDO) and/or a glycerol-mannoheptose (Hep) and/or a GlcNAc in the core of the LPS molecule is needed for a covalent coupling.

Although a lot of different bacteria are employed by the term gram-negative bacteria, it is believed that LPS from all these bacteria are suitable for use in the presently claimed invention as long as the LPS comprises at least one constituent with non-conjugated or de-conjugated vicinal hydroxy groups, preferably in the core region of the LPS molecule. In. Salmonella, the most likely candidate constituents are KDO and Hep and GlcNAc residues. The presence or absence of such a KDO and/or Hep and/or GlcNAc group is indirectly genetically determined. Although the genetic information necessary to construct the species-, serotype- or even strain-specific monosaccharides is present in the corresponding organism, it depends on the growth circumstances whether the LPS contains aldehyde-convertible monosaccharides in the core region.

There are, of course, other sources of LPS also available, such as buying it commercially.

In a preferred embodiment, the invention provides a method for immobilization of a polysaccharide on a carrier, comprising contacting the polysaccharide with an oxidizing agent and a protein to obtain a polysaccharide-protein complex and coupling the polysaccharide-protein complex to the carrier, wherein the protein is a protein (for example, a serum protein) with a certain amount of (primary) amines. Preferably, at least some of these amines are not sterically hindered and/or are not participating in non-covalent bindings, such as H-H bridges or dipole-dipole interactions and/or are not protonated. Such a protein preferably does not have, or hardly has, any immunogenic properties and, hence, cross-reacting antibodies directed to the used protein are avoided as much as possible. Examples of suitable proteins are hemoglobin (Hb), ovalbumin (Ob), myoglobin (Mb) and serum albumin (SA). The biosensor responses of different standard sera on immobilized LPS oxidized in the presence of Hb or Ob or Mb or SA were determined. Serum albumin, myoglobin and hemoglobin gave the most promising results. In a preferred embodiment, the protein is hemoglobin or myoglobin.

The necessary protein is obtained commercially or by (overexpressing) in a suitable expression system or by isolating it from a suitable source. Hemoglobin has, for example, been obtained by isolating it from blood. Preferably, the used protein batches are as pure as possible, thereby circumventing as much cross-reaction as possible. It has been experienced, however, that small amounts of contamination are allowed without jeopardizing the sensitivity and/or robustness of the obtained carriers.

The ratio (lipo)polysaccharide versus protein depends, amongst others, on the protein used. Experiments with Hb have shown that concentrations between 15% and 50% (m/m) have resulted in satisfactory results. When the protein is bovine serum albumin, much lower concentrations, between 0.7% and 7% (m/m), are used. Some examples: the optimal Hb concentration for S. livingstone LPS is around 50% (m/m) and for S. enteritidis LPS, the optimal Hb concentration is 15% (m/m).

The isolated LPS preparations are preferably oxidized in the presence of a protein facilitated by an oxidizing agent. In a preferred embodiment, the invention provides a method for immobilization of a polysaccharide on a carrier, comprising contacting the polysaccharide with an oxidizing agent and a protein to obtain a polysaccharide-protein complex and coupling the polysaccharide-protein complex to the carrier, wherein the oxidizing agent is capable of oxidizing vicinal diols. Even more preferably, the oxidizing agent preferably oxidizes vicinal diols, at least under controlled conditions. Oxidation of vicinal diols is preferred as this warrants reliable coupling of vicinal diol-containing polysaccharide to the matrix. In a preferred embodiment of the invention, the polysaccharides to be coupled to the matrix contain an antigen that is to be recognized by a member of a binding pair. To be recognizable, it is preferred that the antigen is left unchanged, at least in the majority of the polysaccharides that are being coupled to the carrier. This requires a balance between the level of oxidation required to obtain efficient coupling to the matrix and availability of the antigen for association with the member of the binding pair. The latter requires that the antigen is left essentially unaffected by the oxidation, at least in an amount sufficient to be usable in a diagnostic setting. Oxidation of vicinal diols according to the present invention warrants the availability of sufficient antigen in recognizable form, while at the same time allowing efficient coupling of the polysaccharide to the carrier. In a preferred embodiment, the oxidizing agent comprises (sodium) m-periodate. Other periodates, such as potassium periodate or other salts thereof, are also suitable periodates of the present invention.

Periodate oxidation is very suited for enabling preferential oxidation of vicinal diols according to the present invention. Oxidation of predominantly vicinal diols in a polysaccharide of the invention can typically be achieved by incubating the polysaccharide with the periodate at a concentration of between 1 and 10-mM periodate. Other parameters of the reaction influence both the speed and the type of reaction predominantly performed. One example is incubation time. When applying very short incubation times, higher than 10 mM periodate can be used.

Periodate preferably oxidizes vicinal diols, particularly of the more susceptible vicinal diols in the side chains of the polysaccharide. Thus, as long as so-called “mild” reaction conditions are chosen, preferably, vicinal diols will be oxidized. When conditions are chosen that also allow other oxidation reactions to occur more often (for instance, because of depletion of the vicinal diol substrate), the antigen present in the polysaccharide will be affected significantly. Thus, for the present invention, a periodate oxidation is said to be mild when the mentioned preferred concentrations are used and when at least 20%, preferably at, least 50%, more preferably at least 70%, and most preferably about 90% of the antigen is intact after oxidation. Availability or intactness of the antigen is preferably measured by means of an ELISA assay using a standardized antibody. Again, we do not wish to be bound by any theory, but it is currently thought that periodate will induce an oxidative disruption of linkages between vicinal diols especially on carbohydrate moieties, as in, e.g., mannose, to yield aldehyde functionalities. This reaction is typically performed in buffers at a pH range between 4.5 and 5.5 in the dark using a (preferably) freshly prepared 1 to 100 mM sodium meta-periodate in 0.1 M sodium acetate. Preferably, the reaction is performed at a concentration of between 1 and 10 mM metaperiodate. The oxidation is performed in the presence of a protein in the ranges as discussed above.

The bis-aldehyde compounds, like the oxidized monosaccharide constituents in the polysaccharide chain of LPS, may react with any amino group in a protein and may form a Schiff-base linkage resulting in a substituted imine. When one or both of the vicinal hydroxyl groups is condensed in a covalent sugar linkage, the hydroxyl function is lost and no oxidation occurs. This is the case in many branched and/or linearly linked oligo- and polysaccharides. In the case of Salmonella LPS, the inner core structure carries in most cases an oxidizable Gal, GlcNAc, Hep and/or KDO, but non-reducing Hep and KDO constituents are most susceptible for oxidation, in particular, at very mild oxidation conditions at concentrations less than 6 mM meta periodate. Because the core region is a rather conserved part of LPS from different Enterobacteriaceae, (lipo)polysaccharides of members of the Enterobacteriaceae, may be applied in a method of the invention.

Periodate will also oxidize, when present, certain aminoethanol derivatives, such as the hydroxylysine residues in collagen, as well as methionine (to its sulfoxide) and certain thiols (usually to disulfides). In addition, N-terminal serine and threonine residues of peptides and proteins can be selectively oxidized by periodate to aldehyde groups. These reactions, however, usually occur at a slower rate than oxidation of vicinal diols and the presence of such groups does not substantially interfere with a method according to the invention.

The invention also provides a method for immobilization of a polysaccharide on a carrier, comprising contacting the polysaccharide with an oxidizing agent and a protein to obtain a polysaccharide-protein complex and coupling the polysaccharide-protein complex to the carrier, further comprising a step that results in ending/stopping the oxidation process, for example, by desalting of the polysaccharide-protein complex. This is, for example, accomplished with the help of a NAP-5 column. However, the person skilled in the art is aware that many other methods exist that have the same effect, for example, adding a reductor or an easily oxidizable molecule, such as glycerol. Preferably, the way of stopping the oxidation is that a buffer change is accomplished at the same time, for example, HPLC, FPLC, dialysis, ion-exchangers, gel electrophoresis or ultrafiltration.

For storage purposes, the production of evaporated aliquots, after addition of protein, is also described within the experimental part. This results in the presence of a large stock of reproducible material.

The invention, therefore, further comprises the obtained intermediate, i.e., the preparation, in the presence of protein-oxidized polysaccharide, optionally desalted and optionally evaporated.

Preferably, the used carrier is made of an inert, non-hydrophobic material and the binding of the LPS-protein complex to the carrier is covalent. Even more preferred, the carrier has a low protein binding or low biomolecular binding, for example, a carrier of glass or silica or of a non-hydrophobic plastic. In a preferred embodiment, the carrier is in the form of a microsphere or bead. Several types of a microsphere or beads are available to the person skilled in the art. In a preferred embodiment, the microsphere or bead comprises polystyrene. A microsphere or beads are particularly preferred because they can be provided with different antigens using a method of the invention. A microsphere or beads with different antigens can be accordingly coded with a different color. Testing a sample for the presence of an antibody against an antigen can be done using a collection of the mentioned microsphere or beads. Binding of the antibody to a particular type of antigen can now be detected easily by the color code of the microsphere or bead bound. Binding of the antibody can be detected in various ways. For instance, a microsphere or beads containing bound antibody can be extracted from the sample and measured using a further antibody specific for the constant region of the antibody. On the other hand, samples can be directly analyzed, i.e., in the absence of further manipulations by labeling the bound antibody and simultaneously detecting the color of the antibody and the color of the microsphere or bead. Various methods for simultaneous detection of two or more colors are available to the person skilled in the art. In the present invention, a “color” is defined as any type of electromagnetic radiation that can be detected, be it a typical color revealed, for instance, by reflection of light, to light emitted as a result of fluorescence or phosphorescence.

The invention thus further provides a collection of at least two microspheres or beads wherein at least two of at least two microsphere or beads each comprise a different antigen of the present invention. In a preferred embodiment, the antigen comprises O-antigen of Salmonella. In a particularly preferred embodiment, the antigen is linked to the microsphere or beads carrier using a method of the invention. Thus, preferably, at least one of the microsphere or beads comprises a polysaccharide coating linked to a polysaccharide comprising an antigen to be detected linked to each other via a polymer comprising at least two amine and/or amide groups, preferably a protein of the invention, wherein the linkage polymer (protein) is linked to the polysaccharide comprising the antigen, via an amine and/or amide group on the polymer and a periodate-oxidized vicinal diol on the polysaccharide comprising the antigen.

In a preferred embodiment, the invention provides a method for immobilization of a polysaccharide on a carrier comprising contacting the polysaccharide with an oxidizing agent and a protein to obtain a polysaccharide-protein complex and coupling the polysaccharide-protein complex to the carrier, further comprising activating the surface of the carrier. In an even more preferred embodiment, the carrier comprises a glass surface coated with gold and even more preferred, the carrier is modified with a carboxyl donor. A surface can be activated. Carboxylic acid (COOH) groups (further referred to as carboxyl groups) are needed on this surface. Preferably, these COOH groups are provided by a stable homogeneous layer of molecules, which may have been modified for this purpose. These surfaces may exist of, but are not limited to, carboxylic acid-modified polysaccharides, alkanes or alkenes, such as polyethylene, attached to, e.g., gold, polystyrene or silicon surfaces. Preferably, the carrier comprises a polysaccharide that acts as a carboxyl donor; more preferably, a carboxymethylated dextran layer, wherein the polysaccharide carrier preferably comprises a dextran layer activated with 1-ethyl-3-(3-dimethylaminopropyl)carbodiimide hydrochloride and N-hydroxysuccinimide. The activation is preferably followed by preparation with carbohydrazine. In the next step, the polysaccharide-protein complex is added to the activated dextran layer. The reactive aldehyde functionalities react spontaneously with the hydrazide to hydrazones, which are then reduced to stabilize the covalent bonds.

Prior to routine use, the performance of chip-conjugated LPS to bind anti-Enterobacterium (for example, Salmonella) antibodies is assessed using reference polyclonal agglutination sera.

Depending on the analytical/diagnostic question asked, it is decided whether one or, for example, at least two different serogroup-representing carbohydrates, preferably four different serogroup-representing carbohydrates, are used. In case one is interested in knowing which particular serogroup is present, multiple (the amount of which is different on the particular question asked and on the used apparatus) different serogroup-representing carbohydrates are used and if one just wants to know whether, for example, an animal is or has been infected by a particular serogroup, a single serogroup-representing carbohydrate may be oxidized in the presence of a protein and immobilized on a carrier. The use of at least two different serogroup-representing carbohydrates results in a carrier that can be used in a multi-serogroup analysis. More, preferably at least three and, even more preferred, at least more than three (for example, four) different polysaccharides are used. These polysaccharides may be oxidized in the presence of one type of protein or in the presence of different types of protein. The skilled person is capable of making any sensible combination. For example, to be able to detect more than 90% of all Salmonella infections, serogroups B, C and D in chickens and serogroups B, C, D and E in pigs should be represented.

Using one type of serogroup-representing carbohydrates is extremely useful if one is interested in the question whether or not a certain type of bacterium is or was present. Using multiple different serogroup-representing carbohydrates is, for example, useful if one wants to determine whether an animal is or was infected by any gram-negative bacteria (for example, enterobacteriaceae).

In a preferred embodiment, the invention provides a method for immobilization of a polysaccharide on a carrier, comprising contacting the polysaccharide with an oxidizing agent and a protein to obtain a polysaccharide-protein complex and coupling the polysaccharide-protein complex to the carrier, wherein the carrier is a biosensor chip. Such a biosensor chip is commercially available (for example, that produced by Biacore) and, hence, no further information will be provided.

In another embodiment, the invention provides a carrier obtained by the method as described above or a carrier comprising an immobilized polysaccharide-protein complex on its surface. In one embodiment of the invention, a carrier of the invention comprises a polysaccharide coating that is linked to a further polysaccharide coating via reductive amination, wherein the further polysaccharide coating comprises a protein coupled to the further polysaccharide coating through oxidation of vicinal diols on the further polysaccharide protein. In a preferred embodiment, the reductive amination is achieved. In a preferred embodiment, the invention provides a carrier comprising a polysaccharide coating that is coupled to a polysaccharide.

In yet another embodiment, the invention provides a biosensor comprising a carrier according to the invention. Whether the carrier is obtained by a method according to the invention can, for example, be determined by extracting the polysaccharides from the carrier and determining whether covalently linked protein is present. As already discussed above, the carrier may also comprise different immobilized polysaccharides (for example O-antigens) possibly in combinations with different types of protein. However, also one type of protein may be used in the oxidation of different polysaccharides.

Whether a carrier and/or chip of the invention is employed can, for example, be determined with the help of MALDI-MS, possibly in combination with proteolytic digestion. Such an analysis provides information with respect to the used protein and polysaccharide. With help of acidic hydrolysis, the polysaccharide-protein complexes are released from the carrier. Such an obtained mixture is then subjected to LC-MS/MS analysis before and after proteolytic hydrolysis. The obtained complex may also be subjected to a monosaccharide analysis, for example, GC-MS following methanolysis and/or Smith degradation, from which it is determined which type of LPS is used. This information is furthermore used to determine whether KDO, Hep or other sugars have been oxidized.

A carrier of the invention may be used in different detection systems, for example, optical, thermal, acoustic, amperometric, magnetic or chemical, and a carrier of the invention may be used in any biomolecular interaction assay (BIA) or any affinity assay (AA). As a non-limiting example, the use of optical detection via Surface Plasmon Resonance is described in more detail.

The invention provides a Surface Plasmon Resonance detection system comprising a biosensor as described above. The gold layer in the sensor chip creates the physical conditions required for Surface Plasmon Resonance (SPR). The principle of SPR will be described in the context of Biacore instruments. They incorporate the SPR phenomenon to monitor biomolecular interactions in “real-time.” At an interface between two transparent media of different refractive index, such as glass and water, light coming from the side of the higher refractive index is partly reflected and partly refracted. Above a certain critical angle of incidence, no light is refracted across the interface and total internal reflection (TIR) occurs at the metal film-liquid interface. This is where light travels through an optically dense medium, such as glass, and is reflected back through that medium at the interface with a less optically dense medium, such as a buffer.

Although the incident light is totally reflected, the electromagnetic field component, termed the evanescent wave, penetrates a distance on the order of one wavelength into the less optically dense medium. The evanescent wave is generated at the interface between a glass prism (high refractive index) and a layer of buffer (lower refractive index). If the interface between the media of higher and lower refractive indices is coated with a thin metal film (a fraction of the light wavelength), then the propagation of the evanescent wave will interact with the electrons on the metal layer. Metals contain electron clouds at their surface, which can couple with incident light at certain angles. These electrons are also known as plasmons, and the passage of the evanescent wave through the metal layer causes the plasmons to resonate, forming a quantum mechanical wave known as a surface plasmon. Therefore, when surface plasmon resonance occurs, energy from the incident light is lost to the metal film resulting in a decrease in the reflected light intensity.

The resonance phenomenon only occurs at an acutely defined angle of the incident light. This angle is dependent on the refractive index of the medium close to the metal-film surface. Changes in the refractive index of the buffer solution (e.g., an increase in surface concentration of solutes), to a distance of about 300 nm from the metal film surface will, therefore, alter the resonance angle. Continuous monitoring of this resonance angle allows the quantitation of changes in refractive index of the buffer solution close to the metal-film surface. In “real-time”. Biacore, the metal film properties, wavelength, and refractive index of the glass (denser medium) are all kept constant and, as a result, SPR can be used to monitor the refractive index of the aqueous layer immediately adjacent to the metal (gold) layer.

In the Biacore system, the chip is composed of glass, has four channels and the associated gold layer is covered with a layer of dextran chemically modified to facilitate immobilization of ligands, such as antibodies or antigens. Any changes in mass that occur due to binding of the analyte with the immobilized antibody on the sensor chip will cause a change in SPR angle, which is monitored in “real-time” and quantified as a sensorgram. A mass change of approximately 1 kRU (1,000 RU) corresponds to a mass change in surface protein concentration of 1 ng/mm². Typical responses for surface binding of proteins are of the order of 0.1 to 20 kRU.

There is no need to label molecules with fluorescent or radioactive tags, thereby avoiding the possibility that labels may compromise activity and, moreover, avoiding difficult or expensive chemistry necessary for labeling.

The obtained carriers can be used in different types of analysis, such as bacteriology (direct assay) or serology (indirect assay).

An example of a serological assay is a method for determining the presence of an antibody directed to an antigen of a gram-negative bacteria in a sample, comprising contacting the sample with a carrier or a biosensor as described above and determining whether the carrier has bound any antibody (FIG. 1).

Such a method is, for example, very suitable for determining the presence of an antibody directed against an O-antigen and, thus, it is indirectly established whether an infection is present or whether a recent infection has occurred. Such a method is, for example, used to screen slaughter animals for Salmonella or to screen animals for Salmonella before they are exported abroad. Moreover, the method is also applied to samples obtained from living (for example, farm or zoo) animals.

Examples of samples that can be used in such a method are tissue samples, body fluid, secretes or excretes and more detailed examples are blood, blood-derived samples, tissue, meat juice, milk, egg, fluids from an eye, saliva or feces. As already outlined, the samples can be obtained from dead as well as living animals.

A method according to the invention is not limited to a certain immunoglobulin (sub)type but can, in principle, be every (iso)type immunoglobin, such as (s)IgA₁, (s)IgA₂, IgD, IgG₁, IgG₂, IgG₃, IgG₄, IgM, and IgY. Moreover, it may also be any other antigen-binding material. Preferably, such an antigen-binding material is a biomarker of a (history) of infection.

Such a serological assay is, for example, directed to one particular serogroup-representing carbohydrate or to different (i.e., multi analyte) serogroup-representing carbohydrates and, hence, such a method is, for example, used to determine the presence or absence of a certain Salmonella (sub)type.

An example of a bacteriological assay is a method for determining the presence of a gram-negative bacterium in a sample comprising contacting the sample with a predetermined amount of antibodies directed against an antigen of the bacterium and determining the amount of antibodies not bound to the bacterium with a carrier or a biosensor as described above.

Preferably, the antigen is a serogroup-representing carbohydrate.

Optionally, this method further comprises the removal of non-bound antibodies from the contacted sample and a predetermined amount of antibodies by, for example, washing or immuno-magnetic separation procedures, centrifugation or filtering.

For this type of analysis, every type of sample can be used, such as animal feed, manure, feathers, soil, water for consumption or sewage water, meat, orange juice, chocolate, skin, vegetables, etc. Animal samples may be obtained from living as well as dead animals.

In this bacteriological assay, a single type of antibody, as well as a mixture of at least two different types of antibodies (directed against different antigens, for example, two different serogroup-representing carbohydrates), is used.

Preferably, such serological and bacteriological assays are performed such that the binding to the carrier or the biosensor is determined by Plasmon Surface Resonance or fluorescent microsphere or bead counter.

The source of the samples is, as already outlined above, unlimited and may be obtained, for example, from a human or an animal. Examples of suitable animals are (race) horses, pigs, poultry (for example, chicken, turkey, quail, duck, and goose), ruminants (for example, calf or cow, goat, and sheep). The animals may be farm animals or zoo animals, as well as free-living animals. Moreover, samples from these animals may be obtained from living as well as dead animals.

In yet another embodiment, the invention provides a method for determining the presence of a gram-negative bacterium in a sample comprising: contacting the sample with target bacteria-specific bacteriophages and allowing the bacteriophages to infect the sample; removing non-bound and/or non-invading bacteriophages resulting in a bacteriophage-infected sample; bringing the bacteriophage-infected sample into contact with an indicator organism susceptible for the used bacteriophages; incubate during at least one bacteriophage multiplication cycle; recover the bacteriophages to obtain a bacteriophage-containing sample; and analyze the bacteriophage-containing sample with a carrier or a biosensor as described above.

Most analytical methods require prior enrichment and growth in specific media to detect bacteria, including Salmonella. Usually, sample preparation is very time consuming, relative to the total analysis time. It generally takes three to five days before the presence of, e.g., Salmonella, can be confirmed. In many situations, this time for analysis is unacceptable and hinders trade and indirectly threatens community health.

The objective of this part of the invention is development of a fast (preferably within 24 hours), cost-effective, specific, and/or sensitive diagnostic method for the determination of the presence of microorganisms. For this reason, the development of a biomolecular interaction assay (BIA) that exploits the ability of genus- and/or serovar-specific bacteriophages to multiply in their “victim” bacteria is aimed. An increment in number of the target pathogen-specific phage(s) not only indicates the presence of the target organism but is also a (semi-)quantitative measure for the content of target bacteria in the tested sample.

A schematic overview of the proposed BIA method is depicted in FIG. 2. A particulate sample is homogenized, for example, using a Stomacher. Liquid samples are mixed by vigorous shaking. Analyte cells are then extracted or enriched by any suitable method and may comprise (a combination of) selective growth, centrifugation, filtration and/or immuno-magnetic separation (IMS). Enriched cells are fortified with target bacteria-specific bacteriophages and incubated for a few minutes while mixing. Before the multiplication cycle of the bacteriophage is complete, cells are washed to remove, as complete as possible, any non-bound and non-invading bacteriophages. Following the multiplication cycle of the bacteriophage, the sample is brought in contact with an indicator organism susceptible, i.e., in a life phase that is sensitive for bacteriophage penetration and intracellular multiplication, for the used bacteriophage, preferably at the highest possible concentration (for example, concentrated overnight culture). The bacteriophage-bacterium suspension is incubated for at least one bacteriophage multiplication cycle. The phage-infected suspension is then centrifuged or filtered to precipitate/remove cellular material and to recover multiplied bacteriophages. The bacteriophage-containing sample is injected over an LPS-conjugated biosensor chip (according to the invention) to retain these particles in the detector for the generation of analyte-specific biosensor response.

To gain as much time as possible, the indicator organism can be kept as a continuous culture in the lab and has a cell density of usually 10⁹ CFU/ml. Such a suspension may be concentrated to 10¹⁰ CFU/ml, as higher cell densities will increase sensitivity of the proposed method.

This method can be used to determine a single type of serovar but in order to detect multiple serovars in one run, a mix of different bacteriophages and a mixture of possibly different indicator bacteria may have to be applied.

Target bacteria-specific bacteriophages are described in the prior art and examples are provided in the experimental part, for example, anti-Salmonella enteritidis bacteriophages.

Phages have been described to attach to LPS, including the phage described in the experimental part for Salmonella detection. Suitable carriers/chips are carriers/chips with LPS or with immobilized bacterial surface molecules (thus including membrane proteins and other biomolecules or a combination thereof). Use of LPS of cell membrane material will circumvent the generation of poly- or monoclonal antibodies. If attachment of the phages to bacterial biomolecules (LPS) is not satisfactory in the BIA, biosensor chip-immobilized anti-phage antibodies may have to be used in a successful BIA to capture bacteriophages from the probed sample.

The invention, furthermore, provides a kit with components suitable for use in any of the described applications. Depending on the customer's demand, such a kit comprises a ready-for-use carrier/chip obtained by a method according to the invention. When the customer wants to prepare the carrier himself, the kit will at least comprise (lipo)polysaccharide fortified/enriched with protein (for example, hemoglobin or serum albumin) in a predetermined amount, an amount of oxidizing agent (for example, periodate), and suitable buffers. Optionally, such a kit comprises means for desalting, for example, a desalting column. When the customer wants to mix (lipo)polysaccharide and protein himself, these components are delivered separately, together with an instruction manual. Optionally, such a kit may, furthermore, comprise positive and/or negative reference sera, a sample dilution buffer and any necessary instruction manual.

The methods as described above are particularly suitable for screening samples on a large-scale basis. In one of the earlier (slow) settings, 96 samples were checked within 33 minutes. In a large-scale setting with relatively slow biosensor equipment, 15,000 samples were screened within three months. This number could have been much higher but, unfortunately, one of the slaughterhouses stopped participating.

The invention will be explained in more detail in the following description, which is not limiting to the invention.

### BRIEF DESCRIPTION OF THE DRAWINGS

FIG. 1: Schematic representation of one embodiment of the method according to the invention.

FIG. 2: Schematic outline of the BIA for detection of bacteria using bacteriophages as indicator organisms. Indicator organisms may be cultured overnight or shorter.

FIG. 3: Reactivity of Hb-fortified, oxidized LPS isolated from S. typhimurium (batch St2003.2) with agglutination sera in relative arbitrary biosensor responses (RU). The Hb fortification level of LPS during oxidation is depicted in the figure. The expected binding of the agglutination sera is listed in Table 3.

FIG. 4: ROC curves from Example 2, Experiment 1. TPF: True-positive fraction; FPF: false-positive fraction.

FIG. 5: ROC curves from Example 2, Experiment 2. TPF: True-positive fraction; FPF: false-positive fraction.

FIG. 6: Analysis of prepared beads coated with LPS from S. enteritidis (reflecting serogroup D), S. goldcoast (reflecting serogroup C₂), S. livingstone (reflecting serogroup C₁), S. meleagridis (reflecting serogroup E) and S. typhimurium (reflecting serogroup B). The success of the coating and specificity of the LPS were tested with commercially available monoclonal antisera against O4 (serogroup B), O5 (seregroup B), O7 (serogroup C₁), O8 (serogroup C₂) and O9 (serogroup D). Responses are expressed in arbitrary units as median fluorescence index (MFI) at the Y-axis, whereas the X-axis indicates the type of LPS conjugation of the individual beads.

FIG. 7: Analysis of prepared beads coated with LPS reflecting serogroups B, C₁, C₂ and D. The activity of the coating was tested with monoclonal antisera against O4 (serogroup B), O5 (serogroup B), O7 (serogroup C₁), O8 (serogroup C₂) and O9 (serogroup D). Similar to FIG. 6, except zoomed in on the lower responses. Notice that response of anti-O5 is under broken. For details, see legend of FIG. 6.

FIG. 8: Comparison of beads coated with two different oxidation batches of oxidized LPS from S. enteritidis (reflecting serogroup D) and S. goldcoast (reflecting serogroup C₂), S. livingstone (reflecting serogroup C₁) and S. typhimurium (reflecting serogroup B). The coating was tested with commercially available monoclonal antisera against O5 (serogroup B), O7 (serogroup C₁), O8 (serogroup C₂) and O9 (serogroup D).

FIG. 9: Analysis of meat drip and serum from chickens. Commercially available antisera were used to spike meat drip and serum. Drip, liquid extract collected from muscle tissue from a chicken, which was tested as Salmonella-free using standard ISO methods; Drip+CH-SPF, drip, that was spiked with serum collected from specific pathogen-free (SPF) chickens; CH-SPF, serum obtained from specific pathogen-free (SPF) chickens; DripSPA-PG, drip that was spiked with antiserum reactive with S. pullorum and S. gallinarum; SPA-PG, anti-S. pullorum and anti-S. gallinarum antiserum; DripCHSi, drip that was spiked with chicken serum that was serologically positive for a S. infantis infection; CH-Si, chicken serum serologically positive for S. infantis. The X-axis indicate the type of LPS conjugation of the individual beads. See FIGS. 6, 7 and 8 for more details.

FIG. 10: Analysis of swine sera spiked with commercially available anti-S. typhimurium (yellow-colored bars) and anti-S. livingstone (cyan-colored bars). In addition, beads in buffer solution (blue-colored bars) and negative swine serum (purple-colored bars) were analyzed on beads that were coated with LPS representing serogroups B, C₁, C₂ and D.

FIG. 11: Binding of bacteriophage FO1 to immobilized LPS from S. typhimurium, S. enteritidis, S. goldcoast and S. livingstone on a Biacore SPR biosensor. PFU, plaque-forming units.

FIG. 12: Binding of bacteriophage FO1 to an SPR biosensor chip coated with S. typhimurium-LPS following incubation of S. typhimurium, S. enteritidis, S. goldcoast, S. livingstone with 1.2×10⁹ PFU bacteriophage FO1. Dotted line indicates the cut off value.

FIG. 13: Incubation of different food pathogens and spoilage bacteria in the presence of Salmonella spp.-specific bacteriophage FO1. During growth, the optical density at λ 600 nm as a measure of bacterial growth was monitored. bl+FO1, blank medium devoid of bacteria supplemented with bacteriophages exclusively.

FIG. 14: Incubation of different Salmonella serovars in the presence of Salmonella spp.-specific bacteriophage FO1. For more details, see legend of FIG. 13.

FIG. 15: Incubation of different food pathogens and spoilage bacteria in the presence of Salmonella spp.-specific bacteriophage FO1. The number of plaque-forming units (PFU) was determined following an incubation of five hours. bl+FO1, blank medium-devoid of bacteria supplemented with bacteriophages exclusively.

FIG. 16: Incubation of different Salmonella-serovars in the presence of Salmonella spp.-specific bacteriophage FO1. For more details, see legend of FIG. 13. FO1 stock was not incubated.

FIG. 17: SPR biosensor-analysis of bacteriophage FO1 propagated in different Salmonella serovars after concentration and dialysis of the viruses. The suspensions were serial diluted and analyzed; the final concentrations of concentrated/diluted bacteriophages is indicated at the X-axis.

FIG. 18: Oxidation of carbohydrate moiety. R′ and R indicate the distal and the proximal positions, respectively, in the carbohydrate chain.

FIG. 19: Conjugation to a polyamine-containing molecule (R″), such as a protein.

FIG. 20: Immobilization to fluorescent beads and stabilization of chemical bonds.

FIG. 21: Schematic representation of the procedure of LPS coupling to beads and analysis of serum.

FIG. 22a: Examples of Campylobacter-infecting bacteriophage.

FIG. 22b: Examples of Listeria-infecting bacteriophages.

FIG. 22c: Examples of Salmonella-infecting bacteriophages.

FIG. 22c: Examples of Salmonella-infecting bacteriophages.

FIG. 23: Counting chamber and technique.

FIG. 24: Total area of a Bürker-Türk counting chamber (A) of 1 mm², in which B represents the area of 1/16^{th} of the total area.

FIG. 25: Effect of periodate concentration on the immobilization of LPS of S. enteritidis on a SPR biosensor chip.

FIG. 26: Effect of periodate concentration on the antigenic activity of immobilized LPS of S. enteritidis (batch Se2002.1). LPS was immobilized to a biosensor chip and analyzed in a SPR biosensor (FIG. 25). Range tested was 0.2 mM to 1.8 mM sodium periodate. O9, O12, O poly A-S, S. typhimurium, SE, biosensor response from anti-O9 antisera; anti-O12 antisera, polyclonal antibody against serogroups A to S, chicken serum positive for S. typhimurium and chicken serum positive for S. enteritidis, respectively.

FIG. 27: Effect of periodate concentration on the antigenic activity of immobilized LPS of S. enteritidis (batch Se2002.1). Range tested was 1.8 mM to 48.6 mM sodium periodate. For more details, see FIG. 26.

FIG. 28: Effect of periodate concentration on the immobilization of LPS of S. goldcoast on a SPR biosensor chip.

FIG. 29: Effect of periodate concentration on the antigenic activity of immobilized LPS of S. goldcoast (batch Sg2002.1). LPS was immobilized to a biosensor chip and analyzed in a SPR biosensor (FIG. 28). Range tested was 0.2 mM to 5.4 mM sodium periodate. O6,7, O8, O-poly A-S, S. livingstone, S. infantis, biosensor response from anti-O6/7 antisera; anti-O8 antisera, polyclonal antibody against serogroups A to S, porcine serum positive for S. livingstone and chicken serum positive for S. infantis, respectively.

FIG. 30: Effect of periodate concentration on the immobilization of LPS of S. Livingstone on a SPR biosensor chip.

FIG. 31: Effect of periodate concentration on the antigenic activity of immobilized LPS of S. goldcoast (batch Sg2002.1). LPS was immobilized to a biosensor chip and analyzed in a SPR biosensor (FIG. 30). Range tested was 0.2 mM to 5.4 mM sodium periodate. O6,7, O8, O-poly A-S, S. livingstone, S. infantis, biosensor response from anti-O6/7 antisera; anti-O8 antisera, polyclonal antibody against serogroups A to S, porcine serum positive for S. Livingstone and chicken serum positive for S. infantis, respectively.

FIG. 32: Schematic presentation of the procedure.

FIG. 33: Quality sheet for LPS extraction process.

FIG. 34: Immobilization of LPS to biosensor surface and stabilization of chemical bonds.

FIG. 35: Biacore 3000 control software on COM 1.

FIG. 36: Immobilization wizard.

FIG. 37: Immobilization test wizard.

FIG. 38: Logging.

FIG. 39: Typical sensorgram of immobilization of oxidized LPS. Report points: 1 baseline; 2 activating EDC/NHS; 3 carbohydrazide; 4 ethanolamine; 5 Immobilization LPS Se.

FIG. 40: Sensorgram of anti Salmonella O-Poly A-S analyzed on a LPS-containing CM5 chip.

FIG. 41: Typical serological responses of agglutination sera and group-specific Salmonella antisera on a S. goldcoast LPS—hemoglobin-immobilized CM5 biosensor chip.

FIG. 42: Typical serological responses of avian reference sera (SPF-CH, EIA St, EIA Se, SPA-PG and CH-Si sera) and swine reference sera (SW-sera) on a S. goldcoast LPS—hemoglobin-immobilized CM5 biosensor chip.

FIG. 43: Typical baseline responses of a S. goldcoast LPS—hemoglobin-immobilized CM5 biosensor chip.

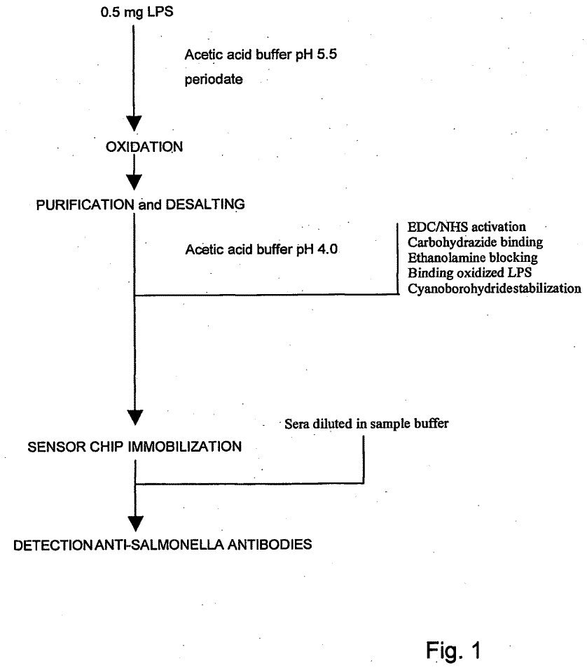

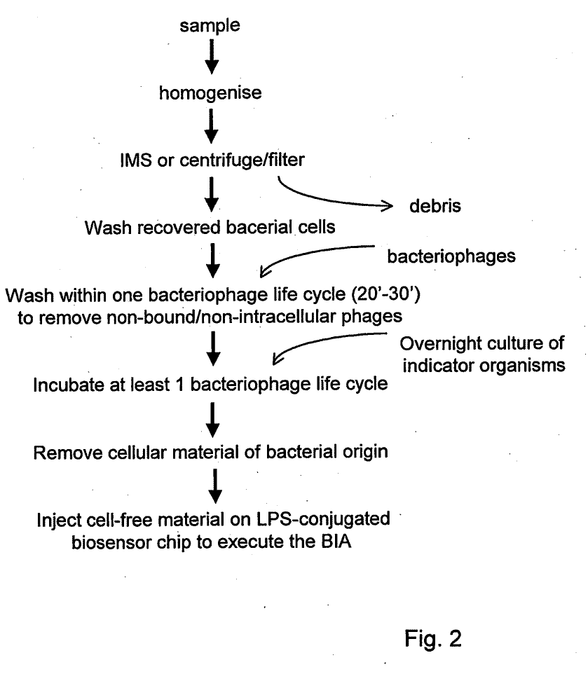

### DETAILED DESCRIPTION OF THE INVENTION

### Examples

### Example 1

### 1. Materials and Methods

1.1 Materials

1.1.1 Chemicals

Amine-coupling kits, consisting of N-hydroxysuccinimide (NHS), 1-ethyl-3-(3-dimethylaminopropyl)carbodimide hydrochloride (EDC) and ethanolamine hydrochloride—sodium hydroxide pH 8.5 and the running buffer (HBS-EP), containing 10 mM HEPES, 150 mM sodium hydrochloride, 3 mM EDTA and 0.005% (v/v) surfactant P20 at pH 7.4, were bought from Biacore AB (Uppsala, Sweden), which also supplied ready-to-use 10 mM glycine and 50 mM sodium hydroxide. Ethanol, ethylene glycol, sodium chloride, sodium hydroxide and trichloroacetic acid (TCA) were purchased from Merck (Darmstadt, Germany). Carboxymethylated-dextran sodium salt, sodium, cyanoborohydride and carbohydrazide were obtained from Fluka Chemie GmbH (Buchs, Switzerland). CHAPS (Plus one) was delivered by Pharmacia Biotech (Uppsala, Sweden). Sodium acetate trihydrate and acetic acid were supplied by J. T. Baker (Deventer, The Netherlands). Guanidine hydrochloride was obtained from Calbiochem (San Diego, Calif., USA). Porcine hemoglobin (Hb) and myoglobin (Mb), chicken ovalbumin (Ob; 98% grade V), bovine serum albumin (BSA; 96% Fraction V), sodium periodate, TWEEN®20, TWEEN®80 and Triton X-100 were acquired from Sigma Chemical Company (St. Louis, Mo., USA). Water was obtained from of a MILLI-Q® water purification system-(Millipore, Bedford, Mass., USA).

1.1.2 Materials

NAP-5 columns (0.5 ml; Sephadex G-25) were purchased from Amersham Biosciences (Roosendaal, The Netherlands) and were used as described by the producer. CM5 biosensor chips were bought from Biacore AB. Dialysis bag (Spectra/Por) with a cut-off of 1 kDa was obtained from Spectrum Laboratories Inc. (Rancho Dominguez, Calif., USA).

1.1.3 Anti-Salmonella Antisera

The following Salmonella monovalent “O” somatic lapine antisera were used: anti-O4, anti-O5, anti-O6,7, anti-O8, anti-O9, anti-O10, anti-O12, and O-PolyE (anti-O3, anti-O10, anti-O15, anti-O19, and anti-O34). In addition, Salmonella polyvalent “O” somatic (Poly A-S) rapine antisera (anti-O2, anti-O3, anti-O4, anti-O5, anti-O6,7, anti-O8, anti-O9, anti-O10, anti-O1, anti-O12, anti-O13, anti-O15, anti-O16, anti-O17, anti-O18, anti-O19, anti-O20, anti-O21, anti-O22, anti-O23, anti-O28, anti-O30, anti-O34, anti-O35, anti-O38, anti-O40, and anti-O41) was used as well. The sera were purchased from Pro-Lab Diagnostics (Salmonella Reference Section of the Central Veterinary Laboratory, Weybridge, U.K.). Serogroup-specific murine anti-B (anti-O4, O5 and O27), anti-C (anti-O7, O8), anti-D anti-O9, O6) and anti-E (anti-O3, O19) monoclonal antibodies were bought from SIFIN (Berlin, Germany).

Sera were diluted 1:20 (v/v) in HBS-EP containing 1.0 M sodium chloride, 1% (m/v) carboxymethylated dextran and 0.05% (v/v) TWEEN®80, except anti-O5 serum was diluted 1:200 (v/v) and the anti-serogroup-specific preparations were diluted 1:100 (v/v) in the same solvent.

1.1.4 Reference Avian and Porcine Sera

All reference sera were obtained from the Dutch Animal Health Service (Deventer, The Netherlands). The obtained avian reference sera were reactive with Salmonella enteritidis (serogroup D₁), S. typhimurium (serogroup B), S. pullorum/gallinarum (serogroup D₁) and S. infantis (serogroup C₁), and were further referred to as C-Se, C-St, C-Spg and C-Si, respectively. These chicken sera were originally prepared for ELISA analyses as positive references. In addition, specific pathogen-free chicken serum (further referred to as C-SPF) was purchased as a negative control reference sample. These sera were reconstituted from freeze-dried material by addition of water at a volume indicated by the manufacturer. C-Se, C-Spg and C-Si were diluted 1:200 (v/v) in HBS-EP containing 1.0 M sodium chloride, 1.0% (m/v) carboxymethylated dextran and 0.05% (v/v) TWEEN®80, whereas C-SPF and C-St were diluted 1:50 (v/v) in the same solution. Likewise, porcine sera from animals challenged with S. typhimurium and S. livingstone (serogroup C₁) were referenced as P-St and P-Sl, respectively. In addition, Actinobacillus pleuropneumoniae serotype 2-reacting porcine serum used as control in a complement fixation test, was exploited as negative control for porcine serum in the Salmonella biosensor assay. The porcine sera were diluted 1:20 (v/v) in HBS-EP containing 1.0 M sodium chloride, 1% (m/v) carboxymethylated dextran and 0.05% (v/v) TWEEN®80 as end concentrations.

1.1.5 Salmonella Stock

The bacteria Salmonella goldcoast (Sg; serogroup C₂), S. livingstone (Sl) and S. meleagridis (Sm; serogroup E₁) were obtained from an in-house collection, while S. enteritidis #23 phage type Pt4 (Se), and S. typhimurium X-193 phage type 507 (St) were kind gifts of F. G. van Zijderveld (Animal Sciences Group, Lelystad, The Netherlands). The bacteria were grown in overnight cultures in Nutrient Broth #2 (Oxoid, Basingstroke, U.K.). Stocks of Salmonella strains were morphologically and biochemically confirmed as Salmonella and also verified for the presence of the correct, expected O-antigens by an agglutination reaction of the cells with specific standard anti-O-antigen antisera (Pro-Lab Diagnostics) as indicated in Table 3 on a glass plate. After addition of a half of the original volume with glycerol (Merck), stocks were stored in portions at −80° C.

TABLE 3
O-antigen verification of the Salmonella serovars used for LPS production.
The expected reaction of antisera used for verification by agglutination is given.

| Salmonella | Agglutination sera | | | | | | | | |
|---|---|---|---|---|---|---|---|---|---|
| Serovar | α-O4^{a} | α-O5 | α-O6, 7 | α-O8 | α-O9 | α-O10 | α-O12 | poly A-S | Poly E |
| S. enteritidis | − | − | − | − | + | − | + | + | − |
| (O9, O12)^{b} | | | | | | | | | |
| S. goldcoast | − | − | + | + | − | − | − | + | − |
| (O6, 8) | | | | | | | | | |
| S. livingstone | − | − | + | − | − | − | − | + | − |
| (O6, 7) | | | | | | | | | |
| S. meleagridis | − | − | − | − | − | + | − | + | + |
| (O3, O10) | | | | | | | | | |
| S. typhimurium | + | + | − | − | − | − | + | + | − |
| (O4, O5, O12) | | | | | | | | | |

^{a}α-O4: antibodies reacting with antigen structure coded with O4; in a similar way, the antibodies against O5, O6, 7, O9, O10 and O12 are indicated.
^{b}O-antigens specific for Salmonella serovar is indicated in brackets.

1.2 Methods

1.2.1 Extraction of LPS

Overnight culturesof Salmonella were prepared by applying 100 μl from their corresponding stocks on each of the 120 plates containing brain heart infusion agar (BHIa, Oxoid). The presence of the expected Salmonella serovar was confirmed through conventional selective growth, bio- and immunochemical classification, whenever new stock suspensions were produced. The bacteria were harvested from the surface of the plates into 1 ml 9 g/l NaCl (saline) solution per agar plate using a trigalski spatula. Each plate was washed twice with 2 ml saline solution. Bacteria were collected in six centrifugation tubes. Each tube was complemented with 100 ml saline and mixed before centrifugation at 10,000 g and 4° C. for 15 minutes and supernatant was discarded. This centrifugation step was repeated twice by suspending cells in 75 ml saline wash solution per tube each run.

While kept on ice, pelleted bacteria were suspended in water at a volume ratio, which was five-fold to the weight of the bacteria. An equivolume of 0.250 M (Se) or 0.500 M (Sg, Sl, Sm and St) TCA was added to give end concentrations of 0.12 M and 0.25 M, respectively, followed by continuous stirring at 4° C. for 3 hours. A lipopolysaccharide (LPS)-containing supernatant was then acquired at 20,000 g and 4° C. for 30 minutes. The pH of the supernatant was adjusted to pH 6.5 with 5 M sodium hydroxide and, when nearing the aimed pH, with 0.10 M sodium hydroxide. The final volume of the LPS-containing solution was determined prior to storage at −18° C. for 30 minutes. The solution was diluted with a double volume of freezing cold absolute ethanol from a −18° C. storage place, and incubation was continued overnight at −4° C. without stirring in a closed, in-house-built device with circulating cold ethylene glycol/water (1:4, v/v). An LPS-containing pellet was obtained after centrifugation at 20,000 g and −4° C. for 30 minutes. The particulate material was suspended in a volume of 0.5 ml water per gram original bacterial mass weighed at the start of the extraction process. The suspension was dialyzed in a 1-kDa dialysis bag against water at 4° C. for two days with regular intermittent refreshment of the water. The bag content was centrifuged at 20,000 g and at 4° C. for 30 minutes, and the supernatant was lyophilized. The lyophilisate was weighed to establish the recovery of LPS. LPS was reconstituted in water to make up an end concentration of 5 mg/ml. Dependent of type of LPS and batch (see also section 1.2.2), a volume of 1 mg/ml porcine hemoglobin (Hb) was added to a concentration as indicated in the text. Each batch was portioned into 0.5-mg LPS fractions, which were dried using a vacuum evaporator and then stored at 5° C. to 8° C.

1.2.2 Optimal Hemoglobin Content

Protein was added to an LPS preparation prior to its chemical modification and immobilization to a sensor chip to acquire high coating levels and high serum-responsive antigens. The optimum Hb content in each LPS batch was established by comparison of the responses of immobilized LPS that was fortified with Hb at different levels, using a panel of positive and negative reference sera.

1.2.3 Oxidation of LPS

A portion of 0.5 mg hemoglobin-fortified PS was dissolved in 500 μl 100 mM sodium acetate pH 5.5. Following the addition of 20 μl 50 mM sodium periodate, the solution was incubated for 40 minutes on ice protected from light. The oxidation of LPS was quenched and the solution was desalted by passing 500 μl of the reaction mixture through an NAP-5 cartridge with a gravity-controlled flow. Modified LPS was eluted with 1 ml 10 mM sodium acetate, pH 4.0. Prior to use, the cartridge was conditioned thrice with 3 ml 10 mM sodium acetate, pH 4.0.

1.2.4 Immobilization of LPS

To immobilize the antigens to a sensor chip, the following handlings were conducted at a flow rate of 5 μl/minutes in a Biacore 3000 instrument controlled by Biacore 3000 Control Software (version 3.1.1; Biacore). Immobilization of oxidized LPS was achieved by execution of the aldehyde-coupling procedure described in BIA Applications Handbook, version AB (1998). Briefly, the dextran layer at the biosensor chip CM5 was activated with a seven-minute pulse of a mixture of EDC/NHS available from the amine-coupling kit. The activation was immediately followed by injection of 5 mM aqueous carbohydrazide for seven minutes as well.

Deactivation of the excess of reactive groups was then accomplished with a pulse of 1 M ethanolamine for seven minutes. Prior to immobilization of the antigen, LPS was diluted in sodium acetate pH 4.0 in a ratio dependent of the Salmonella serovar (see text) and immobilized for 32 minutes. The linkage between dextran-matrix and antigen was then stabilized by injection of 100 mM sodium cyanoborohydride dissolved in 10 mM sodium acetate at pH 4 at a flow rate of 2 μl/minute for 20 minutes. A relative response indicative for a successful LPS immobilization procedure is 2 kRU for a 62.5 μg/ml LPS solution containing 15% (m/m) protein, and 9 kRU for a 250 μg/ml LPS solution containing 50% (m/m) protein.

1.2.5 SPR Biosensor Assay

Optical SPR biosensor assays were performed on a Biacore 3000 SPR biosensor platform controlled by the same software as described above. Prior to injection, sera were diluted in HBS-EP buffer containing 1.0% (m/v) carboxymethylated-dextran sodium salt, 1.0 M sodium chloride and 0.05% (m/v) TWEEN®80 at a ratio of 1:50 (v/v) or otherwise as indicated in the text. The mixtures were incubated for at least two minutes at ambient temperature. Pig sera were injected for two minutes at 40 μl/minute, whereas bird sera were injected for two minutes at 5 μl/minute or 20 μl/minute as indicated.

Regeneration of the chip to recover the antigenic activity of the sensor surface was achieved with a 15-second pulse of 6 mM glycine at pH 2, containing 6 M guanidine hydrochloride, 0.1% (m/v) CHAPS, and 0.1% (v/v) of each TWEEN®20, TWEEN®80 and Triton X-100. This was followed with a second regeneration step with the running HBS-EP buffer enriched with 0.05% (m/v) CHAPS (end concentration) for 12 seconds at 100 μl/minute.

1.2.6 Monosaccharide Analysis

Trimethylsilylated, (methyl ester) methyl glycosides were prepared from the glycan samples by methanolysis (1.0 M methanolic HCl, 24 hours, 85° C.) followed by re-N-acetylation and trimethylsilylation, and then analyzed by gas chromatography/mass spectrometry as described (J. P. Kamerling and J. F. G. Vliegenthari (1989)). The quantitative analysis was carried out by gas chromatography on a capillary EC-1 column (30 m×0.32 mm, Alltech) using a Chrompack CP 9002 gas chromatograph operated with a temperature program from 140° C. to 240° C. at 4° C./minute, and flame-ionization detection. The identification of the monosaccharide derivatives was confirmed by gas chromatography/mass spectrometry on a Fisons Instruments GC 8060/MD 800 system (Interscience) equipped with an AT-1 column (30 ml×0.25 mm, Alltech).

### 2. Results

2.1. LPS Isolation

For the production of LPS, yields of bacterial cells and of LPS were compared for agar plate culture and growth of Salmonella in broth (Table 4). For laboratory technical reasons, it was decided to harvest bacteria from agar plates, rather than isolation of the cells from culture flasks. The results of the isolation of LPS from Se, Sg, Sl, Sm and St are summarized in Tables 5 to 9, respectively. The standardized isolation of well-defined LPS is determinative for a successful and robust serological assay. To secure assay performance, batch-to-batch differences should be kept to a minimum. For this reason, several batches of LPS extracted from each Se, Sg, Sl, Sm and St were produced. The recovery of LPS largely depended on the final TCA concentration in the mixture during extraction of LPS (cf. Table 6 and Table 8), although this relationship was not completely clear for the extraction of LPS from St (Table 9). Indeed, no accurate optimal TCA concentration could be determined for each LPS type through the testing of a broad range of TCA concentrations. Here, optimal TCA would yield the highest IFS amounts and give the highest specific serological and lowest aspecific biosensor responses. In this study, the TCA concentration chosen as “optimal” for LPS extraction from the different Salmonella serotypes was based on the final LPS yields after dialysis, and were 0.12 M; 0.25 M, 0.25 M, 0.25 M and 0.25 M as end concentrations for Se, Sg, Sl, Sm and St, respectively.

TABLE 4
Recovery of LPS from S. enteritidis cells grown either as a suspension
in a bioreactor containing so-called nutrient broth#2 (broth) or on BHI
agar plates (agar). LPS was isolated using indicated TCA end
concentrations. The yield of LPS relative to the amount of
isolated cells is indicated in the last column.

| LPS | | | | | |
|---|---|---|---|---|---|
| Batch | Culture | | bacteria yield | recovered | LPS yield |
| code | method | TCA (M) | (g) | LPS (mg) | (%, m/m) |
| Se01 | Broth | 0.25 | 3.9 | 16 | 0.42 |
| Se02 | Broth | 0.25 | 5.0 | 21 | 0.41 |
| Se03* | Agar | 0.25 | 14 | 0.2 | 0.00 |
| Se04a | Broth | 0.25 | 3.4 | 0.4 | 0.01 |
| Se04b | broth | 0.5 | 3.4 | 2 | 0.06 |
| Se05** | agar | 0.5 | 7.6 | 2.4 | 0.03 |
| Se06a | agar | 0.5 | 8.8 | 3.9 | 0.04 |
| Se06b | agar | 0.25 | 7.6 | 9.5 | 0.12 |
| Se06c | agar | 0.125 | 8.9 | 13 | 0.14 |
| Se07a | agar | 0.1 | 9.1 | 12 | 0.13 |
| Se07b | agar | 0.05 | 9.2 | 2.9 | 0.03 |
| Se07c | agar | 0.025 | 9.3 | 4 | 0.04 |
| Se2003.1 | agar | 0.125 | 29 | 48 | 0.16 |
| Se2003.2 | agar | 0.125 | 17 | 25 | 0.15 |
| Se2003.4 | agar | 0.125 | 13 | 5.1 | 0.04 |
| Se2005.1 | agar | 0.25 | 15 | 18 | 0.13 |

*pH of TCA-containing mixture is outlying
**some material was lost during sample work-up process.

TABLE 5
Recovery of Salmonella enteritidis cells grown on BHIa plates.
LPS was isolated using 0.12 M TCA end concentration
(cf. Table 4).

| LPS | Total | | bacteria | |
|---|---|---|---|---|
| Batch | bacterial | Number of | per plate | Rec. LPS/cells |
| (Se) | yield (g) | BHIa plates | (g) | (% m/m) |
| Se2003.1 | 29.09 | 98 | 0.29 | 0.16 |
| Se2003.2 | 17.27 | 60 | 0.28 | 0.15 |
| Se2003.4 | 13.00 | 40 | 0.32 | 0.04 |
| Se2005.1 | 44.5 | 120 | 0.37 | 0.12 |

Rec, recovered.

TABLE 6
Recovery of Salmonella goldcoast cells grown on BHIa plates. LPS
was extracted using a TCA end concentration as indicated. Rec, recovered.

| LPS | Total | Number of | bacteria | | Rec. |
|---|---|---|---|---|---|
| Batch | bacterial | BHIa | per plate | TCA^{a} | LPS/cells |
| code | yield (g) | plates | (g) | (M) | (% m/m) |
| Sg2003.1 | 40.39 | 120 | 0.34 | 0.075 | 0.01 |
| Sg2003.2 | 35.09 | 120 | 0.29 | 0.25 | 0.41 |
| Sg2003.3 | 12.89 | 40 | 0.32 | 0.25 | 0.36 |
| Sg2005.1 | 46.99 | 120 | 0.39 | 0.25 | 0.30^{b} |

^{a}end concentration TCA in extraction mixture.
^{b}approximately a third of the production was lost during work-up.

TABLE 7
Recovery of Salmonella livingstone cells grown on BHIa plates. LPS
was extracted using 0.250 M TCA end concentration.

| LPS | Total | Number of | bacteria | |
|---|---|---|---|---|
| Batch | bacterial | RHIa | per plate | Rec. LPS/cells |
| code | yield (g) | plates | (g) | (% m/m) |
| Sl2003.1 | 32.89 | 120 | 0.27 | 0.52 |
| Sl2003.2 | 13.63 | 40 | 0.34 | 0.51 |
| Sl2005.1 | 47.40 | 120 | 0.40 | 0.64 |

Rec, recovered.

TABLE 8
Recovery of Salmonella meleagridis cells grown on BHIa plates. LPS
was isolated using a TCA end concentration as indicated. Rec, recovered.

| LPS | Total | Number of | bacteria | | Rec. |
|---|---|---|---|---|---|
| Batch | bacterial | BHIa | per plate | TCA^{a} | LPS/cells |
| code | yield (g) | plates | (g) | (M) | (% m/m) |
| Sm2003.1a^{b} | 9.10 | 30 | 0.30 | 0.250 | 0.32 |
| Sm2003.1b^{b} | 10.13 | 30 | 0.34 | 0.125 | 0.19 |
| Sm2003.1c^{b} | 10.00 | 30 | 0.33 | 0.075 | 0.06 |
| Sm2003.2^{b} | 40.42 | 120 | 0.33 | 0.075 | 0.02 |
| Sm2003.3 | 37.28 | 138 | 0.27 | 0.250 | 0.47 |

^{a}end concentration TCA in extraction mixture.
^{b}batches Sm2003.1 to 2003.2 were combined to a single batch called Sm2003.1

TABLE 9
Recovery of Salmonella typhimurium cells grown on BHIa plates.
LPS was isolated using 0.250 M TCA end concentration. Rec, recovered.

| | Total | Number of | bacteria | | Rec. |
|---|---|---|---|---|---|
| Batch | bacterial | BHIa | per plate | TCA^{a} | LPS/cells |
| code | yield (g) | plates^{a} | (g) | (M) | (% m/m) |
| St2003.1 | 28.51 | 69 | 0.41 | 0.125 | 0.18 |
| St2003.2^{b} | 39.21 | 120 | 0.33 | 0.250 | 0.06 |
| St2003.3^{b} | 16.6 | 56 | 0.30 | 0.125 | 0.09 |
| St2003.4a^{b} | 18.15 | 60 | 0.30 | 0.250 | 0.18 |
| St2003.4b^{b} | 19.2 | 60 | 0.32 | 0.125 | 0.06 |
| St2005.1 | 46.80 | 120 | 0.39 | 0.250 | 0.19 |

^{a}end concentration TCA in extraction mixture;
^{b}batches St2003.2 to St2003.4b were combined to a single batch called St2003.2

The monosaccharide composition of isolated LPS preparations were analyzed to reveal the consistency of the isolation and purification procedure for LPS from different Salmonella growths. It must be noted that analyses were performed on LPS preparations that were ready for oxidation and, for that reason, fortified with Hb at levels that were determined most optimal for the LPS batch tested (see below). For this purpose, GC-FID and GC-MS analyses were carried out after methanolysis of the Hb-fortified LPS preparations (Table 10 through Table 14). These results show that Hb does not contribute to a significant amount of carbohydrates in the final LPS preparation. Analysis of BHIa showed the exclusive presence of galactose (Gal) and glucose (Glc). The content of these monosaccharides was 5.6 μg/mg dried. BHIa. Analyses of the Salmonella LPS preparations demonstrated the occurrence of Gal, Glc, N-acetyl glucosamine (GlcNAc), glycero-manno-heptose (Hep), 2-keto-3-deoxy-octonic acid (KDO), mannose (Man) and rhamnose (Rha; 6-deoxy-mannose) in accordance with their carbohydrate structures. Their relative occurrence was expressed as a molar ratio relative to 1.0 Man (as part of the PS region) or relative to 3.0 Hep (as part of the core region). It should, however, be noted that the core region contains two or three Hep residues.

Furthermore, GlcNAc can originate from either GlcNAc as in the repeating unit of Sl LPS, or from glucosamine (GlcN), which occurs as disaccharide in the lipid A moiety as backbone for the attached lipids. Gal occurs in the core region, which is conserved in all Salmonella enterica serovars and in the PS region of Se, Sg, St and Sm as well. In these cases, the molar ratio of Gal is expected to be in excess of 1.0 Man, except for Sg in which each repeating unit contains two Man residues. The monosaccharide analyses did not include the detection of O-acetylated, phosphoryl-ethanolaminated or phosphorylated constituents, nor that of abequose (Abe; 3,6-dideoxy-xylohexose) or tyvelose (Tyv; 3,6-dideoxy-arabinose), which occur in the polysaccharide and core regions of the isolated LPS types as well.

Analysis of Se LPS showed the occurrence of Gal, Man and Rha at a molar ratio of 1.4, 1.0 and 1.2, respectively in batch Se2003.1, whereas this ratio was 1.1, 1.0 and 0.9, respectively, in batch Se2003.4 (Table 5). This ratio is in good compliance with the composition of a repeating unit as [Tyv-]Man-Rha-Gal, except the Rha ratio was significantly too high in batch Se2003.1. The carbohydrate content calculated on the basis of determined monosaccharides was significantly higher in batch Se2003.4, namely 241 μg compared to 123 μg of batch Se2003.1. Considering the occurrence of two GlcN residues in the lipid A and a single GlcNAc residue in the core region and a single Man residue in each repeating unit, the number of repeating units was estimated at 19 and 20 in batches Se2003.1 and Se2003.4, respectively.

Monosaccharide analysis of oxidized Se2003.1 clearly demonstrates significant differences with the non-oxidized identical batch (Table 10). In contrast to the two GlcN residues, it is expected that the non-reducing, terminal GlcNAc residue is for the greater part oxidized. Alditol derivatives were not detected by the monosaccharide analysis applied and a corresponding amount of GlcNAc-ol was not determined. As Gal and Man in the repeating unit are not susceptible towards periodate oxidation, the molar ratio of Man in the oxidized batch is normalized to that of Man in the non-oxidized batch. It should be noted that both Gal residues in the core region are susceptible towards oxidation and thus the total Gal ratio is affected. Inspection of the molecular structure of Se LPS suggests that in addition to terminal GlcNAc and core Gal; terminal KDOII-KDOIII disaccharide, conjugated HepI and terminal HepIII are susceptible to periodate oxidation as well. Indeed, the molar ratios of these monosaccharide residues suggest the loss of one Hep residue and approximately 1.6 KDO residues. It should be noted that KDOIII may not be completely oxidized when this residue is conjugated with a phosphoryl-ethanolamine group.

TABLE 10
Monosaccharide analysis of S. enteritidis LPS and of oxidized
S. enteritidis LPS. LPS was fortified with Hb at 15% (m/m). Molar ratios
were determined on the basis of two GlcN and one GlcNAc residues
(detected as three GlcNAc residues) present in the core and lipid A
regions (referred to as CORE) and on the basis of one Man residue in
the repeating unit (referred to as UNIT). Normalized GlcNAc and Man
residues are indicated by underlining. Carbohydrate content was
determined in 0.5 mg LPS preparations, except monosaccharide
analysis was performed on 125 mg oxidized material.

| | Molar ratio | | | | | |
|---|---|---|---|---|---|---|
| | | | Batch Se2003.1 | | | |
| | Batch Se2003.1 | | (oxidized) | | Batch Se2003.4 | |
| Monosaccharide | CORE | UNIT | CORE | UNIT | CORE | UNIT |
| Gal | 29.7 | 1.4 | 28.7 | 1.3 | 26.3 | 1.1 |
| Glc | 6.3 | 0.3 | 7.5 | 0.4 | 6.9 | 0.3 |
| GlcNAc | 3.0 | + | 2.2 | + | 3.0 | + |
| Hep | 2.8 | + | 1.9 | + | 2.0 | + |
| KDO | 3.2 | + | 1.4 | + | 2.3 | + |
| Man | 21.6 | 1.0 | 21.6 | 1.0 | 23.5 | 1.0 |
| Rha | 24.8 | 1.2 | 24.4 | 1.1 | 20.9 | 0.9 |

| | | | |
|---|---|---|---|
| Carbohydrate | 123.0 | —^{b} | 241 |
| content (μg)^{a} | | | |
| Nr of repeating | 19 | | 20 |
| units | | | |

^{a}does not include the contribution of Tyv residues;
^{b}Amount of LPS-containing material analyzed was not accurately determined.

Compared to Se LPS, monosaccharide analysis of Sg LPS suggests that LPS structures were smaller as the number of repeating units was significantly lower (Table 11). The relative contribution of core Gal to PS Gal is, for that reason, larger and total molar ratio is found at 1.5. In a similar way, the molar ratio for Glc is found at 1.3 (batch Sg2003.2) and 1.5 (batch Sg2003.3), whereas the molar ratio for Rha fits with the expected structure. Batch Sg2003.2 seems, however, to contain less terminal HepIII and terminal K_DOIII and could, therefore, offer less possibility for immobilization to the sensor chip.

TABLE 11
Monosaccharide analysis of LPS isolated from S. goldcoast fortified
with Hb at 50% (m/m). Molar ratios were determined on the basis
of two GlcN and one GlcNAc residues (detected as three GlcNAc
residues) present in the core and lipid A regions (referred to as
CORE) and on the basis of two Man residues in the repeating unit
(referred to as UNIT). Normalized GlcNAc and Man residues
are indicated by underlining. Carbohydrate content was
determined in 0.5 mg LPS preparations.

| Molar ratio | |
|---|---|
| Batch | Batch |
| Sg2003.2 | Sg2003.3 |

| Monosaccharide | CORE | UNIT | CORE | UNIT |
|---|---|---|---|---|
| Gal | 15.3 | 1.5 | 14.8 | 1.5 |
| Glc | 13.5 | 1.3 | 14.3 | 1.5 |
| GlcNAc | 3.0 | + | 3.0 | + |
| Hep | 2.9 | + | 2.3 | + |
| KDO | 3.0 | + | 2.8 | + |
| Man | 20.5 | 2.0 | 19.2 | 2.0 |
| Rha | 10.6 | 1.0 | 10.2 | 1.0 |

| | | |
|---|---|---|
| Carbohydrate content | 170 | 199 |
| (μg)^{a} | | |
| Nr of repeating units | 9 | 8 |

^{a}does not include the contribution of Abe residues.

Normalization of the number of core residues from the monosaccharide analysis results of Sl LPS (Table 12) was hampered by the occurrence of GlcNAc in the repeating units of the PS. When the number of Hep residues was set at 3.0, an unacceptable overestimation of the number of KDO residues arose. For that reason, the number of Hep was set at 2.0, but may need to be modified so that the number of KDO is closer to 3.0. As the number of Man residues in, each repeating unit is four, molar ratios were corrected for 4.0 Man residues. On the basis of a molar ratio of 4:1 of Man/GlcNAc in the PS region, the number of repeating units was calculated on the basis of the remaining core GlcNAc and Lipid A GlcN residues. This calculation revealed that the number of repeating units, in Sl was also relatively small, namely eight and ten units in batch S12003.1 and batch S12003.2, respectively.

TABLE 12
Monosaccharide analysis of LPS isolated from S. livingstone
fortified with Hb at 50% (m/m). Molar ratios were determined on
the basis of two Hep residues present in the core (referred to
as CORE) and on the basis of four Man residues in the repeating
unit (referred to as UNIT). Normalized GlcNAc and Man residues
are indicated by underlining. Carbohydrate content was determined
in 0.5 mg LPS preparations.

| Molar ratio | |
|---|---|
| Batch Sl2003.1 | Batch Sl2003.2 |

| Monosaccharide | CORE | UNIT | CORE | UNIT |
|---|---|---|---|---|
| Gal | 3.5 | 0.4 | 5.1 | 0.4 |
| Glc | 17.4 | 1.7 | 23.5 | 1.6 |
| GlcNAc | 14.0 | 1.4 | 18.7 | 1.3 |
| Hep | 2.0 | 0.2 | 2.0 | 0.14 |
| KDO | 2.7 | 0.3 | 2.7 | 0.2 |
| Man | 40 | 4.0 | 57 | 4.0 |
| Rha | n.d. | n.d. | n.d. | n.d. |

| | | |
|---|---|---|
| Carbohydrate content | 212 | 239 |
| (μg) | | |
| Nr of repeating units | 8 | 10 |

n.d., not detected.

TABLE 13
Monosaccharide analysis of S. meleagridis LPS. Batches Sm2003.1
and Sm2003.3 were fortified with 50% (m/m) Hb. Molar ratios were
determined on the basis of two GlcN and one GlcNAc residues
(detected as three GlcNAc residues) present in the core and
lipid A regions (referred to as CORE) and on the basis of one
Man residue in the repeating unit (referred to as UNIT). Normalized
GlcNAc and Man residues are indicated by underlining.
Carbohydrate content was determined in 0.5 mg LPS preparations.

| Molar ratio | |
|---|---|
| Batch | Batch |
| Sm2003.1 | Sm2003.3 |

| Monosaccharide | CORE | UNIT | CORE | UNIT |
|---|---|---|---|---|
| Gal | 20.4 | 1.5 | 22.4 | 1.4 |
| Glc | 7.9 | 0.6 | 4.8 | 0.3 |
| GlcNAc | 3.0 | + | 3.0 | + |
| Hep | 2.8 | + | 2.9 | + |
| KDO | 2.8 | + | 2.9 | + |
| Man | 14.2 | 1.0 | 15.4 | 1.0 |
| Rha | 16.1 | 1.1 | 17.6 | 1.2 |

| | | |
|---|---|---|
| Carbohydrate content | 170 | 220 |
| (μg)^{a} | | |
| Nr of repeating units | 12 | 13 |

^{a}does not include the contribution of O-acetyl groups, which may be attached to the repeating Gal residues.

Monosaccharide analysis of Sm LPS (Table 13) showed a completely different composition as that for Sl LPS in accordance with its molecular structure containing Man-Rha-Gal repeating units. As described above, the molar ratio for Gal is more than the expected 1.0 in the repeating unit partly by the contribution of Gal residues in the core region.

Likewise, equimolar ratios are expected for Glc, Man, Rha and Gal as these residues form a repeating unit in St LPS (Table 14). It should be noted here that abequose is also part of the repeating unit, but is not in the analysis applied. Batch-St2003.2, however, contains less oxidizable Hep and KDO, which may affect the efficacy of the immobilization of LPS from this preparation. At the other hand, Batch St2003.2 contains much more carbohydrate than batch St2003.1, namely, 249 μg relative to 154 μg in 0.5 mg LPS, respectively.

TABLE 14
Monosaccharide analysis of S. typhimurium LPS. Batches St2003.1
and St2003.2 were fortified with Hb at 15% (m/m) and 25% (m/m),
respectively. Molar ratios were determined on the basis of two
GlcN and one GlcNAc residues (detected as three GlcNAc
residues) present in the core and lipid A regions (referred to
as CORE) and on the basis of one Man residue in the repeating
unit (referred to as UNIT). Normalized GlcNAc and Man residues
are indicated by underlining. Carbohydrate content was
determined in 0.5 mg LPS preparations.

| Molar ratio | |
|---|---|
| Batch | Batch |
| St2003.1 | St2003.2 |

| Monosaccharide | CORE | UNIT | CORE | UNIT |
|---|---|---|---|---|
| Gal | 24.7 | 1.4 | 24.2 | 1.4 |
| Glc | 17.0 | 1.0 | 16.2 | 1.0 |
| GlcNAc | 3.0 | + | 3.0 | + |
| Hep | 2.8 | + | 2.6 | + |
| KDO | 2.7 | + | 2.4 | + |
| Man | 20.5 | 1.0 | 19.2 | 1.0 |
| Rha | 19.9 | 1.1 | 19.5 | 1.2 |

| | | |
|---|---|---|
| Carbohydrate content | 154 | 249 |
| (μg)^{a} | | |
| Nr of repeating units | 15 | 14 |

^{a}does not include the contribution of (O-acetylated) Abe residues.

2.2 Protein-Supported Immobilization of LPS

Unexpectedly, intact LPS poorly coupled through its KDO-carboxylic acid function to EDC/NHS-activated carboxymethylated dextran, and no significant responses of reference sera were observed. To improve immobilization of LPS to the sensor chip, LPS was oxidized using sodium periodate to create reactive aldehyde groups in its carbohydrate constituents, which would allow the so-called aldehyde coupling procedure, i.e., condensation of aldehyde with a hydrazide function into a hydrazone linkage followed by reduction to a hydrazide product. Without oxidation, LPS had indeed little potential to immobilize to a surface of a CM5 chip coated with carbohydrazide (results not shown).

Coupling of oxidized LPS, however, gave disappointing reactivity with reference sera, probably as a result of insufficient immobilization of the antigens. Commercially acquired phenol-extracted LPS, either intact or detoxified (i.e., cleavage of lipid. A), from S. enteritidis gave, low responses when immobilized after oxidation, namely 187 RU and 167 RU, respectively. It must be noted, however, that besides poor coupling, oxidation may have destroyed a part of the antigenic structures, which may give poor serological responses.

The degree of oxidation was investigated by monosaccharide analysis of Se LPS (Table 10). This analysis revealed that relative amounts of KDO, Hep and GlcNAc, which are constituent of the core region and not of the repeating antigenic units in the PS part, were significantly reduced compared to non-oxidized Se LPS. Importantly, monosaccharide residues part of the PS, and thus antigenic structures, apparently remained intact under the mild oxidation conditions, which were applied.

TABLE 15
Biosensor responses following immobilization and responses of
reference antisera flowed over chip surfaces, which were prepared with oxidized and
non-oxidized Se or St LPS in the presence of 15% (m/m) porcine Hb.

| | | Biosensor response (RU) | | | | | |
|---|---|---|---|---|---|---|---|
| Type of | Periodate | Level | | | | | |
| LPS | treatment | of immobilization | α-O4^{a} | α-O5 | α-O9 | α-O12 | O-poly A-S |
| Se | No | 6515 | 1 | 9 | 6 | 9 | 4 |
| St | No | 4402 | 2 | 156 | 1 | 5 | 0 |
| Se | yes | 4265 | −21 | −3 | 77 | 202 | 159 |
| St | yes | 5950 | 302 | 5005 | −12 | 225 | 137 |

^{a}anti-serum against indicated O-antigen was tested

TABLE 16
Biosensor responses following immobilization and responses of
reference antisera flowed over chip surfaces, which were prepared with oxidized
St LPS (batch St2003.1) in the presence of 7% (m/m) of the indicated protein.

| | Biosensor response (RU) | | | | | | | | | | | |
|---|---|---|---|---|---|---|---|---|---|---|---|---|
| Protein | Level of | | | | | | O-poly | | | | | |
| added | immob.^{a} | O4 | O5 | O9 | O12 | O-poly E | A-S | C-SPF | C-St | C-Se | C-Si | C-Spg |
| BSA^{b} | 344 | n.d.^{c} | n.d. | n.d. | n.d. | n.d. | n.d. | n.d. | n.d. | n.d. | n.d. | n.d. |
| BSA | 7450 | 19 | 216 | 1 | 18 | 4 | 14 | 5 | 18 | 21 | 12 | 42 |
| Hb | 3410 | 319 | 3874 | 4 | 192 | 3 | 203 | 11 | 151 | 140 | 51 | 762 |
| Mb | 815 | 59 | 773 | 3 | 45 | 2 | 47 | 8 | 46 | 34 | 20 | 152 |
| Ob | 5540 | 76 | 926 | 10 | 56 | 12 | 56 | 12 | 47 | 45 | 25 | 177 |

^{a}level of immobilization;
^{b}BSA was added to oxidized and desalted LPS;
^{c}immobilization of LPS was considered too low for further reference sera analysis.

To optimize the binding of LPS and consequently improve detection of binding antibodies from sera, oxidation of LPS was then executed in the presence of a protein to allow the formation of protein-LPS complexes through Schiff-base reactions between proteinaceous amines and aldehyde functions of LPS. Indeed, commercially available TCA extracted Se LPS, containing considerable amounts of bacterial proteins, gave improved immobilization at 562 RU compared to phenol-extracted and ion-exchange chromatography-purified Se LPS at 208 RU.

Oxidation of LPS was necessary, as mixtures containing non-oxidized LPS and protein show relatively high immobilization levels but insignificant specific responses (Table 15). In addition, protein addition was only beneficial prior to oxidation of LPS, as addition of BSA to oxidized and desalted St LPS gave acceptable immobilization levels but no expected serological responses (Table 16). In a similar way, Hb yielded relatively high immobilization levels but no serological responses, as expected (results not shown).

For method improvement purposes, proteins with a relatively high degree of homology of their primary and secondary structures between homeothermic vertebrate species and occurring in serology-suitable matrices were selected for further investigations. For that reason, the performance of chicken ovalbumin, porcine hemoglobin, bovine serum albumin or porcine myoglobin fortified (7%, m/m) St LPS was compared (Table 16). Hemoglobin clearly gave the best improvement of immobilization levels together with the best expected antigen-antibody reactivity profile. In the presence of Hb, in particular O12, poly O A-S and C-St reference sera gave better responses.

This, experiment was repeated with the addition of BSA, Hb and Mb at levels indicated in Tables 17 to 20 using batches Se2005.1, Sg2005.1, Sl2005.1 and St2005.1. This time, O4 and O5 bound to immobilized St LPS as expected (Table 20). Evaluation of these results summarized in. Tables 16 to 20 revealed that when considering all expected responses simultaneously per LPS type, the addition of Hb gave highest specific responses compared to the addition of BSA and Mb. Furthermore, in most cases, standard deviations that occurred with Hb as the supportive protein were more favorable than those for the addition of BSA and Mb. Hemoglobin was, therefore, selected for further experimentation.

It was observed that the O-poly A-S antisera probably contains a low anti-serogroup C₁ and C₂ titers, as in the case of testing immobilized Sg LPS (Table 18) and Sl LPS (Table 19) where relatively low responses are found.

TABLE 17
Biosensor responses in response units (RU) following
immobilization and responses of reference antisera flowed over chip surfaces, which were
prepared with Se LPS oxidized in the presence of 15% (m/m) of the indicated protein. Values
were corrected for the C-SPF responses, which are listed as well. Standard deviations are
indicated in brackets (N = 5, except for chicken and swine sera N = 4).

| | | Antiserum tested | | | | | | | | |
|---|---|---|---|---|---|---|---|---|---|---|
| | Immob. | | | O-poly | Anti- | | | | | |
| protein | level^{a} | O9 | O12 | A-S | serogroup D | C—Se | C—St | C-Spg | P—St | C-SPF |
| Hb | 3403 | 72 (4) | 268 (1) | 160 (2) | 187 (3) | 197 (6) | 55 (7) | 1753 (5) | 76 (13) | 87 |
| Mb | 4228 | 47 (4) | 242 (3) | 133 (5) | 158 (4) | 167 (9) | 40 (12) | 1674 (6) | 80 (16) | 107 |
| BSA | 4883 | 31 (11) | 185 (2) | 99 (16) | 126 (5) | 126 (11) | 28 (30) | 1277 (4) | 68 (24) | 74 |

^{a}level of immobilization.

TABLE 18
Biosensor responses in response units (RU) following
immobilization and responses of reference antisera flowed over chip
surfaces, which were prepared with Sg LPS oxidized in the presence of
50% (m/m) of the indicated protein. Values were corrected for the C-SPF
responses, which are listed as well. Standard deviations are indicated in
brackets (N = 5, except for the chicken and swine sera N = 4).

| | | Antiserum tested | | | | | |
|---|---|---|---|---|---|---|---|
| | Immob. | | | | Anti- | | |
| protein | level^{a} | O6, 7 | O8 | O-poly A-S | serogroup C | P-Sl | C-SPF |
| Hb | 5542 | 318 (1) | 249 (11) | 69 (3) | 145 (10) | 52 (17) | −1 |
| Mb | 9880 | 237 (1) | 211 (12) | 45 (7) | 81 (26) | 162 (34) | 0 |
| BSA | 10344 | 196 (4) | 124 (8) | 19 (24) | 61 (15) | 110 (22) | −6 |

^{a}level of immobilization.

TABLE 19
Biosensor responses in response units (RU) following
immobilization and responses of reference antisera flowed over chip
surfaces, which were prepared with Sl LPS oxidized in the presence of
50% (m/m) of the indicated protein. Values were corrected for the C-SPF
responses, which are listed as well. Standard deviations are indicated in
brackets (N = 5, except for chicken and swine sera N = 4).

| | | Antiserum tested | | | | | |
|---|---|---|---|---|---|---|---|
| | Immob. | | | Anti- | | | |
| protein | level^{a} | O6, 7 | O-poly A-S | serogroup C | C—Si | P-Sl | C-SPF |
| Hb | 8180 | 125 (21) | 16 | 167 (4) | 377 (12) | 46 (7) | −17 |
| Mb | 10819 | 97 (24) | 8 | 115 (0) | 349 (3) | 96 (26) | −24 |
| BSA | 11364 | 24 (5) | −33 | 40 (5) | 200 (2) | 68 (20) | 76 |

^{a}level of immobilization

TABLE 20
Biosensor responses in response units (RU) following
immobilization and responses of reference antisera flowed over chip surfaces,
which were prepared with St LPS oxidized in the presence of 15% (m/m) of the
indicated protein. Values were corrected for the C-SPF responses, which are
listed as well. Standard deviations are indicated in brackets (N = 5, except
for chicken and swine sera N = 4).

| | | Antiserum tested | | | | | | | | |
|---|---|---|---|---|---|---|---|---|---|---|
| | Immob. | | | | O-poly | Anti- | | | | |
| protein | level^{a} | O4 | O5 | O12 | A-S | serogroup B | C—St | C-Spg | P—St | C-SPF |
| Hb | 3355 | 419 (3) | 494 (7) | 212 (2) | 107 (1) | 809 (5) | 398 (14) | 619 (24) | 272 (5) | 9 |
| Mb | 2826 | 353 (8) | 432 (4) | 176 (8) | 80 (9) | 722 (2) | 356 (20) | 532 (24) | 246 (14) | 17 |
| BSA | 5588 | 388 (7) | 304 (7) | 158 (16) | 82 (16) | 542 (4) | 324 (24) | 324 (16) | 244 (17) | 4 |

^{a}level of immobilization.

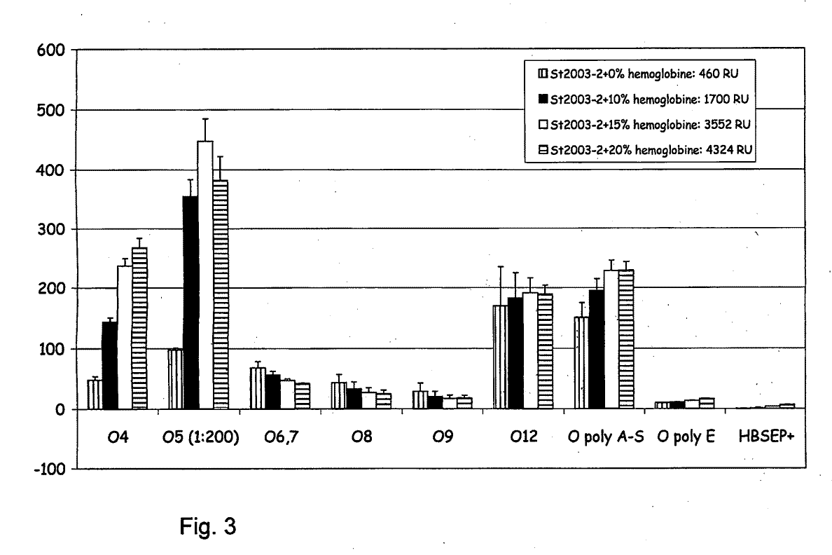

Immobilization levels and expected reactivity of agglutination sera with S. enteritidis LPS (batch Se2003.1), S. goldcoast (batch Sg2003.2), S. livingstone (batch Sl2003.1), S. typhimurium (batch St2003.1) and S. meleagridis (Batch-Sm2003.1) revealed a correlation with relative amount of Hb added before oxidation (see, for example, FIG. 3). It was also found that for a Salmonella serovar-specific LPS type, the optimum Hb concentration was also production batch dependent.

On guidance of maximum response of the expected antigenic profile using a panel of standard and reference control sera and on guidance of low responses from avian SPF reference sera, optimum Hb concentration was determined for each type and for each batch of LPS (Table 21). In the text, oxidized, hemoglobin-containing LPS preparations are further referred to as LPS^{ox}-Hb preparations.

It should be noted that, in any experiment, immobilization levels did not correlate with serological responses but correlated with the amount of Hb that was added.

TABLE 21
Effect of hemoglobin and LPS concentration on the final
immobilization level of LPS derived from S. enteritidis (Se)
and S. typhimurium (St).

| | Se LPS | | St LPS | |
|---|---|---|---|---|
| LPS concentration | 10% | 15% | 10% | 15% |
| (μg/ml) | (m/m) Hb^{a} | (m/m) Hb | (m/m) Hb | (m/m) Hb |
| 25 | | | | 9197 |
| 62.5^{b} | 2194-2065 | 3173 | | |
| 62.5^{b} | 3400-3000 | | | |
| 62.5^{b} | | | 8414 | |
| 62.5^{b} | 2374 | | | |
| 125 | 3940-3647 | | | |
| 250 | 3370 | | 10645 | |

^{a}Concentration of Hb relative to LPS
^{b}prepared on separate sensor channels.

The effect of dilution of Se LPS and St LPS before oxidation in the presence of 10% (m/m) or 15% (m/m) Hb relative to LPS, respectively, was investigated (Table 21). These results did not clearly reveal a correlation between LPS concentration and coupling level.

TABLE 22
Relationship between immobilization level of LPS^{ox}-Hb and serologic responses of
O-antigen antiserum and avian control sera. Fields for which a serological
response was expected are shaded in a green color and values within the field
are underlined. Control sera were diluted in HBS-EP containing 0.5 M sodium
chloride and 0.5% (m/v) carboxymethylated dextran.

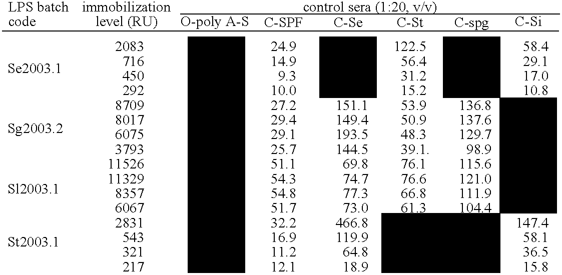

2.3 LPS Stability and Robustness of Immobilization

Reproducibility and repeatability of the immobilization of LPS and the specific response of reference sera were tested. The Se, Sg, Sl and St LPS preparations were oxidized in the presence of their corresponding optimal Hb concentration, desalted, and then stored in solution at 4° C. in the dark. Under identical conditions, but accounting with the variability generally observed for immobilization of molecules at a biosensor surface, immobilization levels of oxidized LPS were either comparable in the cases of the Sl (5% RSD) and St (8% RSD) batches or tended to increase in the cases of the Se (13% RSD) and Sg (8% RSD) batches over two months of storage (Table 23 through. Table 26). The responses of reference sera were probed on the prepared biosensor chips as well. Inspection of these did not reveal a correlation between immobilization level and specific response. For example, immobilization levels of Se LPS and Sg LPS may tend to increase over time; this increase was not reflected in the response of the binding of serum antibodies to the immobilized antigens. The relative standard deviation varies between 12% and 38%.

When considering the first three measuring days (day 0, day 7 or 10, and day 14 or 17) the variance is greatly reduced from 3.5% to 23% (results not shown). Tables 27 to 30 summarize the repeatability of the method at several moments over a 12-months period. At each analysis time point, a fresh aliquot of Hb-fortified LPS was oxidized, immobilized and analyzed and thus reflects the sum of variability of several steps.

TABLE 23
Response of agglutination and reference antisera with LPS^{ox}-Hb
Se2003.1 prepared at day 0 and stored at 5° C.. to 8° C. The LPS
preparation was immobilized and tested after oxidation at the days
indicated.

| | | Se2003.1 oxidized in presence of 15% Hb | | | | |
|---|---|---|---|---|---|---|
| Day of | immobilization | | | O- | | C-Spg |
| analysis | level (RU) | O9 | O12 | poly A-S | C—Se | (1:100) |
| 0 | 2708 | 446 | 302 | 330 | 2597 | 3535 |
| 10 | 3007 | 352 | 258 | 252 | 2205 | 3726 |
| 17 | 3194 | 472 | 320 | 292 | 2520 | 3586 |
| 31 | 3642 | 509 | 356 | 340 | 1486 | 3727 |
| 62 | 3640 | 361 | 223 | 156 | 1676 | 2329 |
| Average | 3238 | 428 | 292 | 274 | 2097 | 3381 |
| St. dev. | 407 | 69 | 52 | 74 | 498 | 594 |
| RSD (%) | 13 | 16 | 18 | 27 | 24 | 18 |

TABLE 24
Response of agglutination and reference antisera with LPS^{ox}-Hb
Sg2003.2 prepared at day 0 and stored at 5° C. to 8° C..
The LPS preparation was immobilized and tested after oxidation
at the days indicated.

| | | Sg2003.2 oxidized in | | |
|---|---|---|---|---|
| Day of | immobilization | presence of 50% Hb | | |
| analysis | level (RU) | O6, 7 | OS | O-poly A-S |
| 0 | 7262 | 668 | 465 | 117 |
| 7 | 9972 | 714 | 531 | 110 |
| 14 | 10597 | 657 | 566 | 111 |
| 20 | 11453 | 857 | 502 | 100 |
| 28 | 11866 | 1085 | 504 | 130 |
| 59 | 12002 | 484 | 364 | 58 |
| Average | 10525 | 744 | 489 | 104 |
| St. dev. | 869 | 226 | 77 | 27 |
| RSD (%) | 8 | 30 | 16 | 26 |

TABLE 25
Response of agglutination and reference antisera with LPS^{ox}-Hb
Sl2003.1 prepared at day 0 and stored at 5° C. to 8° C..
The LPS preparation was immobilized and tested after oxidation
at the days indicated.

| | | |
|---|---|---|
| | | Sl2003.1 oxidized in |
| Day of | immobilization | presence of 50% Hb |

| analysis | level (RU) | O6, 7 | O-poly A-S |
|---|---|---|---|
| 0 | 11509 | 318 | 84 |
| 7 | 13581 | 289 | 63 |
| 14 | 14870 | 250 | 54 |
| 20 | 13582 | 475 | 65 |
| 28 | 14976 | 468 | 75 |
| 59 | 14744 | 202 | 31 |
| Average | 13877 | 334 | 62 |
| St. dev. | 707 | 127 | 17 |
| RSD (%) | 5 | 38 | 27 |

TABLE 26
Response of agglutination and reference antisera with LPS^{ox}-Hb
St2003.1 prepared at day 0 and stored at 5° C.. to 8° C. The
LPS preparation was immobilized and tested after oxidation at the
days indicated.

| | | St2003.1 oxidized in presence of 15% Hb | | | | | | |
|---|---|---|---|---|---|---|---|---|
| Day of | immobilization | | | | O-poly | | | C-Spg |
| analysis | level (RU) | O4 | O5 (1:200) | O12 | A-S | C—St | C—Se | (1:100) |
| 0 | 5391 | 594 | 540 | 278 | 362 | 441 | 544 | 1634 |
| 10 | 4432 | 463 | 567 | 242 | 273 | 448 | 356 | 1442 |
| 17 | 4473 | 517 | 578 | 250 | 262 | 362 | 396 | 1294 |
| 31 | 4769 | 559 | 582 | 296 | 326 | 440 | 255 | 1464 |
| 62 | 4996 | 442 | 321 | 209 | 196 | 340 | 297 | 983 |
| Average | 4812 | 515 | 518 | 255 | 284 | 406 | 370 | 1363 |
| St. dev. | 397 | 64 | 111 | 34 | 64 | 51 | 112 | 244 |
| RSD (%) | 8 | 12 | 22 | 13 | 22 | 13 | 30 | 18 |

TABLE 27
Responses of freshly oxidized Se LPS (batch Se2003.1) on indicated time
points (in months). The LPS was isolated from bacterial cells, fortified
with 15% (m/m) Hb, dried and stored at 4° C. to 7° C. until day
of oxidation, immobilization and analysis.

| Anal- | immobi- | Se2003.1 oxidized in presence of 15% Hb | | | | |
|---|---|---|---|---|---|---|
| ysis | lization | | | O-poly | C—Se | C-Spg |
| (month) | level (RU) | O9 | O12 | A-S | (1:200, v/v) | (1:100, v/v) |
| 0 | 2708 | 446 | 302 | 330 | 2597^{a} | 3535^{b} |
| 1^{c} | 1326 | 252 | 165 | 172 | 464 | 1274 |
| 2 | 2380 | 372 | 262 | 230 | 502 | 1478 |
| 3 | 2003 | 398 | 208 | 230 | 676 | 1864 |
| 5 | 2309 | 144 | 207 | 337 | 534 | 1539 |
| 7 | 3487 | 240 | 444 | 547 | 786 | 2368 |
| 9 | 3721 | 215 | 407 | 576 | 705 | 2415 |
| 12 | 1450 | 78 | 145 | 219 | 159 | 1272 |

^{a}serum was diluted 1:50 (v/v)
^{b}serum was diluted 1:100 (v/v)
^{c}LPS was diluted at another volume ratio

TABLE 28
Responses of freshly oxidized Sg LPS (batch Sg2003.2) on indicated
time points (in months). The LPS was isolated from bacterial cells,
fortified with 50% (m/m) Hb, dried and stored at 4° C. to 7° C.
until day of oxidation, immobilization and analysis.

| | | Sg2003.2 oxidized in | | |
|---|---|---|---|---|
| Analysis | immobilization | presence of 50% Hb | | |
| (month) | level (RU) | O6, 7 | O8 | O-poly A-S |
| 0 | 7262 | 668 | 465 | 117 |
| 1 | 7428 | 929 | 451 | 100 |
| 2 | 9023 | 639 | 459 | 91 |
| 3 | 13152 | 474 | 606 | 119 |
| 5 | 8087 | 549 | 446 | 294 |
| 7 | 9724 | 622 | 382 | 286 |
| 9 | 8870 | 692 | 508 | 364 |
| 12 | 7088 | 491 | —* | 281 |

TABLE 29
Responses of freshly oxidized S1 LPS (batch Sl2003.1) on indicated
time points (in months). The LPS was isolated from bacterial cells,
fortified with 50% (m/m) Hb, dried and stored at 4° C. to 7° C.
until day of oxidation, immobilization and analysis.

| | | |
|---|---|---|
| Analysis | immobilization | Sl2003.1 |

| (month) | level (RU) | O6, 7 | O-poly A-S |
|---|---|---|---|
| 0 | 11509 | 318 | 84 |
| 1 | 11442 | 417 | 62 |
| 2 | 12280 | 287 | 55 |
| 3 | 13152 | 231 | 65 |
| 5 | 11896 | 217 | 159 |
| 7 | 11563 | 271 | 149 |
| 9 | 10882 | 298 | 191 |
| 12 | 10138 | 204 | 131 |

TABLE 30
Responses of freshly oxidized St LPS (batch St2003.1) on indicated
time points (in months). The LPS was isolated from bacterial cells, fortified
with 15% (m/m) Hb, dried and stored at 4° C. to 7° C. until day of oxidation,
immobilization and analysis.

| | | St2003.1 oxidized in presence of 15% Hb | | | | | |
|---|---|---|---|---|---|---|---|
| Analysis | immobilization | | O5 (1:200, | | O-poly | | C-Spg |
| (month) | level (RU) | O4 | v/v) | O12 | A-S | C—St | (1:200, v/v) |
| 0 | 5391 | 594 | 540* | 279 | 362 | 441 | 1634^{a} |
| 1^{b} | 3487* | 339 | 470 | 181 | 207 | 352 | 597 |
| 2 | 4623 | 482 | 325 | 257 | 242 | 411 | 612 |
| 3 | 4079 | 425 | 608 | 187 | 235 | 982 | 624 |
| 5 | 6873 | 359 | 369 | 167 | 302 | 393 | 605 |
| 7 | 5410 | 681 | 734 | 334 | 453 | 559 | 837 |
| 9 | 4008 | 638 | 664 | 278 | 441 | 524 | 824 |

^{a}serum was diluted 1:100 (v/v);
^{b}Following oxidation, LPS-containing solution was diluted twice instead of once.

### Example 2

### SPR Biosensor for Detection of Egg Yolk Antibodies Reflecting Salmonella Enteritidis Infections_{[AAB4]}

### 1. Introduction

Salmonella is one of the major causes of bacterial gastro-enteritis of humans (Fischer, 2004; van Duynhoven et al., 2005). In the Netherlands, between 1994-1998, Salmonella enterica serovar enteritidis (S.e.) was the most often isolated serovar (Pelt et al., 1999). Within this serovar, eggs and egg products were the most important source of infection. Despite several control measures, approximately 9% of the Dutch layer flocks become infected annually. As egg contamination with Salmonella continues to be a threat for public health, it is important to detect an infection of a flock as soon as possible by, an adequate surveillance program.

The current Dutch monitoring system in layer finisher hens is based on serology (Bokkers, 2002). The aim is to reduce the prevalence of S.e. and S. typhimurium in the layer sector. Sampling, however, occurs only twice: before and at the end of the laying period. The current surveillance program, therefore, cannot detect all infections of flocks during the laying period, and farmers cannot “guarantee” that their products are from Salmonella-free layers.

Consequently, the surveillance program should be improved. As an alternative to current serology, testing of eggs for antibodies could be performed. Egg sampling has the advantage that it can be performed on egg packing plants, in a high sampling frequency and with large sample sizes.

Tests for detection of antibody in eggs have been developed and used before. The existing tests are often based on enzyme-linked immunosorbent assays (ELISA) using different (combinations of) antigenic components of Salmonella spp. (see, for examples, Refs. Gast et al., 2002 and 1997; Skov et al., 2002; Holt et al., 2000; Desmidt et al., 1996; Sachsenweger et al., 1994; Van Zijderveld et al., 1992). Recently, the possible suitability of biosensors for the detection of humoral response has been recognized (Bergwerff and van Knapen, 2006, accepted for publication; Bergwerff and van Knapen, 2003; Jongerius-Gortemaker, 2002; Pyrohova et al., 2002; Vetcha et al., 2002; Li et al., 2002; Liu et al., 2001; Uttenthaler et al., 1998). A biosensor consists of a re-usable immobilized biological ligand that “senses” the analyte, and a physical transducer, which translates this phenomenon into an electronic signal (Jongerius-Gortemaker et al., 2002). The use of biosensors promises the possibility of high throughput analyses, and also the detection of multiple serovars or serogroups within a family of infectious disease agents, or antibodies against these agents, in a single run. This offers the opportunity to improve surveillance programs, as more samples can be tested in a higher frequency during the laying period.

This example evaluates the sensitivity, specificity and discriminatory capacity of a surface plasmon resonance (SPR) biosensor (Biacore 3000) antibody detection test in egg yolk based on the lipopolysaccharide (LPS) of Salmonella enterica serovar enteritidis and compares the results to those obtained with a g,m flagellin-based commercial ELISA test kit and a LPS-based commercial ELISA test kit for detection of egg antibodies by creating and analyzing receiver operating characteristic (ROC) curves.

### 2. Materials and Methods

2.1 SPR Biosensor Method

We adapted the surface plasmon resonance (Biacore 3000 by Biacore AB, Uppsala, Sweden) detection method of serum antibodies against Salmonella enteritidis using LPS-antigen as developed by Bergwerff et al. (in prep) to egg yolk. The first adjustment to this method was made in the sample preparation. After separation of egg yolk and egg white, the egg yolk was diluted 1:5 (v/v) in 10 mM HEPES buffer at pH 7.4, containing 3 mM EDTA, 0.15 M sodium hydrochloride, 0.005% (v/v) surfactant P20 (Biacore AB, Sweden), and additional 0.85 M sodium chloride (Merck, Darmstadt, Germany), 1% (m/v) carboxymethylated dextran (Fluka Chemie, Buchs, Germany) and 0.05% (v/v) TWEEN®80 (Merck, Germany). It was mixed with glass pearls, centrifuged at 15,000 g at ambient temperature for 25 minutes; the supernatant was filtrated over a 0.45-μm filter (Schleicher & Schnell, Dassel, Germany).

A second adaptation was the cleaning of the sensor chip. Following analysis of each series of 15 egg yolk samples, a solvent containing 0.5% (w/v) sodium dodecyl sulphate (Biacore AB, Sweden) was injected to remove deposited egg yolk components.

2.2 Reference Sera and Egg Yolks

Sera were used as reference in the various tests due to unavailability of sample stock of well-defined reference egg yolks. Lyophilized, defined SPF and reference sera originating from chickens infected with a) S. enteritidis, b) S. typhimurium, c) S. infantis, or d) S. pullorum were obtained from the Animal Health Service Ltd. (Deventer, Netherlands). These sera were prepared from pooled sera. Before use, lyophilized sera were reconstituted in 1 ml MILLI-Q®. Additionally, monoclonal mouse anti-Salmonella antibody anti-group B, -group C, -group D and -group E was used (Sifin, Berlin, Germany).

Internal-control egg yolks were used to establish analytical sensitivity and repeatability of the biosensor assay. They consisted of a specific pathogen-free (SPF) egg yolk sample (Animal Health Service Ltd., Netherlands) and a highly immuno-responsive pre-ovulatory follicle sample originating from experiment 2 (cf. section 2.4. below).

2.3 ELISA

Samples were assayed using sandwich enzyme immunoassay techniques. Two commercially available S.e. antibody detection kits were used; Flockscreen S.e. Guildhay (Guildford, England) and FlockChek S.e. IDEXX (Westbrook, Me., USA). The samples were analyzed according to the company's procedures.

The Guildhay S.e. indirect ELISA is based on LPS as antigen. The wells of microtiter plates were coated with LPS, 1:500 dilutions of samples were added in mono. Test results were expressed as an S/P ratio according to the following formula:

$$S / P = \frac{\begin{matrix}( optical density sample - \\ optical density negative controls )\end{matrix}}{\begin{matrix}( optical density positive controls - \\ optical density negative controls )\end{matrix}} . \qquad ( 1 )$$

The S/P ratio was interpreted using the following criteria: Egg yolk: S/P≦0.08=immuno-negative; 0.08<S/P<0.25=immuno-suspect; S/P≧0.25=immuno-positive.

The IDEXX S.e. competitive ELISA is based on g,m flagellar antigen. The wells of microtiter plates were coated with g,m flagellar antigen, 1:2 dilutions of samples were added in mono. The results were expressed as S/N ratio as follows:

$$S / N = \frac{optical density sample}{optical density negative controls} \qquad ( 2 )$$

The S/N ratio was interpreted using the following criteria: Egg yolk: S/N≧0.75=immuno-negative; 0.75<S/N<0.59=immune-suspect; S/N≦0.59=immuno-positive.

2.4 Experiments

The egg samples used in this study originated from two infection experiments.

Experiment 1. Fifteen one-week-old layer hens (Isa Brown) were housed in negative pressure high-efficiency particulate air filter (HEPA) isolators with a volume of 1.3 m³ and fitted with a wire floor of 1.1 m², and applying a 12-hour light to 12-hour dark photoperiod rhythm. The isolators were ventilated at a rate of approximately 30 m³¹ hour. During the growing period, no Salmonella could be cultured from bedding. The chickens were provided with non-medicated feed and water ad libitum. They were housed, handled and treated following approval by the institutional animal experimental committee of the Dutch Animal Health Service Ltd. in accordance with the Dutch regulations on experimental animals. All hens were inoculated orally once with 1×10⁸ CFU per bird in week 20 of the experiment using S. enteritidis CL344 (Animal Health Service Ltd., Deventer; The Netherlands). Before and after inoculation, eggs were collected on a daily basis, but not labeled individually and not dated. The eggs were stored at ambient temperature for four weeks and subsequently at 4° C. The experiment ended in week 22 and produced 147 “positive” and 71 “negative” samples.

Experiment 2. This experiment is described in detail by Van Eerden et al. (2005, in prep). In short, 128 15-week-old layer hens (Lohmann Brown, 16 birds, eight replications) were divided into two groups (eight hens each) and housed individually in two climate cells, used as isolators, in the same room, under a 9-hour light and 15-hour dark photoperiod rhythm. During the growing period until inoculation, no Salmonella was cultured from feces. They were provided with non-medicated feed and water ad libitum. The animal experiment was conducted according to the Guidelines for Animal Experimentation of Wageningen University and approved by the Ethical Committee under Reference Number of 2603219. Each of sixty-four hens (16 birds, four replications) was inoculated orally once with 1×10⁸ CFU nalidixic acid-resistant S.e. (ASG, Lelystad, the Netherlands) one week after the experiment started. The other 64 birds were considered uninfected controls. Eggs were collected at day 21 and day 28 post-inoculation. Twelve times, the eggs from one climate cell were collected and pooled after cracking of the shell (cf. section 2.5 below). Four of the pooled egg samples were taken from “uninfected” climate cells. Besides the collection of pooled egg samples, ten egg samples were taken from ten individual birds, of which seven were uninfected. The egg yolk and white were mixed and stored at −20° C. The experiment ended four weeks after inoculation. Pre-ovulatory follicles were then harvested from eight uninfected and five infected individual birds.

2.5 Preparation of Egg Samples

Eggs from Experiment 1 were prepared in the following manner. To facilitate aseptic preparation, the eggshells were disinfected with a 70% (v/v) aqueous ethanol. Subsequently, the eggs were cracked and the contents were collected in sterile petri dishes. A volume of 1 ml egg yolk was collected using a sterile disposable syringe and portioned in 200 μl fractions. Each fraction was diluted with a buffer appropriate for either SPR biosensor or ELISA analysis, and then stored at −20° C.

Likewise, pooled egg yolk and white and pre-ovulatory follicles obtained from Experiment 2 were fractionated, diluted and stored at −20° C.

2.6 Evaluation of the SPR Biosensor Method

2.6.1 Analytical Sensitivity and Specificity

To establish the limit of detection of the assay, eight 1:2 (v/v) serial dilutions of a highly immuno-responsive egg yolk sample and a SPF-negative control sample were analyzed in triplicate by the SPR biosensor. Analysis of variance (ANOVA) was performed using SPSS (SPSS for Windows, Standard Version, 1999) to evaluate differences in SPR biosensor responses obtained after injection of the serially diluted control samples.

Reference sera were used to spike a SPF yolk to test the specificity of the SPR biosensor assay. For this purpose, egg yolk was spiked with 1) S. enteritidis—(serogroups D), 2) S. infantis—(serogroups C), 3) S. pullorum—(serogroups D), and 4) S. typhimurium—(serogroups B) reacting antisera. These samples were diluted by their volumes, either at a rate of 1:100 (1, 2 and 3) or at 1:50 (4). Further specificity testing was performed by spiking SPF egg yolk with 1:100 (v/v) diluted mouse monoclonal antibody reacting with Salmonella serogroups B, C, D and E.

2.6.2 Repeatability

The repeatability of the SPR biosensor assay was assessed by running the highly immuno-responsive egg yolk sample and the SPF-negative control egg yolk sample twice on a single day and on three consecutive days (in triplo). Means, standard deviations (SD) and percent coefficient of variation (% CV) values were calculated in Excel 2000 (Microsoft software package).

2.6.3 ROC Curves

Receiver operator characteristic (ROC) curves were generated using the results from the SPR biosensor and ELISA analyses to assess the test performances of each assay (Zweig and Campbell, 1993). Using SPSS, the overall accuracy of each assay was calculated from the integrated area under the curve (AUC), corresponding standard error (SE) and the probability of the null hypothesis of the true AUC being 0.5. By use of non-parametric ROC analysis (Metz et al., 1998), the accuracy of SPR biosensor assay detection of antibodies against S.e. was compared with the accuracy of the two ELISAs. The gold standard was the infection status of the experimental group.

2.6.4 Diagnostic Sensitivity and Specificity

In a ROC curve, the true positive rate (sensitivity) is plotted in function of the false positive rate (100-specificity) for different cut-off points of a parameter. Each point on the ROC curve represents a sensitivity/specificity pair corresponding to a particular decision threshold. Thus, the maximum diagnostic sensitivity at the highest diagnostic specificity for the SPR biosensor assay and the two ELISAs were calculated using SPSS. For the SPR biosensor test using the samples from Experiment, the maximum diagnostic specificity at the highest diagnostic sensitivity and the optimal combined diagnostic sensitivity and specificity were also calculated.

### 3. Results

3.1 Analytical Sensitivity and Specificity

A 1:640 (v/v) dilution of the highly immuno-responsive egg yolk sample was, at 50 RU, the highest dilution tested that differed significantly (P<0.001) from the negative control.

The test signal of the SPF egg yolks spiked with S. enteritidis—(1:100, 145 RU), S. pullorum—(1:100, 1012 RU) or S. typhimurium—(1:50, 58 RU) positive sera were above the optimized cut-off value of 52 RU (cf. section 3.4.1 below) and considered positive, as was the SPF egg yolk spiked with mouse anti-Salmonella group D (1:100, 130 RU). Non-spiked SPF yolk was found to be negative, i.e., average response was 30 RU. The yolks spiked with S. infantis—(1:100, 24 RU) positive serum and mouse antiserum against Salmonella serogroups B (1:100, 27 RU), C (1:100, 16 RU), and E (1:100, 15 RU) were also below the cut-off value.

3.2 Repeatability

The coefficient of variation within a single day was 1% for the highly immuno-responsive egg yolk sample and 13% for the negative sample. The coefficient of variation from day-to-day during three days was 2% for the positive sample and 17% for the negative sample.

3.3 Threshold Determination

3.3.1 ROC Analysis

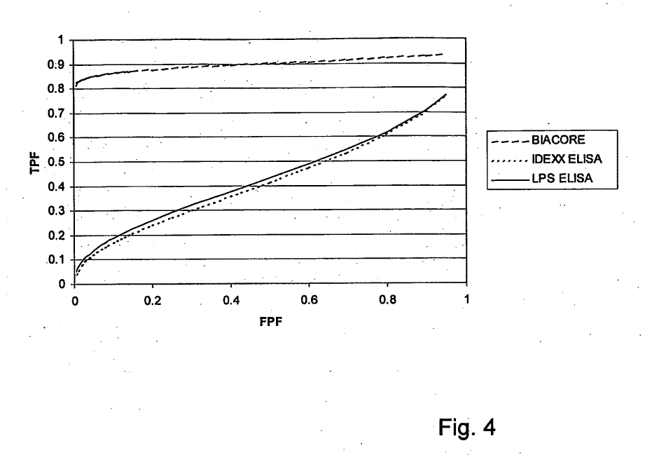

ROC analysis was performed on the assay results of 71 and 135 egg yolk samples from uninfected and infected chickens, respectively, from Experiment 1 (not all tests were performed on 12 samples from infected chickens). Integrated areas under ROC curves were 0.892 (SE 0.024, P<0.001) for the SPR biosensor assay; 0.432 (SE 0.039, P=0.103) for the IDEXX ELISA and 0.430 (SE 0.039, P=0.096) for the Guildhay ELISA (Table 31). The ROC curves are depicted in FIG. 4. The integrated area (AUC), and thus the overall accuracy, for the SPR biosensor assay was significantly larger than those of the IDEXX (Z=11.5, P<0.001) and Guildhay ELISA (Z=10.5, P<0.001).

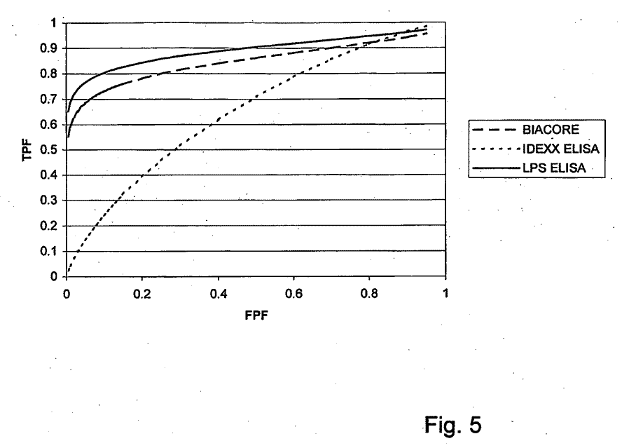

ROC analysis was also performed for four combined egg white and yolk samples and 15 egg yolk samples from uninfected chickens, and eight combined egg white and yolk samples and eight egg yolk samples from infected chickens from Experiment 2. The integrated areas under ROC curves were 0.811 (SE 0.082, P=0.002) for the SPR biosensor assay, 0.615 (SE 0.098, P=0.098) for the IDEXX ELISA and 0.870 (SE 0.064, P<0.001) for the Guildhay ELISA (Table 32 and FIG. 5). The AUC of the —SPR biosensor assay was significantly larger (Z=1.9, P=0.055) than that of the IDEXX ELISA, but not different from that of the Guildhay ELISA (Z=−1.0, P=0.322).

3.4 Performance Estimates

3.4.1 Diagnostic Sensitivity and Specificity Estimates

With respect to the results of the samples acquired from Experiment 1, samples from the uninfected population gave biosensor responses ranging from 6 to 50 RU. The responses of the samples from the infected population-ranged from 11 to 3584 RU. At a cut-off value of 52 RU, 24 out of 135 samples had to be considered immuno-negative.

A cut-off value of 52 RU yielded the highest possible diagnostic specificity estimate of 100% (with a 95% exact confidence interval (CI) of 95% to 100%) and a diagnostic sensitivity estimate of 82% (95% CI: 76% to 98%) for the SPR biosensor assay test. A cut-off value of 10 RU yielded the highest possible diagnostic sensitivity estimate of 100% (95% exact CI: 97% to 100%) and a specificity estimate of 1% (95% CI: 0% to 4%). A cut-off value of 42 RU yielded the optimal combined diagnostic sensitivity and specificity: 84% (95% CI: 77% to 90%) and 99% (95% CI: 96% to 100%), respectively.

At a cut-off value of OD₅₅₀ₙₘ 0.11, the IDEXX ELISA had a diagnostic specificity of 100% and a sensitivity of 1% (95% CI: 0% to 3%). The OD₅₅₀ₙₘ of the samples from the uninfected population ranged from 0.174 to 1.377, i.e., in excess of the cut-off value at 0.11. Of the positive population, 145 out of 147 samples had to considered immuno-negative at the chosen cut-off value, namely, corresponding OD₅₅₀ₙₘ ranged from 0.042 to 1.572. The Guildhay ELISA had a diagnostic specificity of 100% and a sensitivity of 16% (95% CI: 10% to 22%) at a cut-off value of OD₆₅₀ₙₘ 0.12. None of the samples from the uninfected population showed OD₆₅₀ₙₘ values in excess of 0.12 (0.051 to 0.093). In case of the positive population, 124 out of 147 samples had to be considered immuno-negative. The OD₆₅₀ₙₘ of these samples ranged from 0.048 to 1.471.

In the case of Experiment 2, a cut-off value of 542 RU yielded the highest possible diagnostic specificity estimate of 100% (95% exact CI: 82% to 100%) and a diagnostic sensitivity estimate of 63% (95% CI: 39% to 86%) for the SPR biosensor assay test. The samples from the uninfected population had RU values ranging from 101 to 448. The infected population values ranged from 117 to 3012 and 6 out of 16 samples had negative test results. At a cut-off value of OD₅₅₀ₙₘ 0.49, the IDEXX ELISA had a diagnostic specificity of 100% and a sensitivity of 19% (95% CI: 0% to 38%). None of the samples from the uninfected population had OD₅₅₀ₙₘ values of less than 0.49. The values ranged from 0.537 to 1.621. Of the positive population, 13 out of 16 samples had negative test results at the chosen cut-off value. The values ranged from 0.134 to 1.630. The Guildhay ELISA had a diagnostic specificity of 100% and a sensitivity of 67% (95% CI: 43% to 91%) at a cut-off value of OD₆₅₀ₙₘ 0.14. None of the samples from the uninfected population had OD₆₅₀ₙₘ values of more than 0.14. The values ranged from 0.072 to 0.140. Of the positive population, five out of 15 samples had negative test results (one sample could not be tested). The values ranged from 0.086 to 2.144.

### 4. Discussion

The aim of this study was to quantify the test characteristics of the SPR biosensor for the detection of S. e. antibodies in eggs. The results showed that the SPR biosensor assay performed significantly better than the two commercially available ELISAs for samples from Experiment 1. The combined optimal diagnostic sensitivity and specificity of the SPR biosensor was 84% (77% to 90%) and 99% (96% to 100%), respectively. Neither the g,m flagellin-based IDEXX ELISA, nor the LPS-based Guildhay ELISA were able to detect S.e. infection with a higher combined diagnostic sensitivity and specificity using this test panel. This study indicates that an SPR biosensor assay could be a new and powerful tool for monitoring. Salmonella enterica serovar enteritidis infections in layer flocks through antibody detection in eggs.

The SPR biosensor assay offers the possibility of detecting infections in a fast and reliable way. The high quality of the test and the technical and animal welfare advantages of egg collection are good reasons to explore its use for screening of populations. In addition, the configuration of the applied SPR biosensor from Biacore allows the simultaneous detection of antibodies to multiple Salmonella serovars in a single run in a single sensor channel or in separate sensor channels on the same sensor chip (results not shown). This could be of significance because it is well known that serovars differ over countries- and over time (see, for example, refs. Guerin et al., 2005; van Duijnkeren et al., 2002).

The test evaluation was carried out using eggs from two experiments that were not carried out specifically for this test evaluation, possibly influencing test performance. The “positive” eggs were probably collected at a time point that humoral response was developing in the exposed chickens. These “premature” eggs were analyzed and their false-negative results interfere with the evaluation of the assays. Could it have been possible to exclude eggs until two weeks post-infection, the diagnostic sensitivity of each test, ELISA or SPR biosensor, would have been improved.

Antibody detection in serum is more sensitive than in eggs because the appearance of antibodies in eggs is preceded by the appearance in serum by a week (Gast and Beard, 1991; Sunwoo et al., 1996; Skov et al., 2002). However, flock sensitivity of tests for antibodies in eggs can be improved by taking more samples, which is easier when using eggs.

The biosensor performance (AUC 0.892) was compared to that of two commercial ELISAs (IDEXX AUC 0.432, Guildhay AUC 0.430). To our knowledge, the IDEXX ELISA was not validated for eggs, but quantitative data exist about the test's performance in comparison to other tests: Van Zijderveld et al. (1992) evaluated four different ELISAs for diagnosis of S.e. infections in experimentally infected chickens. They reported a specificity of 100% and a sensitivity of 95% for 127 egg yolks from eggs laid between 13 and 40 days after infection with S.e. In our evaluation, the IDEXX test did not perform as well as in the 1992 evaluation. An explanation could be the different sample selection, as our samples originated from infection experiments that stopped at two and four weeks after inoculation, having had less time to develop a humoral response.

Shared O-antigens, among members of Salmonella serogroups B and D are known to limit the specificity of detecting S.e. using lipopolysaccharide antigens (de Vries et al., 1998; Baay and Huis in't Veld, 1993; Hassan et al., 1990). This is confirmed by our results: the assay could not differentiate between infections with serovars enteritidis, gallinarum and typhimurium, sharing O9 and O12. As the zoonotic serovars of the three (S. typhimurium plus S. enteritidis) represent 80% of isolates identified by the national reference laboratories participating in the Enter-net surveillance network between 1998 and 2003 (Fischer et al., 2004), this finding has limited clinical relevance for the human population. The assay did differentiate between SPF egg yolk spiked with mouse anti-Salmonella group B (1:100, 27 RU) and D (1:100, 130 RU). This is not surprising because the LPS of Salmonella enteritidis has O1, O9 and O12 as somatic antigens, while the group-specific test reagents contain the following monoclonal antibodies; anti-Salmonella group B: Anti-O4, O5, O27; anti-Salmonella group D: Anti-O9.

The cut-off value from Experiment 2 was much higher than the cut-off from Experiment 1, possibly because part of our samples consisted of egg white and yolk instead of egg yolk only.

For different applications, different cut-off values may be optimal. Relative costs or undesirability of errors (false positive/false negative classifications) and the expected relative proportions of infected and uninfected hens are important parameters in the determination of the cut-off value, which affects the diagnostic value of the assay. We would suggest a cut-off value that minimizes the number of false positive results, reasoning that frequent, sampling and testing would be necessary if the assay was to be used in a surveillance program in the layer population, given the relatively low prevalence of S.e.

The SPR biosensor technique has successfully detected egg antibodies to determine experimental infections in chickens. In future screening programs, the SPR biosensor could possibly detect different analytes at the same time.

TABLE 31
ROC analysis of the results of samples derived from Experiment 1
analyzed by SPR biosensor, IDEXX and Guildhay ELISAs.

| | SPR | | Guildhay |
|---|---|---|---|
| Characteristic | biosensor assay | IDEXX ELISA | ELISA |
| Optimized cut-off | 52 RU | OD_{550 nm} 0.11 | OD_{650 nm} 0.12 |
| Diagnostic | 82 | 1 | 16 |
| sensitivity (%) | | | |
| 95% CI (%) | 76-89 | 0-3 | 10-22 |
| Diagnostic | 100 | 100 | 100 |
| specificity (%) | | | |
| 95% CI^{a} | 95-100 | 95-100 | 95-100 |
| AUC | 0.892 | 0.432 | 0.430 |
| 95% CI | 0.844-0.939 | 0.356-0.508 | 0.355-0.506 |

^{a}Fisher's exact test

TABLE 32
ROC analysis of the results of samples derived from Experiment 2
analyzed by SPR biosensor, IDEXX and Guildhay ELISAs.

| | SPR | | |
|---|---|---|---|
| | biosensor | | Gulidhay |
| Characteristic | assay | IDEXX ELISA | ELISA |
| Optimized cutoff | 542 RU | OD_{550 nm} 0.49 | OD_{650 nm} 0.14 |
| Diagnostic | 63 | 19 | 67 |
| sensitivity (%) | | | |
| 95% CI | 39-86 | 0-38 | 43-91 |
| Diagnostic | 100 | 100 | 100 |
| specificity (%) | | | |
| 95% CI^{a} | 82-100 | 82-100 | 82-100 |
| AUC | 0.811 | 0.615 | 0.870 |
| 95% CI | 0.649-0.972 | 0.424-0.806 | 0.745-0.996 |

^{a}Fisher's exact test

### Example 3

### Direct Detection of Campylobacter spp. Through Monitoring Bacteriophage Infections Using LPS-Coated Beads

### 1. Introduction

Campylobacter is the most commonly found food-borne pathogen in developed countries, causing gastroenteritis characterized by watery and/or bloody diarrhea. Campylobacter is associated with Guillain-Barré (GBS), Reiter's and hemolytic uremic (HUS) syndromes and reactive arthritis (FSAI, 2002; Lake et al., 2003; Tauxe, 2000). In the last 20 years, the infection rate of Campylobacter is still increasing in many developed countries, maybe due to the improvements in detection and reporting. In the United States of America, 2,400,000 cases of campylobacteriosis are reported annually corresponding to approximately 1% of the USA population (Tauxe, 2000).

Wild birds and domestic animals are reservoirs for Campylobacter and shed bacteria to the environment. Poultry is an important vehicle for Campylobacter infection in humans. Indeed, strains that were isolated from chickens could be isolated from patients as well (Coker, 2000).

Epidemiological studies have shown that consumption and handling of poultry meat should be considered as a major risk for human infection with C. jejuni or C. coli (FSAI, 2002). The most consistent risk factor in the United States, New Zealand and Europe has been consumption or contact with raw or undercooked poultry, accounting for 10% to 50% of all cases of campylobacteriosis (Tauxe, 2000). C. jejuni, C. coli and C. lari represent about 90% of human campylobacteriosis (Stern and Line, 2000). The infective dose of Campylobacter is considered to be low, ranging from 500-10,000 cells (FSAI, 2002).

Campylobacter are gram-negative, curve-shaped, S-shaped, or spiral-shaped bacilli having one or two flagella at one of the poles and highly motile (Christensen et al., 2001). Campylobacter grows between 30.5° C. and 45° C. at an optimum temperature of 42° C. Optimum growth is established at 10% carbon dioxide, 5% to 6% oxygen, and 85% nitrogen (FSAI, 2002).

Traditional phenotyping methods for determination of Campylobacter take four to five days and involve pre-enrichment followed by isolation from selective agar and confirmation by biochemical test. Due to the perishable nature of food items and the speed required for analysis of food products more rapidly, sensitive and specific methods are needed for cost-effective Campylobacter detection.

Immunomagnetic separation (IMS) procedures were used by Waller and Ogata (2000), Che et al. (2001), Yu et al. (2001), to concentrate C. jejuni from poultry meat without the pre-enrichment cell culture step. This approach could retrieve 10⁴ colony forming units (cfu)/g in poultry meats as detected with atomic force and fluorescence microscopy (Yu et al., 2001). IMS can potentially reduce pre-enrichment time of Campylobacter and may overcome the problems of inhibitors from food sources such as PCR inhibitors (Benoit andDonahue, 2003). The use of IMS may thus speed up the enrichment of the analyte.

This example describes a down-stream detection method using Campylobacter-specific bacteriophages, i.e., small viral organisms that attach to or infect living Campylobacter bacteria. Their attachment or infection is dependent of the phase of life cycle of the bacterium. Binding to or infection of Campylobacter may occur in the stationary, log or lag phase of the bacterium and depends on the phase species as well. Infection of the bacterium results usually in a high number of copies of the bacteriophage. Recording this increment of phages is, therefore, used as an analytical instrument to trace the presence of Campylobacter in the original sample.

The aim of this study is to demonstrate the application of bacteriophages as specific and sensitive analytical tools for the detection of Campylobacter in animal products, such as feces and (poultry) meats.

### 2. Materials and Methods

2.1 Experimental Set-Up

Following homogenization, e.g., facilitated by stomachering, IMS will be used to purify and concentrate Campylobacter from contaminated samples, such as meat and feces. In a second step, IMS-isolated bacteria will be incubated with an appropriate strain of bacteriophage. Non-attaching bacteriophages will be washed from the cell isolate using the same IMS procedure. Infected and/or bacteriophage-carrying IMS-immobilized Campylobacter are then introduced in a fresh and pure culture of reference Campylobacter that is in a stationary phase. This cell culture is used as a foreign host to boost the multiplication of the bacteriophages. Following a short culture to allow the bacteria to reach their log phase, bacteriophages will be harvested by centrifugation. The bacteriophage-containing supernatant will be incubated with LPS-coated fluorescent beads. Here, the bead is coated as described in the Example with the LPS isolated from Campylobacter used as the host organism. The presence of bacteriophages bound to the fluorescent beads will be tested in two ways. Following the addition of and incubation with anti-bacteriophage antibodies tagged with a fluorescent label, the amount of fluorescence will correspond with the concentration of bacteriophages and indirectly with the concentration of Campylobacter in the original sample. In an alternative approach, anti-LPS antibodies containing a fluorescent tag will compete with bacteriophages for binding places. A decrease of recorded fluorescence compared to a Campylobacter-free sample will, therefore, indicate a Campylobacter-positive sample.

The test will be validated in terms of selectivity and sensitivity for C. jejuni, C. coli and C. lari in different matrices, including feces, skin and meat from pigs and chickens. Closely related organisms, such as Arcobacter species, will be used to test the specificity of the method.

2.2 Bacterial and Viral Strains and Culture Conditions

C. jejuni (ATCC 33291) and C. coli (ATCC 33559) will be bought from Microbiologics (St. Cloud, USA). The bacteria will grow in tryptone soya broth (TSB) (Oxoid, CM 129, Hampshire, England) for 24 hours at 42° C., under microaerophilic atmosphere, which will be generated using a gas package (BBL, Becton Dickinson, Sparks, USA). Campylobacter are then plated onto Charcoal-Cefoperazone-Deoxycholate Agar (mCCDA) (Campylobacter blood-free selective a gar base (Oxoid, CM 739) with CCDA-selective supplement (Oxoid, SR155), cefoperazone 32 μg/ml and amphotericin B 10 μg/ml) and incubated under microaerophilic atmosphere for 24 to 48 hours at 42° C. One colony of pure Campylobacter is then transferred to tryptic soya agar (TSA) (Oxoid, CM131) and will be incubated under microaerophilic atmosphere for 24 to 48 hours at 42° C. and will then placed in a refrigerator at 4° C. until use. Campylobacter-infecting bacteriophages NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, —NTCC12683, and NTCC12684 are acquired from the National Type Culture Collection (London, United Kingdom).

2.3 Sample Preparation

The pure Campylobacter culture stored at 4° C. will be subcultured in TSB and incubated under microaerophilic atmosphere for 24 hours at 42° C. This is the host for exponential growth of the bacteriophage.

An amount of 25 g of ground chicken fillet will be suspended in 225 ml of Preston broth (Nutrient broth No. 2 (Oxoid, CM 67), 5% (v/v) lysed horse blood (Oxoid, SR48), Campylobacter growth supplement (Oxoid, SR232) and modified Preston. Campylobacter selective supplement (Oxoid, SR204)) contained by a stomacher bag. The Preston broth medium will be prepared according to the manufacturer's instructions. The sample-containing stomacher bag will be homogenized thoroughly for 90 seconds in a stomacher (Interscience, St. Nom, France). The entire suspension will then be incubated under microaerophilic atmosphere at 42° C. for an appropriate incubation time to allow growth of Campylobacter.

2.4 Immunomagnetic Separation

After enrichment with Preston broth, the stomacher bag containing the sample will be placed into the incubation pot of the IMS machine (Pathatrix™, Microscience, Cambridgeshire, UK). The apparatus is then operated according to the instructions of the manufacturer. Briefly, 50 μl of anti-Campylobacter magnetic beads (Microscience) will be added to the sample, which is then recirculated for 30 minutes at 37° C. The magnetically immobilized beads are released, washed with 100 ml of pre-warmed buffered peptone water (peptone (Becton Dickinson) 10 mg/ml, sodium chloride (Merck, Darmstadt, Germany) 5 mg/ml, disodium hydrogen phosphate dihydrate (Merck) 4.5 mg/ml, potassium dihydrogen phosphate (Merck) 1.5 mg/ml adjusted to pH 7.2) and then drawn to the magnet again. Wash solution was removed leaving a 200 μl suspension for selective growth and bacteriophage analyses.

2.5 Detection of Bacteriophages

Campylobacter-carrying IMS beads are contacted with a small volume of bacterium-specific bacteriophages. Following a short incubation to allow specific attachment of the phages to the surface of the targeted bacterium, IMS beads are washed and sampled to set a reference point in the final analysis procedure. The rest of the suspension is mixed with a suspension of fresh Campylobacter species to host the growing bacteriophage. Following incubation at 42° C., the suspension is centrifuged and the supernatant will be supplemented with a volume of Campylobacter LPS-coated fluorescent beads. Multiplication of the phages is then assessed following the addition of either fluorescently labeled anti-bacteriophage antibodies or fluorescently labeled anti-Campylobacter antibodies. Following an incubation of 15 minutes, the beads are analyzed using, e.g., a BioPlex device (Bio-Rad) to screen fluorescence immobilized on the beads as a result of specific binding reactions.

### Example 4

### Detection of Anti-Salmonella Antibodies in Porcine Serum and Meat Juice from Chickens Using Fluorescent Beads

### 1. Introduction

Microorganisms include a wide variety of bacteria, molds (fungi), parasites and viruses. Pathogenic microorganisms have attracted much attention from the public as consumers of contaminated food and water, which resulted in family or community outbreaks. As a consequence, the media and politicians have played their part in increasing consumer awareness and new legislation is in preparation or already in force.

With respect to pathogenic microorganisms, special attention is drawn to a number of zoonotic diseases, i.e., microbes transmissible from animals to human, for the following reasons: 1) most food- and water-borne diseases in human are zoonotic by nature; 2) many zoonotic agents have their transmission route through the environment, and 3) both contamination of food/water and environment are also used by (bio)terrorists to acquire maximum impact in the society.

Microbiological hazards can enter food chains at any point during pre-harvest, production, processing, transport, retailing, domestic storage or meal preparation. From their introduction on feed or food, highly complex environments can occur in which the microorganism can elude detection and inactivation. Efficient international distribution systems and rapid changes in consumer preferences can facilitate the swift penetration of pathogens through large populations, greatly shortening the reaction time available to public health agencies.

Authorities and food producers are convinced that rapid fast, versatile and selective (diagnostic) assays are needed for environmental, feed and food monitoring to react adequately to contaminated links in the food chain. A large portion of the explored monitoring techniques involved the use of affinity assay technologies, including biosensor platform.

In_{[AA5]} principle, detection of the presence of microorganisms can be carried out in two ways: directly or indirectly. In the direct assay, the organism itself is usually detected with the application of antibodies reacting with (sub)species- and/or strain-specific antigenic structures. This immunochemical analysis follows time-consuming sample preparation through culturing in selective growth media. In the case of parasite infections, this is not possible and direct detection involves microscopic inspection of samples. In the indirect assay, the presence of the microorganism is suggested by the detection of humoral (immunoglobulins) or cellular (e.g., cytokines) products of an immunological response of the infected host. In most studies, well-defined antigens are used to capture the host's immunoglobulins in any body fluid (serology). The observed binding then reveals the nature of an invasive infestation of a pathogen.

The advantages and disadvantages of indirect and direct pathogen detection are clear: i) individuals are not always immunologically responding to an infection; i.e., differences between low or high immune responders, ii) humoral responses are delayed several days or even weeks, possibly leaving a recent infection unnoticed, iii) serum antibodies can be found where the causative organism is not detected, as it has been rejected or retracted itself in certain (non-sampled) tissues, iv) serological investigations are very fast and offer better possibilities for high-throughput than direct detection, and v) serologic analysis of serum or plasma predicts the Salmonella infection status of a flock or herd better than direct antigen analysis, i.e., classical selective bacterial culturing.

In fact, serology outperforms direct, and in most cases insensitive, detection of tissue parasites, which can only be carried out by histochemistry or digestion techniques and microscopy. Significant differences are also apparent in sample collection and preparation. Bacteria, fungi and viruses have to be cultured from matrices to facilitate their detection in enriched solutions, but blood is relatively easily collected and prepared for analysis. Here, it should be noted, however, that antibodies cannot only be retrieved from blood, plasma or serum, but also from muscle (meat juice), milk, colostrums, cerebrospinal fluid and eggs. In particular, sampling of eggs, meat juice and/or milk is easier and more cost-effective than the sampling of blood, plasma, serum or cerebrospinal fluid.

Diagnostic methods based on serologic analysis of antibody-containing biological materials are, therefore, supportive in so-called logistic slaughtering of animals. In this innovative processing approach, evidence-based and reliable decisions are made on the basis of continuous and intensive monitoring at the farm level as to whether animals are allowed to enter a Salmonella-free or a Salmonella-contaminated processing infrastructure.

Among the components of the antigenic structure of the genus Salmonella, the somatic antigens are important as an instrument to trace immune response in animals upon an invasive infection of this organism. Somatic antigens are located on the polysaccharide part of lipid polysaccharide (LPS), which is a constituent of the bacterial cell wall. With detection of a humoral response with carefully chosen LPS, the identity of the serogroup of the infecting Salmonella can be deduced.

In Denmark, Germany, Greece and The Netherlands, 39.5% of all Salmonella-positive pigs sampled at the abattoir were determined as S. typhimurium. Dependent of country, other important isolates from pigs were S. derby (17.1%), S. infantis (8.0%), S. Panama (5.1%), S. ohio (4.9%), S. london (4.4%), S. livingstone (3.1%), S. virchow (2.7%), S. bredeny (2.1%), S. mbandaka (1.1%), S. Brandenburg (1.0%), S. goldcoast (0.8%).

In the case of chickens, 14% of the chickens were Salmonella-positive at flock level in 2002 in The Netherlands. The predominant serovar was in that case S. paratyphi B var. java. At the retail level, a comparable percentage (13.4%) was found in The Netherlands. The most frequent Salmonella serovars isolated from broilers in 14 EU member states were S. paratyphi B var. java (24.7%), S. enteritidis (13.6%), S. infantis (8.0%), S. virchow (6.7%), S. livingstone (5.7%), S. mbandaka (5.5%), S. typhimurium (5.3%), S. senftenberg (5.0%), and S. hadar (3.7%). S. paratyphi B var. java is dominating, but this is fully attributable to The Netherlands.

In food-producing chickens and swine, the prevalently occurring Salmonella serogroups thus belong to groups B, C and D, and in the case of swine, also E.

In this study, a new analytical affinity assay platform is explored for the indirect detection of Salmonella infection in pigs and chickens. This technology platform from Luminex analyzes internally coded beads that can be coated with different antigens in a single test. Only when both fluorescence of bead and bound analyte pass the detector, a response will be recorded. This approach is applied to detect anti-Salmonella antibodies in serum and meat drip.

### 2. Materials and Methods

2.1 Chemicals

Amine-coupling kits, consisting of N-hydroxysuccinimide (NHS), 1-ethyl-3-(3-dimethlylaminopropyl)carbodiimide hydrochloride (EDC) and ethanolamine hydrochloride-sodium hydroxide pH 8.5 were bought from Biacore AB (Uppsala, Sweden). Ethanol and trichloroacetic acid (TCA) were purchased from Merck (Darmstadt, Germany). Sodium cyanoborohydride and carbohydrazide were obtained from Fluka Chemie GmbH (Bucks, Switzerland). Porcine hemoglobin (Hb) was acquired from Sigma Chemical Company (St. Louis, Mo., USA). Water was obtained from of a MILLI-Q® water purification system (Millipore, Bedford, Mass., USA).

2.2 Materials

NAP-5 columns (0.5 ml; Sephadex G-25) were purchased from Amersham Biosciences and were used as described by the producer. CM5 biosensor chips were bought from Biacore AB. Dialysis bag (Spectra/Por) with a cut-off of 1 kDa was obtained from Spectrum Laboratories Inc. (Rancho Dominguez, Calif., USA). Alexa532 was from Molecular Probes (Leiden, The Netherlands). Goat anti-swine IgG (H+L) was ordered from Jackson Immunoresearch (West Grove, Pa., USA). This antibody was conjugated with Alexa532 using standard labeling procedures.

2.2.1 Anti-Salmonella Antisera

Salmonella monovalent “O” somatic monoclonal antisera against O4, O5, O6₁, O7, O8, O9, O10 were purchased from Sifin (Berlin, Germany). Antibody solutions were diluted in 50 mM PBS to their working concentrations.

2.2.2 Reference Avian and Porcine Sera

All reference sera were obtained from the Dutch Animal Health Service (Deventer, The Netherlands). The obtained avian reference sera were reactive with Salmonella enteritidis (serogroup D₁), S. typhimurium (serogroup B), S. pullorum/gallinarum (Spg; serogroup D₁) and S. infantis-(serogroup C₁), and were further referred to as CH-Se, CH-St, CH-Spg and CH-Si, respectively. These chicken sera were originally prepared for ELISA analyses as positive references. In addition, specific pathogen-free chicken serum (further referred to as CH-SPF) was purchased from this organization as a negative control reference sample. These sera were reconstitution from freeze-dried material by addition of water at a volume indicated by the producer. Likewise, porcine sera from animals challenged with S. typhimurium and S. livingstone (serogroup C₁) were acquired from GD and were referenced as P-St and P-Sl, respectively. In addition, Actinobacillus pleuropneumoniae serotype 2-reacting porcine serum (GD) used as control in a Complement Fixation Test, was exploited as negative control for porcine serum in the Salmonella biosensor assay.

2.3 Methods

2.3.1 Extraction of LPS_{[AAB6]}

Overnight cultures of Salmonella were prepared by applying 100 μl from their corresponding stocks on each of the 120 plates containing brain heart infusion agar (BHIa, Oxoid). The bacteria were harvested from the surface of the plates into 1 ml 9 g/l NaCl (saline) solution per agar plate using a trigalski spatula. Each plate was washed twice with 2 ml saline solution. Combined bacteria were centrifuged in six tubes at 10,000 g and 4° C. for 15 minutes and supernatant was discarded. This centrifugation step was repeated twice with 75 ml saline wash solution per tube each run. While kept on ice, pelleted bacteria were suspended in water at a volume ratio, which was five-fold to the weight of the bacteria. An equivolume of 0.250 M (Se) or 0.500 M (Sg, Sl, Sm and St) TCA was added to give end concentrations of 0.12 M and 0.25 M, respectively, followed by continuous stirring at 4° C. for three hours.

A lipopolysaccharide (LPS)-containing supernatant was then acquired at 20,000 g and 4° C. for 30 minutes. The pH of the supernatant was adjusted to pH 6.5 with 5 M sodium hydroxide and when nearing the aimed pH with 0.10 M sodium hydroxide. The final volume of the LPS-containing solution was determined prior to storage at −18° C. for 30 minutes. The solution was diluted with a double volume of freezing cold absolute ethanol from a −18° C. storage place, and incubation was continued overnight at −4° C. without stirring in a closed, in-house built device with circulating cold ethylene glycol/water (1:4, v/v). An LPS-containing pellet was obtained after centrifugation at 20,000 g and −4° C. for 30 minutes. The particulate material was suspended in a volume of 0.5 ml water per gram original bacterial mass weighed at the start of extraction process. The suspension was dialyzed in a 1-kDa dialysis bag against water at 4° C. for two days with regular intermittent refreshment of the water. The bag content was centrifuged at 20,000 g and at 4° C. for 30 min, and the supernatant was lyophilized. The lyophilisate was weighed to establish the recovery of LPS. LPS was reconstituted in water to make up an end concentration of 5 mg/ml. Dependent of type of LPS and batch number, a volume of 1 mg/ml porcine hemoglobin (Hb) was added to a concentration as indicated in the text. Each batch was portioned into 0.5-mg LPS fractions, which were dried using a vacuum evaporator and then stored at 5° C. to 8° C.

2.3.2 Oxidation of LPS_{[AAB7]}

A portion of 0.5 Mg hemoglobin-fortified LPS was dissolved in 500 μl mM sodium acetate pH 5.5. Following the addition of 20 μl mM sodium periodate (Sigma), the solution was incubated for 40 minutes on ice protected from light. The oxidation of LPS was quenched and the solution was desalted by passing 500 μl of the reaction mixture through a NAP-5 cartridge with a gravity-controlled flow. Modified LPS was eluted with 1 ml 10 mM sodium acetate, pH 4.0. Prior to use, the cartridge was conditioned thrice with 3 ml 10 nM sodium acetate, pH 4.0.

2.3.3 Immobilization of LPS_{[AA8]}

To immobilize the oxidized LPS-antigens to the beads, the carboxylic groups at the bead surface were activated with a mixture of EDC/NHS available from the amine-coupling kit for 20 minutes on a gyro rocker. Following centrifugation and removal of supernatant, activation was followed by a reaction with 5 mM aqueous carbohydrazide for 20 minutes. Beads with modified surface were pelleted again and upper liquid was discarded before addition of 1 M ethanolamine and incubation for 20 minutes. Following another centrifugation step at 14,000 g for 5 minutes, oxidized LPS dissolved in sodium acetate pH 4.0 was added to allow immobilization for 90 minutes. Following removal of the supernatant acquired through centrifugation, the linkage between bead-surface and antigen was stabilized using 100 mM sodium cyanoborohydride dissolved in 10 mM sodium acetate at pH 4.

2.3.4 BioPlex Assay

Following the warming-up of the bead counter device, this BioPlex (BioRad, Veenendaal, The Netherlands) was calibrated according to the instructions of the producer using a BioPlex calibration kit (BioRad). Samples were diluted in 50 mM PBS in wells of a microliter plate, which were then supplemented with 50 μL 5000 beads/mL LPS-coated beads. The antigen-antibody binding was allowed for 30 minutes on a microtiter plate shaker operated at 200 rpm. Then, 10 μL goat anti-swine IgG (H+L) tagged with Alexa532 fluorescent labels diluted eight times in 50 mM PBS were added and incubation was continued for 15 minutes on the shaker. Beads were then analyzed for their fluorescence profiles for 30 seconds on the BioPlex machine.

### 3. Results and Discussion

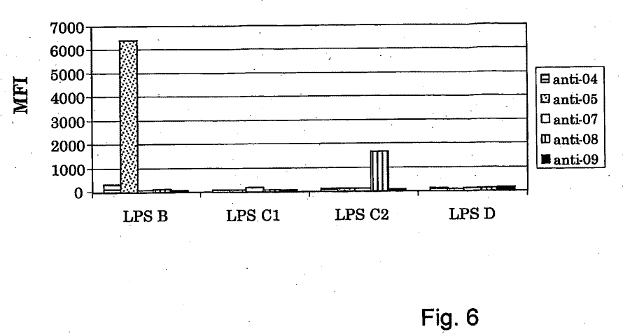

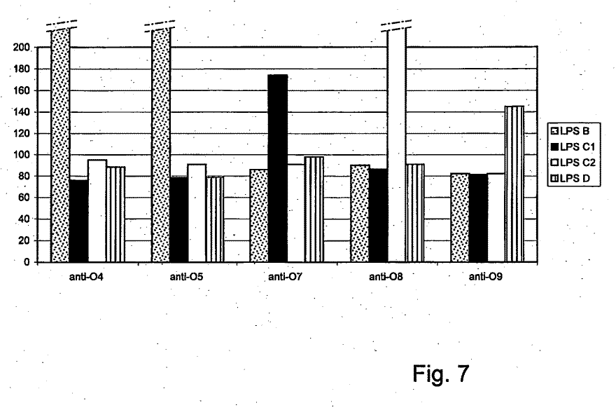

Fluorescent beads were prepared for coating with LPS from different specific Salmonella serovar sources representing serogroups B, C and D relevant as zoonoses in foods from chicken and swine. It should be noted that serogroup E, which is relevant for pork products, is not studied here. Following the immobilization of each type of LPS to individual beads, which are internally coded, the success of the coating was assessed using commercially available monoclonal antisera against somatic antigens O4, O5, O7, O8 and O9. However, while anti-O5 gave a response of 6398 units, anti-O9 gave 145 units, whereas the background signal of non-matching antigens-antibodies was less than 91 units in all cases (FIG. 6). In a similar way, anti-O4 and anti-O7 gave responses of 305 units and 174 units, respectively (FIG. 7). These differences in responses between commercially available antisera preparations were in very good correspondence with those observed using a surface Plasmon resonance (SPR) biosensor and reflect differences in antibody titers.

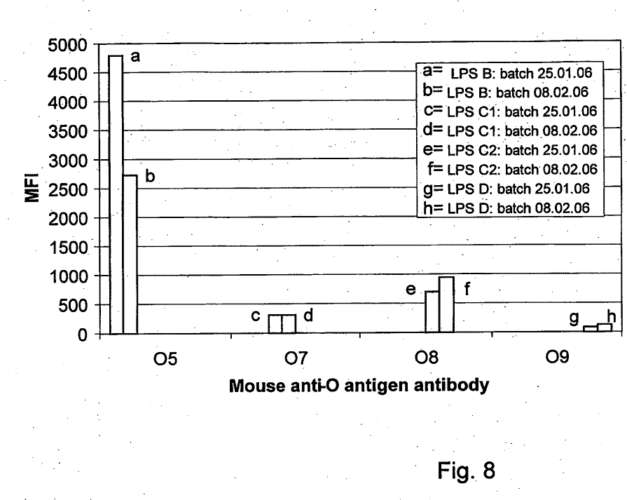

In a similar way, the activity of identical LPS batches oxidized on different days were tested using a similar panel of commercial antisera (FIG. 8). Compared to the other oxidation batch, responses ranged between 57% and 148%, which is, susceptible for improvements.

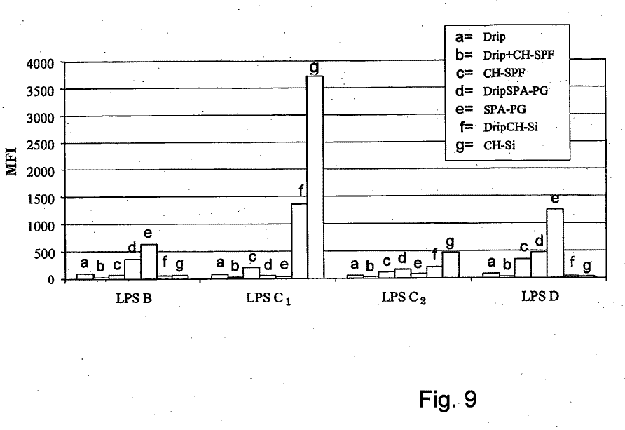

Different preparations of meat drip, i.e., juice that is acquired from muscle tissue following a freeze and thaw cycle, and sera from chickens were analyzed (FIG. 9). Recorded activities were as expected. Meat drip, serum and a mixture of meat drip and serum from Salmonella-free chickens gave low abundant fluorescent conjugated beads. In contrast, anti-S. pullorum and anti-S. gallinarum should give a response on serogroups B and D, as it contains antigens O1 and O12, which it does for drip and serum. S. infantis contains antigen O6, which is shared by C₁ and C₂. Indeed, this activity is observed in drip and serum_{[AAB9]}.

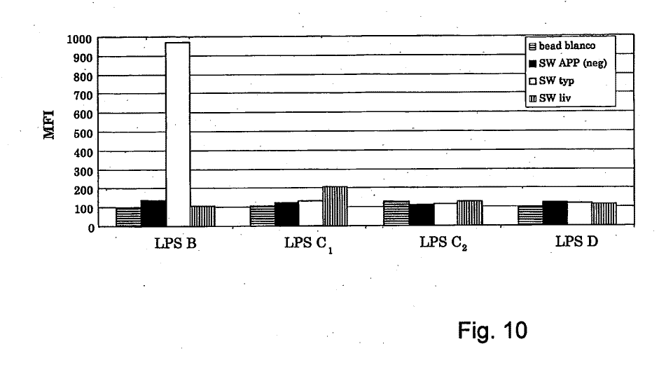

Besides chicken serum, prepared swine serum were tested as well (FIG. 10). Serum from Salmonella-free pigs gave MFI responses in the range of 110 units (serogroup C₂) to 137 units (serogroup B) and were close to the responses of beads only incubated with buffer, namely from 94 units (serogroup D) to 129 units (serogroup C₂). As expected, significant signals were recorded when sera were spiked with anti-S. typhimurium and S. livingstone antisera, namely 969 units on serogroup B and 207 units on serogroup C₁, respectively. The spiked sera did not react with non-corresponding antigens giving responses between 104 MFI units and 131 MFI units.

### Example 5

### Determination of Anti-Salmonella Antibodies in Exotic Avian Species Using an SPR Biosensor_{[AAB10]}

### 1. Introduction

1.1 Aim of Study

The aim of this investigation was to explore whether the developed SPR biosensor technology based on the use of immobilized selected Salmonella LPS to indirectly detect Salmonella infections in food-producing animals, is able to detect such infections in exotic animal species as well.

### 2. Materials

Plasma from tocotoucans (Rhamphastos toco) and a sharp-tailed grouse (Tympanuchus phasianellus), which were infected with S. typhimurium of different phage-types, were kindly provided by Dr. W. Schaftenaar and Ing. M. de Boer (Veterinary Department, Rotterdam Zoo, The Netherlands). The disease history of these animals is the following.

From the feces of a Tocotoucan sampled on Mar. 24, 1994, S. typhimurium phagetype 292 was isolated. From another feces sample of the same bird, —S. typhimurium phagetype 352 was isolated on Jun. 28, 1994. On Aug. 24, 1994, plasma was collected from this animal and used for SPR analysis in this study.

Blood was collected from a diseased sharp-tailed grouse on Oct. 28, 1997. The plasma prepared from this blood was used for analysis in this study. This animal died the next day. S. typhimurium phagetype 507 was isolated from the dead bird.

### 3. Methods

An SPR biosensor (Biacore 3000) containing a sensor chip of which flow channels were coated with LPS from S. enteritidis, S. livingstone, S. goldcoast and S. typhimurium, was operated as described earlier. Plasma samples were diluted as described for sera in Examples 1 and 2 and analyzed.

### 4. Results and Discussion

The invention was developed in the first place for application in the food chains to secure the safety of food of animal origin with respect to Salmonella contaminations. Nonetheless, the field of applicability was tested with plasma collected from two exotic avian species, namely a tocotoucan (R. toco) and a sharp-tailed grouse (T. phasianellus), which were infected with S. typhimurium as disclosed by classic microbiological diagnostics (personal communication with M. de Boer, Blijdorp Zoo, Rotterdam).

The feces of the tocotoucan was found positive five and two months before blood was sampled and a humoral response could develop over a relatively long period of time. Indeed, the biosensor response was high (Table 33). Compared to blank serum from SPF chickens and to standard antiserum (anti-serogroups A to S), the reactivity with S. typhimurium LPS was dramatically high (4185 response units (RU)). A response was also observed on the channel of S. enteritidis (1220 RU), which was also observed for serum from chickens exclusively highly infected with S. typhimurium and is in accordance with the presence of somatic antigen O12 in both serovars and thus in serogroups B and D (cf. Tables 1 and 3). Unexpectedly, a relatively high response was also observed at the S. goldcoast channel. It cannot be excluded here that this bird was infected with multiple Salmonella serovars simultaneously or sequentially with S. typhimurium as the last infection, including a C₂ infection.

The plasma of the sharp-tailed grouse was not very reactive with the different LPS types, but reactivity was in all cases higher than that of the blank serum and on Sl and St, LPS was higher than that of the reference antiserum (Table 33). As the bird died rapidly from the infection, a significant humoral response against the selected antigens was probably not fully developed and not detected by the biosensor. It should be noted that serology is, therefore, not very suitable for diagnosis on an individual level. As evidenced by Swanenburg (Utrecht Thesis, Utrecht University, 2000), serology is, in particular, suitable for assessing the Salmonella status on a population level.

TABLE 33
Results of the analysis of plasma collected from a tocotoucan and a
sharp-tailed grouse that were infected with S. typhimurium. The results are
expressed in relative response units. Samples were analyzed thrice.

| Flow | | | | | |
|---|---|---|---|---|---|
| channel | Immobilization | | | | Plasma from |
| coated with | level of probing | Serum from | Anti- | Plasma from | sharp-tailed |
| LPS from | LPS | SPF^{a} chickens | serogroup B^{b} | tocotoucan | grouse |
| Se | 3241 | 17 (3) | −3 (1) | 1220 (304) | 33 (1) |
| Sg | 3354 | 9 (4) | −8 (1) | 492 (186) | 59 (9) |
| Sl | 5241 | 6 (6) | −29 (2) | 58 (12) | 89 (8) |
| St | 2755 | 46 (6) | 663 (33) | 4185 (362) | 73 (8) |

^{a}serum from specific pathogen-free chickens, i.e., blank serum
^{b}commercially available monoclonal antibody reacting with Salmonella serogroup B.

### Example 6

### Detection of Bacteriophage Felix O1 (FO1) Through its Binding to Salmonella LPS Immobilized on an SPR Biosensor Chip Surface

### 1. Goal

To determine the binding of the bacteriophage FO1 to LPS immobilized to the biosensor surface.

### 2. Approach

To prove binding of bacteriophage Felix O1 (FO1, Felix d'Hérelle Reference Centre for Bacterial Viruses, Laval, Canada) to Salmonella LPS, the bacteriophage was diluted in HBSEP to obtain a concentration series. These samples were injected for two minutes on the biosensor to allow binding to LPS from S. typhimurium, S. enteritidis, S. goldcoast and S. livingstone, which were each immobilized separately on an individual flow channel of the sensor chip.

### 3. Results

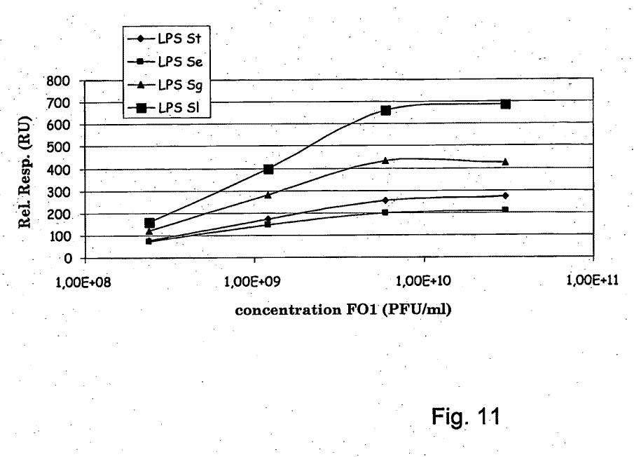

The results are summarized in FIG. 11.

Although a relatively high concentration of bacteriophage was needed to obtain a significant response, it is evident from this experiment that 10⁹ PFU of FO1 bacteriophages/mL and higher bound to LPS coupled to the chip surface.

### Example 7

### Detection of Bacteriophage FO1 Through its Binding to S. Typhimurium LPS Immobilized on an SPR Biosensor Following its Incubation with S. Typhimurium, S. Enteritidis, S. Goldcoast and S. Livingstone

### 1. Goal

To determine the binding of bacteriophage FO1 to a live culture of Salmonella spp. in HBSEP.

### 2. Approach

Different concentrations of cultures of S. typhimurium, S. enteritidis, S. goldcoast, S. livingstone and blank medium were mixed for 5 minutes with 1.2×10⁹ PFU bacteriophage. FO1. After incubation, bacteria were spun down and supernatant was analyzed on the Biacore with a biosensor chip containing immobilized LPS from S. typhimurium.

### 3. Results

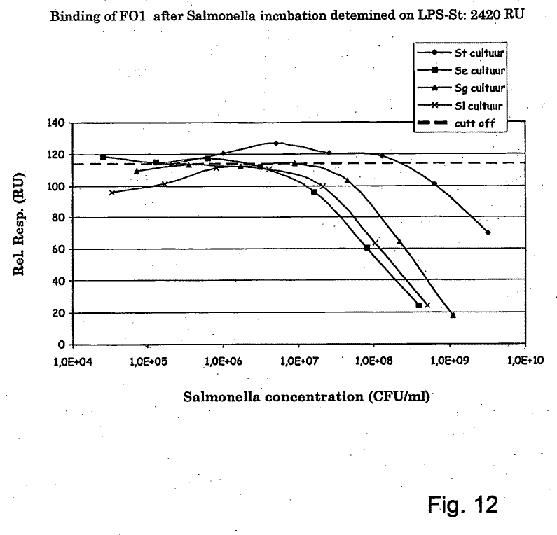

In order to determine significant responses, a cut-off value was established by the averaged readings of blank medium containing no Salmonella but 1.2×10⁹ PFU bacteriophage, minus three times standard deviation. Applying this value disclosed that Salmonella should be present at a concentration of at least 6×10⁸ CFU/mL, 3×10⁶ CFU/mL, 4×10⁷ CFU/mL and 3×10⁴ CFU/ml for S. typhimurium, S. enteritidis, S. goldcoast, S. livingstone, respectively, to give a significant response (FIG. 12).

### 4. Discussion

The absorption rate of bacteriophage FO1 to Salmonella spp. is probably dependent of the accessibility of N-acetylglucosamine in the core region, the binding site of FO1 (Lindberg, 1977; Lindberg and Holme, 1969_{[AAB11]}). Long and numerous O-side chains occurring in the polysaccharide region of the LPS of targeted Salmonellae could, therefore, impair the binding of FO1 to the analyte. It is, therefore, expected that free bacteriophages will have a variable binding capacity towards. Salmonella strains and that propagation of the virus will largely depend on the molecular profile of the exposed LPS.

When immobilizing LPS-protein complexes at the surface of a solid carrier for a diagnostic method, as described in the invention, a dense network of ligands may be formed. In the presented Biacore analysis, the density of the complex and the hindrance by the proteins may play a role in the observed difference in bacteriophage binding to the four LPS types.

It should be noted here that, as discussed in the invention as well, oxidation of monosaccharide constituents in the core region is expected, including that of the N-acetylglucosamine. The ligand for the bacteriophage is, therefore, affected and this may influence the sensitivity of the test.

### Example 8_{[AAB12]}

### Propagation of Bacteriophage FO! in Salmonella and Non-Salmonella Strains

### 1. Goal

The propagation of FO1 is not exclusive in Salmonella spp., but possibly also in non-Salmonella strains (3). This study was initiated to investigate the selectivity of the proliferation of the bacteriophage FO1. For this purpose, a number of important food non-Salmonella pathogens were exposed to the FO1 bacteriophage.

### 2. Methods

Seven non-Salmonella strains (Listeria monocytogenes, Escherichia coli, Pseudomonas aeroginosa, Bacillus cereus, Citrobacter, Enterococcus feacalis, and Staphyllcoccus aureus) and seven Salmonella strains (S. cholerasuis, S. berta, S. meleagridis, S. Agona, S. pullorum, S. Virchow and S. enteritidis) were grown in Tripton Soy Broth (Oxoid CM129).

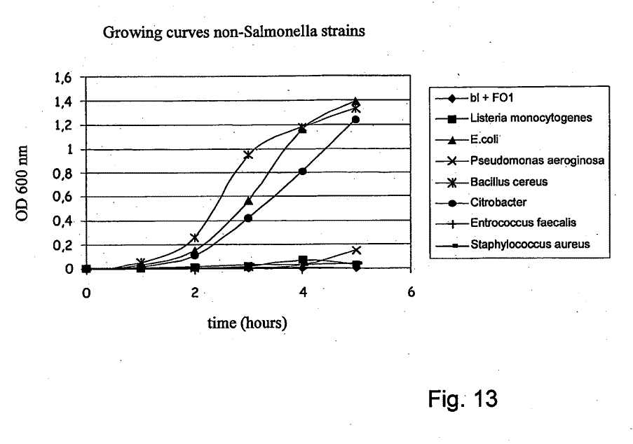

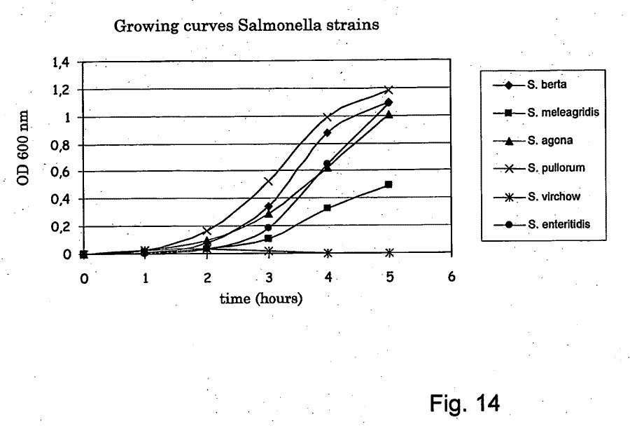

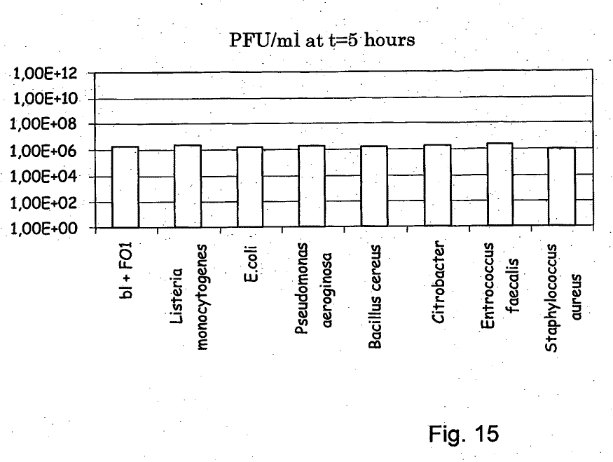

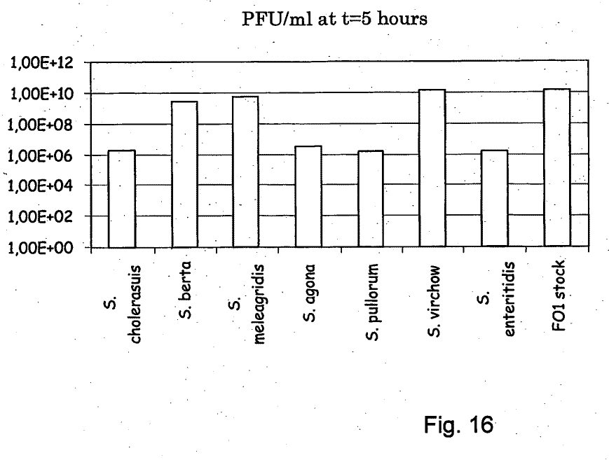

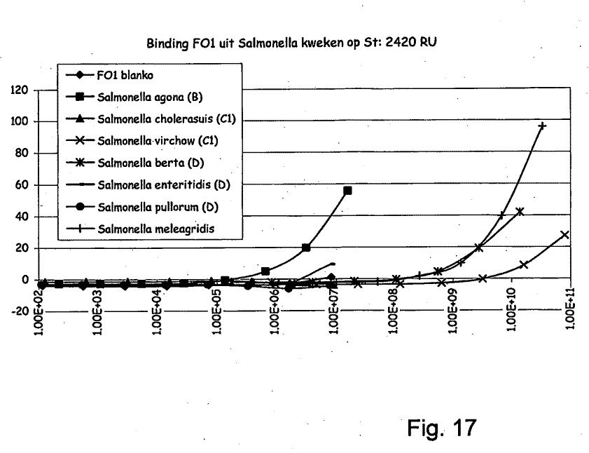

At t 0 hours, 1.2×10⁸ PFU of FO1 (end concentration 1×10⁶ PFU/ml) was added to all cultures. Every hour, a sample was drawn, and absorbance at λ 600 nm (FIGS. 13 and 14), plaque-forming units/ml (FIGS. 15 and 16) and, after concentration and buffer exchange, binding to immobilized St-LPS immobilized on a sensor chip surface in a Biacore biosensor (FIG. 17), were determined.

Some non-Salmonella bacteria, including L. monocytogenes, P. aeroginosa, E. faecalis and Staph. aureus, did not show growth in five hours of culture (FIG. 13). These bacteria were obviously not loaded nor infected by the bacteriophage FO1 because the concentration of bacteriophages did not rise over time and was stationary at 1×10⁶ PFU/ml (FIG. 15).

In contrast, all Salmonella serovars, except S. virchow, grew in five hours of incubation (FIG. 14). This S. virchow strain was probably lysed completely by the proliferating bacteriophages, as a clear increase in the concentration of bacteriophages can be shown from 1×10⁶ PFU/ml to 1×10¹⁰ PFU/ml (FIG. 16). In a similar way, S. berta and S. meleagridis bacteria showed an increase of bacteriophage concentration. However, these bacteria, in particular, S. berta, showed good growth (FIG. 14).

In the context of the invention, the binding of propagated bacteriophages to the sensor surface is of greatest interest. For this purpose, bacteriophages propagated in Salmonella were concentrated, dialyzed and serially diluted before SPR biosensor analysis (FIG. 17). Unexpectedly, comparable bacteriophage concentrations gave dramatic different biosensor responses. Most probably, phages were lost, in particular during the concentration and dialysis step, during the sample preparation. Nevertheless, the preparations of bacteriophages, which have shown propagation (cf. FIG. 16), clearly gave higher responses than the blank sample, which contained the starting concentration of bacteriophages only.

### 3. Conclusion

These experiments showed that bacteriophages can be used as an analytical tool to detect the presence of Salmonella in samples and that Salmonella LPS immobilized to a solid surface can be used to probe the increment of the bacteriophages as a result of propagation of the virus in the host bacteria following a short incubation period.

### Example 9

### Immobilization of Salmonella-derived LPS onto fluorescent beads (Luminex) and Detection of Antibodies Reporting a Current or Past Salmonella Infection in Various Biolopical Samples_{[AAB13]}

WARNING AND SAFETY PRECAUTIONS: Lipid polysaccharides are potent immunogens, which can bring sensitive persons into a septic shock upon intake or inhalation. Precautions should be made to prevent contact with this biochemical.

Sodium periodate is an oxidizing agent and may cause explosions when brought into contact with strong reducing agents.

The procedure utilizes sodium cyanoborohydride. The procedure should, therefore, be carried out with precautions as with, for example, hand gloves and a mask. Use this chemical substance only in a chemical fume hood. The material is very toxic to aquatic organisms and may cause long-term adverse effect in the aquatic environment. The material and solution waste should be disposed of as hazardous waste.

### 1. Introduction

Among the components of the antigenic structure of the genus Salmonella, the somatic antigens are important as an instrument to trace immune response in animals upon an invasive infection of this microorganism. Somatic antigens are located on the polysaccharide part of lipid polysaccharide (LPS), which is a constituent of the bacterial cell wall. After extraction and isolation of LPS from carefully chosen Salmonella serotypes (cf. SOP CHEMIE/A21_{[AAB14]}), antigen-containing LPS is coupled covalently to beads, which are internally coded by a specific mixture of fluorescent material. The exploited technology platform from Luminex can identify-up to 100 differently internally coded beads in a single test. A single species of beads can be immobilized with a mixture of different LPS-reflecting serogroups, or different species of beads can be each immobilized with LPS reflecting a single specific serogroup or serovar of the pathogenic microorganism. The LPS-containing beads are incubated with body-derived materials, such as blood, plasma, serum, meat drip/juice, egg yolk, milk, etc., to enable anti-Salmonella antibody-antigen binding. The specific binding is detected following a second incubation with fluorescently tagged anti-immunoglobulin antibodies in a device simultaneously analyzing the emission wavelengths of excitated beads and tagged antibodies. Only when both fluorescence of bead and antibody are detected simultaneously, a response to a specific bead species will be recorded.

This SOP describes the method for oxidation, immobilization of LPS onto beads (Luminex) and an assay to assess the quality produced.

### 2. Scope and Field of Application

To analyze biological fluids, such as serum samples from chicken and pigs, for the presence of anti-Salmonella antibodies reacting with O3, O4, O5, O6, O7, O8, O9, O10 and O12 somatic antigens reflecting a history of or current infection of Salmonella from serogroups B, C, D and E.

### 3. References

Extraction and isolation of lipopolysaccharides from Salmonella spp.; Immobilization of Salmonella-derived LPS onto a biosensor chip (Biacore) and detection of serum antibodies reporting a current or past Salmonella infection; optimalization of protein addition to LPS for immobilization and detection of serum antibodies; see Example 1.

### 4. Definitions

c=concentration in % (m/v), % (v/v), mol/l or mmol/l as indicated.

### 5. List of Abbreviations

5.1 EDC, 1-ethyl-3-(3-dimethylaminopropyl) Carbodiimide Hydrochloride

5.2 LPS, Lipopolysaccharides

5.3 NaCl, Sodium Chloride

5.4 NaOH, Sodium Hydroxide

5.5 NHS, N-Hydroxysuccinimide

5.6 PBS, Phosphate-Buffered Saline

5.7 Se, Salmonella enteritidis

5.8 Sg, Salmonella goldcoast

5.9 Sl, Salmonella Livingstone

5.10 Sm, Salmonella meleagridis

5.11 SOP, Standard Operating Procedure

5.12 St, Salmonella typhimurium

5.13 TCA, Trichloric Acetic Acid

### 6. Principle

LPS is oxidized in the presence of a protein facilitated by sodium periodate. The LPS-protein solution is desalted using a NAP-5 column. After activation of the carboxylic acid groups at the surface of the beads with the aid of 1-ethyl-3-(3-dimethylaminopropyl) carbodiimide hydrochloride (EDC), N-hydroxysuccinimide (NHS) followed by a reaction with carbohydrazide, desalted oxidized LPS-protein complex is immobilized to the solid phase of the beads. Bound LPS is then stabilized with sodium cyanoborohydride. Prior to routine use, the performance of bead-conjugated LPS to bind anti-Salmonella antibodies is assessed using a panel of reference monoclonal agglutination sera.

### 7. Reactions

7.1 Oxidation of Carbohydrate Moiety

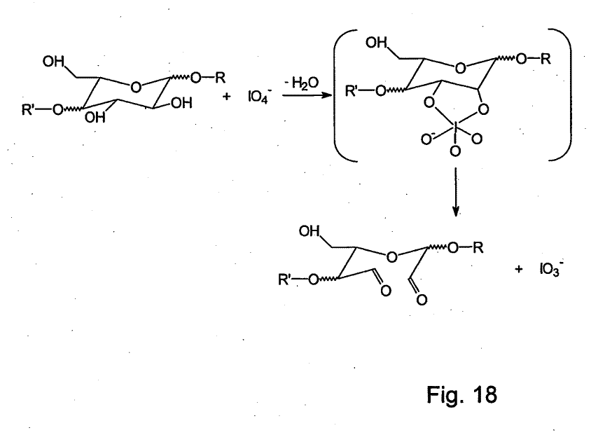

Periodate will induce an oxidative disruption of linkages between vicinal cisdiols on, in particular, carbohydrate moieties, as in, e.g., mannose, to yield aldehyde functionalities, see FIG. 18. This reaction is typically performed in buffers at a pH range between 4.5 and 5.5 in the dark using a freshly prepared 10 to 100 mM sodium meta-periodate in 0.1 M sodium acetate.

NOTE 1: The positions of conjugations indicated in FIG. 18, to link up the depicted monosaccharide with other monosaccharide residues in a polysaccharide, as in, e.g., LPS, are just given here as an example. R′ and R indicate the distal and the proximal positions, respectively, in the carbohydrate chain. The oxidation of hydroxyl groups into aldehydes may repeat itself within the polysaccharide chain in each monosaccharide constituent containing susceptible vicinal diols.

NOTE 2: Periodate will also oxidize, when present, certain β-aminoethanol derivates, such as the hydroxylysine residues in collagen, as well as methionine (to its sulfoxide) and certain thiols (usually to disulfides). In addition, N-terminal serine and threonine residues of peptides and proteins can be selectively oxidized by periodate to aldehyde groups. These reactions, however, usually occur at a slower rate than oxidation of vicinal diols.

7.2 Conjugation to Protein

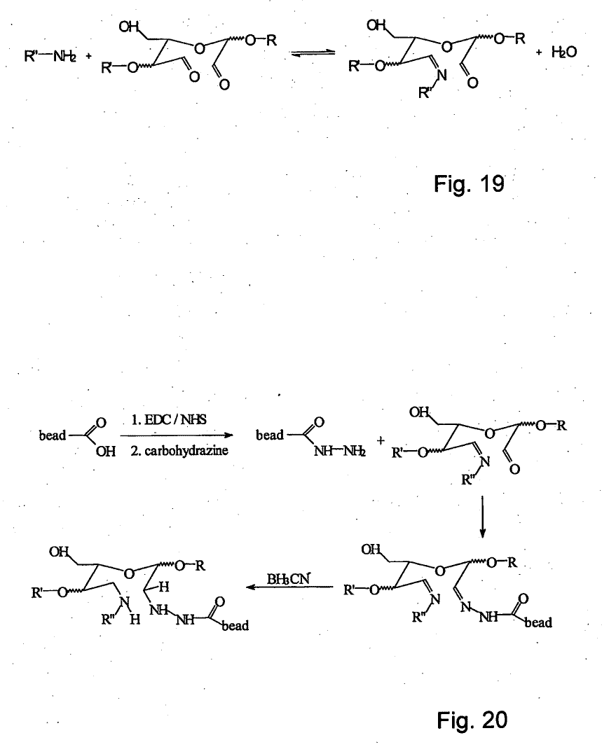

Oxidation is performed in the presence of a protein. The bis-aldehyde compounds, such as the oxidized monosaccharide constituents in the polysaccharide chain of LPS here, may react with any amino group in a protein and may form a Schiff-based linkage resulting in a substituted imine. See FIG. 19.

NOTE: The substituted imine is stabilized while the complex is attached to the bead surface, by a reduction facilitated by cyanoborohydride. This type of reaction scheme is known as a reductive amination.

7.3 Immobilization to Fluorescent Beads

The carboxylic acid-labeled beads are activated using N-ethyl-N′-(3-dimethyl aminopropyl)-carbodiimide hydrochloride (EDC) and N-hydroxysuccinimide (NHS). The activation is followed by a reaction with carbohydrazine. The reactive aldehyde functionalities react spontaneously with the hydrazide to hydrazones, which are then reduced to stabilize the covalent bonds. See FIG. 20.

NOTE: The protein (R″) carries multiple —NH₂ groups and can, therefore, be conjugated with multiple oxidized LPS entities. On the other hand, the polysaccharide part in LPS may carry multiple free aldehyde groups in a single molecule. These aldehyde groups may be, for a part or completely, captured by the hydrazide layer on the beads. The net result may be a very stable complex network of protein-LPS covalently linked to the bead surface.

### 8. Reagents and Materials

In the complete procedure, only reagents of recognized analytical grade and only distilled water or water of equivalent purity are used, unless stated otherwise. Reference to a company is for information and identification only and does not imply a recommendation unless so stated.

8.1 Chemicals
8.1.1 Acetic acid (J. T. Baker, Deventer, The Netherlands)
8.1.2 Amine coupling kit (Biacore AB, Uppsala, Sweden) consisting of:
   8.1.2.1 Vial containing 115 mg N-hydroxysuccinimide (NHS)
   8.1.2.2 Vial containing 750 mg 1-ethyl-3-(3-dimethlylaminopropyl) carbodiimide hydrochloride (EDC)
   8.1.2.3 Vial containing 10.5 ml, c=1 mol/l, ethanolamine hydrochloride—sodium hydroxide pH 8.5
8.1.3 Bio-Plex Calibration kit (Bio-Rad, Veenendaal, the Netherlands)
8.1.4 Carbohydrazide, CN₄H₆O (Fluka Chemie GmbH, Buchs, Switzerland)
8.1.5 Carboxymethyl-dextran sodium salt (Fluka)
8.1.6 Potassium dihydrogen phosphate (KH₂PO₄) (Merck, Darmstadt, Germany)
8.1.7 Proclin 150 (Supleco, Bellefonte, Pa., USA)
8.1.8. Monoclonal anti-Salmonella O-antigens:
   8.1.8.1 anti-O4 (SIFIN, Berlin Germany)
   8.1.8.2 anti-O5 (SIFIN)
   8.1.8.3 anti-O6, (SIFIN)
   8.1.8.4 anti-O7 (SIFIN)
   8.1.8.5 anti-O8 (SIFIN)
   8.1.8.6 anti-O9 (SIFIN)
   8.1.8.7 anti-O10 (SIFIN)
8.1.9 Salmonella LPS, in-house isolated LPS by TCA extraction (SOP CHEMIE/A21) prepared from the Salmonella bacteria serovars enteriditis (Se), goldcoast (Sg), livingstone (Sl), meleagridis (Sm) and typhimurium (St) with protein (SOP CHEMIE/A23)
8.1.10 Sheep anti-mouse Ig-PE (Chemicon, Boronia, Victoria, Australia)
8.1.11 Sodium acetate trihydrate (J. T. Baker, Phillipsburgh, N.J., USA)
8.1.12 Sodium chloride (Merck)
8.1.13 Sodium cyanoborohydride (NaCNBH₃) (Fluka)
8.1.14 di-Sodium hydrogen phosphate (Na₂HPO₄) (Merck)
8.1.15 Sodium hydroxide, c=50 mmol/l (Biacore)
8.1.16 Sodium m-periodate (NaIO₄) (Sigma-Aldrich, Zwijndrecht, the Netherlands)
8.1.17 Water is obtained from a MILLI-Q® water purification system

8.2. Solutions
8.2.1 Acetic acid solution, c=0.1 g/ml: Dilute 1 ml acetic acid (8.1.1) with 9 ml water.
8.2.2 Acetate buffer solution, c=10 mmol/l, pH 4.0: dissolve 0.272 g sodium acetate trihydrate (8.1.11) in 180 ml water and adjust to pH 4.0 with acetic acid (8.1.1) and make up to 200 ml with water. This buffer is stable for approximately six months.
8.2.3 Acetate buffer solution, c=1.0 mol/l, pH 5.5: dissolve 13.6 g sodium acetate trihydrate (8.1.11) in 90 ml water and adjust to pH 5.5 with acetic acid. (8.1.1) and make up to 100 ml with water. This buffer is stable for approximately six months.
8.2.4 Acetate buffer solution, C=100 mmol/l, pH 5.5: dilute 1.0 ml acetate bluffer solution, c=1.0 mol/l (8.2.3) with 9.0 ml water.
8.2.5 Carbohydrazide solution, c=100 μmmol/l: dissolve 9.0 mg carbohydrazide (8.1.4) in 1000 μl water.
8.2.6 Carbohydrazide solution, c=5 mmol/l: dilute 10 μl carbohydrazide solution (8.2.5) with 190 μl water. Prepare just before use.
8.2.7 EDC-solution: reconstitute EDC (8.1.2.2.) in 10.0 ml water.
   8.2.7.1 Fractionate 100-μl aliquots of this solution (8.2.7) in polypropylene tube (9.15). Store at −18° C. or at lower temperature. The aliquots are stable for two months. Before use: thaw frozen aliquots and agitate them gently to ensure homogeneous solutions.
8.2.8 Ethanolamine solution: Pipette 200 μl c=1 mol/l ethanolamine solution (8.1.2.3) in a polypropylene tube (9.15).
8.2.9 NHS-solution: reconstitute NHS (8.1.2.1) in 10.0 ml water.
   8.2.9.1 Fractionate 100-μl aliquots of this solution (8.2.9) in polypropylene tube (9.15). Store at −18° C. or at lower temperature. The aliquots are stable for two months. Before use: thaw frozen aliquots and agitate them gently to ensure homogeneous solutions.
8.2.10 PBS (5.6), c 100 mmol/l, pH 7.2: dissolve 67.9 g sodium chloride
(8.1.12), 14.7 g di-sodium hydrogen phosphate (8.1.14) and 4.3 g potassium dihydrogen phosphate (8.1.6) in 1 L water (8.1.17).
8.2.11 PBS, c=10 mmol/l, pH 7.2: dilute 100 mL ten times concentrated PBS (8.2.10) in 1 L water (8.1.17).
8.2.12 Anti-mouse Ig-PE, prediluted: dilute fluorescent conjugate five times by mixing 40 μl anti-mouse Ig-PE (O) with 160 μl PBS (8.2.11).
8.2.13 Sodium cyanoborohydride, c=1.00 mol/l: dissolve 62.8 mg sodium cyanoborohydride (8.1.13) in 1000 μl 10 mM acetate solution pH 4.0 (8.2.2).
8.2.14 Sodium cyanoborohydride, c=100 mmol/l: dilute 180 μl sodium cyanoborohydride solution (8.2.13) with 1620 μl acetate solution, c=10 mmol/l, pH 4.0 (8.2.2). Prepare just before use.
8.2.15 Sodium hydroxide, c=5 mmol/l: Dilute 400 μl sodium hydroxide, C=50 mmol/l (8.1.15) with 3.6 ml water in glass vial (9.10).
8.2.16 Sodium m-periodate solution, c=100 mmol/l: Add to 214 mg sodium m-periodate (8.1.16) 10.0 ml water (8.1.17).
8.2.17 Sodium m-periodate “ready to use”: Pipette 100 μl of sodium m-periodate solution, c=100 mmol/l (8.2.16) in a 1.4 ml polypropylene tube (9.14) and dry with a centrifugal evaporator (9.4).
8.2.18 Sodium periodate solution, c=50 mmol/l: Dissolve sodium periodate “ready to use” (8.2.17) in 200 μl acetate solution, c=100 mmol/l pH 5.5 (8.2.4). Prepare just before use.

8.3 Standard Monoclonal Reference Solution
8.3.1 Concentrated monoclonal Salmonella anti-O5 (8.1.8.2): dilute 5 μl anti-O5 ten times by adding 45 μl PBS, c=10 mmol/l, pH 7.2 (8.2.11).
8.3.2 Monoclonal Salmonella anti-O5: 7.5 μl anti-O5 (8.3.1) ten times diluted by addition of 67.5 μl PBS, c=10 mmol/l, pH 7.2 (8.2.11).
8.3.3 Monoclonal anti-Salmonella O-antigens (8.1.8): dilute 7.5 μl of each monoclonal (8.1.8.1, 8.1.8.3, 8.1.8.4, 8.1.8.5, 8.1.8.6, 8.1.8.7) with 67.5 μl PBS (8.2.11) in a microtiter plate (9.20).
8.3.4 Thrice diluted monoclonal antibodies: add 25 μl monoclonal antibody solution (8.3.2 and 8.3.3) to 50 μl PBS, c=10 mmol/l, pH 7.2 (8.2.11) in wells of the same microtiter plate (8.3.3).
8.3.5 Repeat step 8.3.4 twice to obtain 9 and 27 times diluted antibodies in fresh wells of the microtiter plate in the case of anti-O4, anti-O6, anti-O7, anti-O8, anti-O9 and anti-O10. In the case of anti-O5, these dilution factors were 90 and 270 times, respectively.
8.3.6 Remove 25 μl from the highest dilution (8.3.5).
8.3.7 The antibody solutions are now ready for use.

NOTE: The final dilution factors are 100, 300, 900 and 2700 times in the case of anti-O5, whereas in the other cases, antibodies were finally diluted 10, 30, 90 and 270 times compared to the original preparation (8.1.8).

8.4 Auxiliary Materials
8.4.1 NAP-5 column (0.5 ml, Sephadex G-25, Amersham Biosciences).
8.4.2 COOH-Beads (5.6 μm COOH microspheres) numbers 24, 25, 26, 27 and 28 mixed in 0.01% aqueous merthiolate at 1.25×10⁷ beads/mL-(BioRad).

### 9. Equipment and Apparatus

Reference to a company is for information and identification only and does not imply a recommendation unless so stated. Equivalent equipment and apparatuses may be as appropriate as well.
9.1 Analytical balance (type AE 240, Mettler, Zurich, Switzerland)
9.2 BioPlex 200 (Bio-Rad)
9.3 Bürker-Türk counting chamber (Mainit B. V., Wormer, The Netherlands)
9.4 Centrifugal evaporator (Jouan, Saint-Herblain, France)
9.5 Finn pipette, 5-40 μl (Labsystems Oy, Helsinki, Finland)
9.6 Finn pipette, 40-200 μl (Labsystems Oy)
9.7 Finn pipette, 200-1000 μl (Labsystems Oy)
9.8 Finn pipette, 1-5 ml (Labsystems Oy)
9.9 Glass collection tube, 5 ml, stoppered (Renes, Zeist, The Netherlands)
9.10 Glass vial diameter 16 mm (Sarstedt B. V., Etten-Leur, the Netherlands)
9.11 Gyro rocker SSL3 (Stuart, Barloworld Scientific Ltd. Stone, Staffordshire, United Kingdom)
9.12 Microscope Axiovert 25 (Zeiss, Sliedrecht, the Netherlands)
9.13 Microscope glass cover slides (Chance Propper Ltd., Smethwick, Warley, England)
9.14 Mini shaker with microtiter cup head (MS 1, Janke & Kunkel, Staufen, Germany)
9.15 Polypropylene, tube, diameter 7 mm, with snap caps (Biacore)
9.16 Polypropylene tube, 1.4 ml (Micronic, Lelystad, The Netherlands)
9.17 pH meter (type pH 537, WTW, Weilheim, Germany)
9.18 Sonification bath, Branson 2200 (Branson Ultrasonics B.V., Soest, The Netherlands)
9.19 Vacuum manifold, to run several NAP-5 columns simultaneously (type SPE-12G, with PTFE stopcock(s), J. T. Baker)
9.20 V-bottomed microtiter polystyrene plate, 96-well format (Greiner Bio-one GmbH, Frickenhausen, Germany)
9.21 Vortex-mixer (KS-1, Janke & Kunkel)

### 10. Software

The BioPlex apparatus is operated with Bio-Plex Manager software 4.1.

### 11. Procedure

11.1 Oxidation and Desalting of LPS Solution

SAFETY PRECAUTION: Lipid polysaccharides are potent immunogens, which can bring sensitive persons into a septic shock upon intake or inhalation. Precautions should be made to prevent contact with this biochemical.

Sodium periodate is an oxidizing agent and may cause explosions when brought into contact with strong reducing agents.

The procedure utilizes sodium cyanoborohydride. The procedure should, therefore, be carried out with precautions, such as using hand gloves and a mask. Use this substance only in a chemical fume hood. The material is very toxic to aquatic organisms and may cause long-term adverse effects in the aquatic environment. The material and solution waste should be disposed, of as hazardous waste.

11.1.1 Oxidation
11.1.1.1 Add 500 μl acetate buffer, c=100 mmol/l, pH 5.5 (8.2.4) to dry LPS (8.1.9; see safety precaution).
11.1.1.2 Vortex (9.21) the solution (11.1.1.1) and sonicate (9.18) for 20 minutes and observe the reconstitution process so that all LPS is dissolved.
11.1.1.3 Add 20 μl periodate solution, c=50 mmol/l (8.2.18) to the LPS solution (11.1.1.2).
11.1.1.4 Vortex (10.21) the solution (11.1.1-3).
11.1.1.5 Incubate on ice for 40 minutes protected from light.
11.1.1.6 Quench oxidation by desalting the solution (11.1.1.4) as described in 11.1.2.

11.1.2 Desalting
11.1.2.1 Place NAP-5 column(s) (8.4.1) on manifold (9.19).
11.1.2.2 Condition the column(s) (11.1.2.1) by passing three 3-ml portions of acetate buffer, c=10 mmol/l, pH 4.0 (8.2.2) over the column bed on a flow generated by gravity only. Allow the buffer to enter the gel bed completely.
11.1.2.3 Pipette 0.5 ml oxidized LPS solution (11.1.1.5) on the column. Allow the sample to enter the gel bed completely. The flow-through is not collected.
11.1.2.4 Elute oxidized LPS with 1 ml acetate buffer, c=10 mmol/l, pH 4.0 (8.2.2). Collect eluate in a 5-ml glass tube (9.9).
11.1.2.5 Vortex (9.21) the solution (11.1.2.4) for ten seconds and add 2 μL Proclin 150 (8.1.7).
11.1.2.6 When not immediately used (11.1.2.5) store samples at 4° C. to 7° C.
11.1.2.7 Prior to immobilization, the LPS-containing solution (11.1.2.5) that can be used for different matrix and species applications is diluted as indicated in the following Tables 34 and 35.

TABLE 34
Amount of LPS solution used to immobilize beads for detection of
antibodies to Salmonella O-antigens in swine sera.

| LPS type | LPS stock solution | Added volume of acetate buffer, |
|---|---|---|
| (8.1.9) | (11.1.2.6) in μl | pH 4.0 (8.2.2) in μl |
| Se | 75 | 225 |
| Sg | 75 | 225 |
| Sl | 75 | 225 |
| Sm | 75 | 225 |
| St | 75 | 225 |

TABLE 35
Amount of LPS solution used for the immobilization to fluorescent
beads for detection of antibodies in chicken sera reacting
with Salmonella O-antigens.

| LPS type | LPS stock solution | Added volume of acetate buffer, |
|---|---|---|
| (8.1.9) | (11.1.2.6) in μl | pH 4.0 (8.2.2) in μl |
| Se | 150 | 150 |
| Sg | 150 | 150 |
| Sl | 150 | 150 |
| Sm | 150 | 150 |
| St | 150 | 150 |

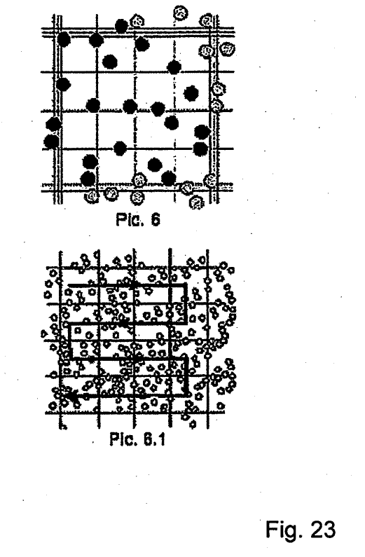

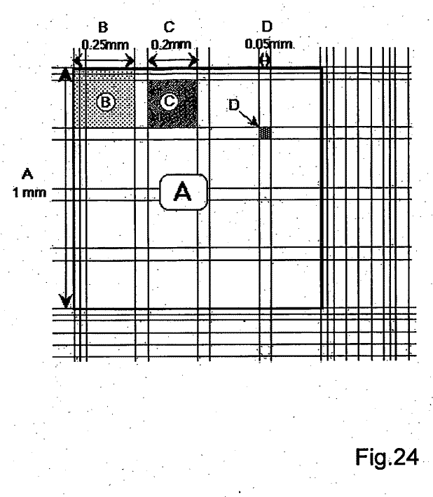

11.2 Immobilization of LPS to Beads
11.2.1 Beads (8.4.2) are vortex-mixed for minimally one minute.
11.2.2 Transfer a portion of 300 ILL beads (11.2.1) into a fresh container (9.16).
11.2.3 Centrifuge at 14,000 g for five minutes.
11.2.4 Remove supernatant carefully from the beads using a 200 μl pipette (9.6).
11.2.5 Leave a small amount of solution (10 μL) in the vial and mix the beads in the remaining solution on a vortex (9.21).
11.2.6 Thaw two portions of 100 μl EDC (8.2.7.1).
11.2.7 Thaw two portions of 100 μl NHS solution (8.2.9.1).
11.2.8 Mix 180 μl of EDC (11.2.6) and 1801 of NHS (11.2.7).
11.2.9 Transfer 300 μL EDC/NHS mix (11.2.8) to the beads (11.2.4) and suspend rigorously using a pipette.
11.2.10 Facilitate reaction on a gyro rocker (9.11) for 20 minutes.
11.2.11 Centrifuge at 14,000 g for five minutes.
11.2.12 Carefully remove supernatant from beads, leave a small volume (ca. 10 μL) on top of pellet and suspend beads using vortex mixer (9.21).
11.2.13 Add 300 μL 5 mM carbohydrazide solution (8.2.6) to the beads (11.2.12) and suspend, rigorously using a pipette.
11.2.14 Facilitate reaction on a gyro rocker (9.11) for 20 minutes.
11.2.15 Centrifuge at 14,000 g for five minutes, remove supernatant from beads, leave a small volume (ca. 10 μL) on top of pellet and suspend beads using vortex mixer (9.21).
11.2.16 Add 300 μL 1 M ethanolamine solution (8.2.8) to the beads (11.2.15) and suspend rigorously using a pipette.
11.2.17 Facilitate reaction on a gyro rocker (9.11) for 20 minutes.
11.2.18 Centrifuge at 14,000 g for five minutes, remove supernatant from beads, leave a small volume (ca. 10 μL) on top of pellet and suspend beads using vortex mixer (9.21).
11.2.19 Add 300 μL diluted oxidized LPS in sodium acetate, c=10 mmol/l, pH 4.0 (11.1.2.7) and suspend rigorously using a pipette.
11.2.20 Allow reaction on a gyro rocker (9.11) for 90 minutes.
11.2.21 Centrifuge at 14,000 g for five minutes, remove supernatant from beads, leave a small volume (ca. 10 μL) on top of pellet and suspend beads using vortex mixer (9.21).
11.2.22 Add 300 μL cyanoborohydride solution, c=100 mmol/l (8.2.14) and suspend rigorously using a pipette.
11.2.23 Facilitate reaction on a gyro rocker (9.11) for 60 minutes.
11.2.24 Centrifuge at 14,000 g for five minutes and carefully remove supernatant from beads, leave a small volume (ca. 10 μL) on top of pellet and suspend beads using vortex mixer (9.19).
11.2.25 Add 300 μL PBS (8.2.11).
11.2.26 Add 1 μL Proclin 150 (9.1.7) and mix suspension.
11.2.27 The beads are ready for testing Salmonella antibodies in biological materials.
   1.1.2.28 Counting of LPS coupled beads.
   11.2.28.1 Vortex LPS coupled beads suspensions (11.2.25) and transfer 1 μL in a fresh 1-mL tube (9.15).
   11.2.28.2 Dilute by adding 24 μL PBS, c=10 mmol/l, pH 7.2 (8.2.11) and mix.
   11.2.28.3 The external supports of a Bürker-Turk (9.3) counting chamber are to be moistened with Milli-Q® water (8.1.17) and the cover glass (9.13) is gently pushed onto the counting chamber from the front.
   11.2.28.4 Fill a pipette with 20 μl bead solution (11.2.28.2), gently form a drop at the tip of the pipette.
   11.2.28.5 This drop (11.2.28.4) is to be placed between the cover glass and the counting chamber.
   11.2.28.6 As a result of the capillary effect, the gap between the cover glass and the chamber base fills up. Before the bead solution can overflow at the edges of the chamber section, the tip of the pipette-must be removed. If any air bubbles are visible or if the liquid has overflowed over the edges and into the grooves, the chamber must be cleaned and feeding must be repeated.
   1.1.2.28.7 Place the filled counting chamber under a microscope (9.12) and magnify the image with a 10× object.
   11.2.28.8 The count should be started at the top left-hand corner and follow the direction shown by the arrow (FIG. 23, lower panel). Counting may be enhanced with the microscopes illumination reduced.
   11.2.28.9 Count the number of beads in 16 squares (FIG. 23, upper panel) inside the thick lined area (see FIG. 24).
   11.2.28.10 Notes on counting:
      11.2.28.10.1 Use reduced microscope illumination for all chambers.
      11.2.28.10.2 The difference of the counter cells in the large squares and the group squares must not exceed ten cells.
      11.2.28.10.3 Double checks must be performed for all cell counts. After counting the two counting nets, the bottom counting net is to be counted in the same way as a check. When doing this, it is to be ensured that the chamber has not dried out. This can be prevented by filling the bottom chamber only shortly before the count and the counting after the sedimentation time.
      11.2.28.10.4 The difference between the totals of the counts for the two counting nets must not exceed ten cells. The average value of the counts is then used in the calculation formula or multiplied by the corresponding factor.
   11.2.28.11 Multiply the counted number (11.2.28.9) with the dilution factor (25×) divided by counted area (1 mm²) multiplied with chamber depth to calculate the concentration of beads per ml.

11.3 Detection of Anti-Salmonella Antibodies

Note: Ensure that all solvent and reagent reservoirs contain sufficient volume for complete method run before initiating the assay.
11.3.1 Make the BioPlex apparatus (9.2) operational by a 30-minute laser warming up step, followed by a start up and calibration procedure with appropriate calibration solvents (8.1.3) according to the quick guide.
1.3.2 Mix and dilute LPS-coated beads (11.2.26) with PBS, c=10 mmol/l, pH 7.2 (8.2.11) so that the concentration of each bead equals 5000 per ml.
11.3.3 Transfer 50 μL bead mix (11.3.2) into a microtiter plate already filled with diluted monoclonal anti-O-antigens (8.3.7).
11.3.4 Incubate for 30 minutes on a microtiter plate shaker (9.14).
11.3.5 Add 10 μL five times diluted anti-mouse Ig-PE (8.2.12).
11.3.6 Incubate for 15 minutes on a microtiter plate shaker (9.14).
11.3.7 Note in logging sample wells and their contents.
11.3.8 Place plate in BioPlex (9.2), set maximal counting time to 120 seconds and count at least 50 beads per LPS group.
11.3.9 Activate software program to count fluorescence of beads, which had captured (fluorescent) antibodies.

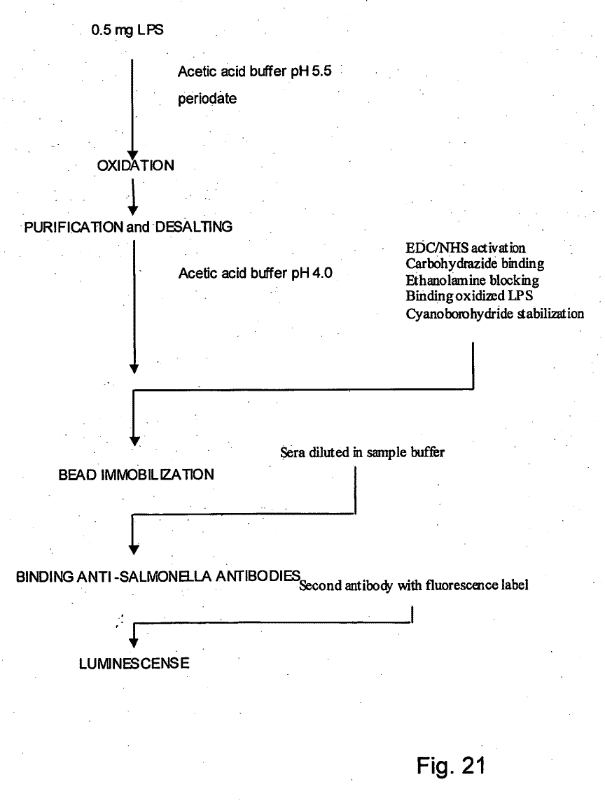

For a schematic representation of the procedure, see FIG. 21.

Typical responses of Salmonella monoclonal antibodies are presented in Table 36.

TABLE 36
Typical responses of reference sera incubated with LPS-coated beads
and a secondary fluorescent antibody providing the signal.

| | LPS B | LPS C₁ | LPS C₂ | LPS D |
|---|---|---|---|---|
| anti-O4 | 305 | 76 | 95 | 88 |
| anti-O5 | 6398 | 78 | 91 | 79 |
| anti-O7 | 86 | 174 | 91 | 98 |
| anti-O8 | 90 | 86 | 1668 | 91 |
| anti-O9 | 82 | 81 | 82 | 145 |

### Example 10

### 1. Mild Periodate Oxidation

The success of the final binding of anti-Salmonella antibodies, and thus the screening of invasive infections in the animal, is much dependent of the oxidation of the monosaccharide constituents of the polysaccharide part of LPS, and the oligosaccharide part of the core region of LPS. It can be deduced from the described structures for the different serotypes of, e.g., Salmonella, that oxidation may lead to a breakdown of the antigenic structures, which are, in particular, part of the polysaccharide part of LPS.

Whereas the oxidation of hexitols occurs rapidly, parynosides, which are predominantly occurring in Salmonella LPS, need a higher periodate concentration to facilitate the oxidation in the same time. Pyranosides, which possess α-erythro-hydroxyl groups, such as in arabino, galacto or manno configurations like in Salmonella spp. LPS, are more easily oxidized than α-threo-hydroxyl groups, such as in xylo or gluco variants. It should be realized that while the ring is opened and aldehyde functions for attachment of, e.g., protein, molecules are created, α-hydroxy carbonyl compounds also may be created, which may oxidize again if periodate is still present. It is, therefore, that non-conjugated monosaccharides, which are thus not part of an oligo- or polysaccharide, are completely destroyed by a periodate oxidation to formic acid and formaldehyde at sufficient high concentrations of the oxidizer. At relatively high concentrations of periodate, 1,3-diketones and also di-axial diols can be oxidized.

A breakdown or oxidation reaction, more than only the creation of aldehyde groups, would, therefore, lead to a failure to detect an infection in a sample of biological material, despite a good coupling reaction to a solid phase supported by the presence of a polyamine-containing molecule, such as a protein. In other words, a mild periodate reaction is needed to leave the antigenic structure intact, but just enough to enable a coupling between protein and thus solid phase.

### 2. Result

LPS from Se, Sg, Sl and St was oxidized for 40 minutes at pH 5.5 using a range of sodium m-periodate concentrations. Following oxidation, LPS was coupled to a biosensor surface and immobilization efficiency and antigenic activities was monitored.

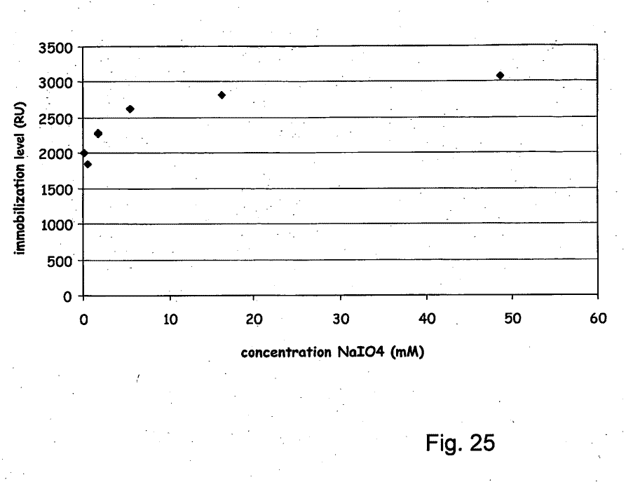

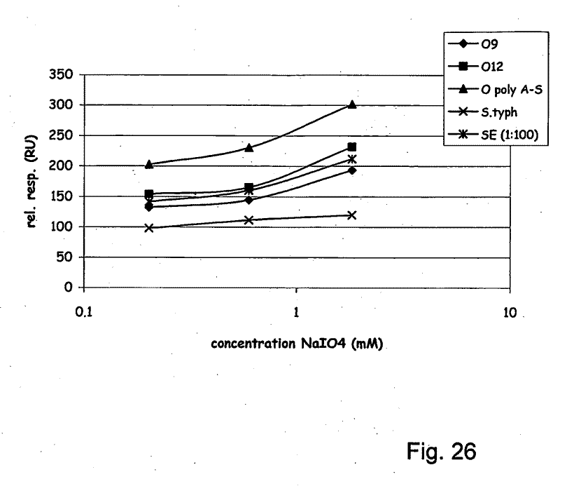

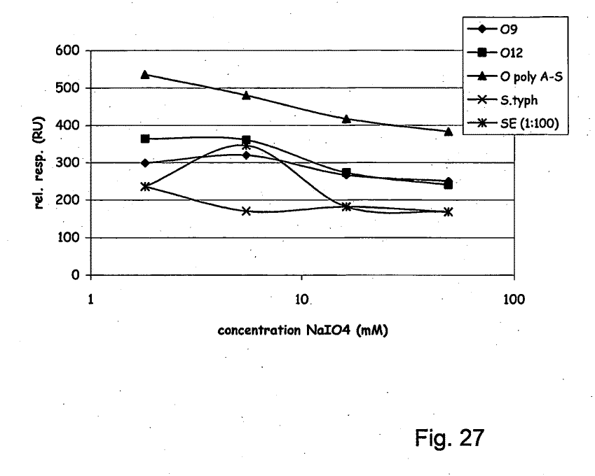

From FIG. 25, it is obvious that a higher oxidation grade of LPS from S. enteritidis gave rise to a corresponding higher coating level at the biosensor chip. However, despite the higher immobilization levels, the response from the antibody probing decreases as demonstrated in FIGS. 26 and 27. For this LPS type, an optimum periodate concentration of 1.8 mM was determined.

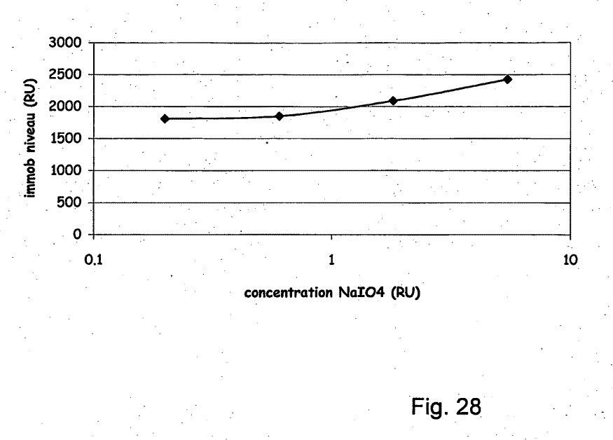

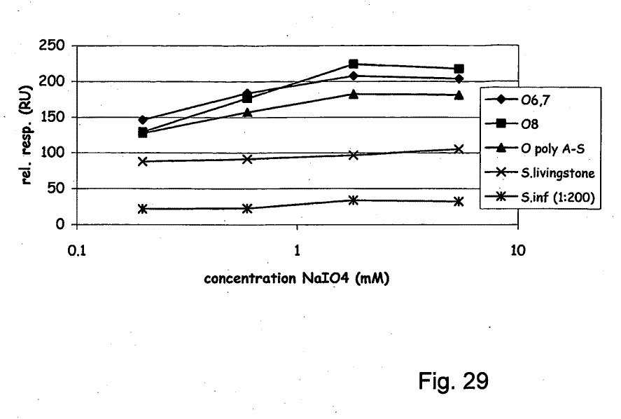

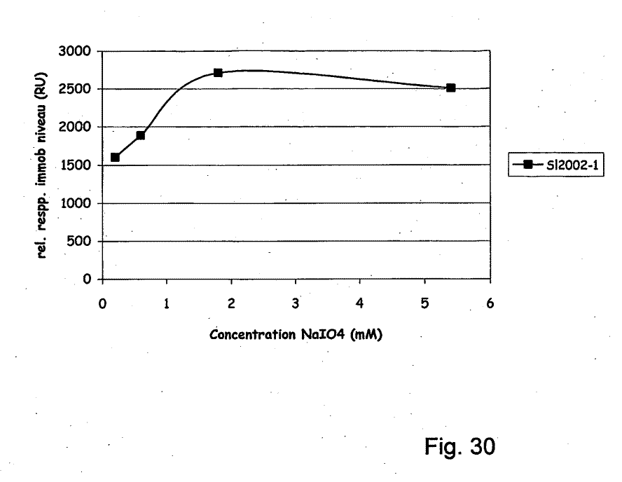

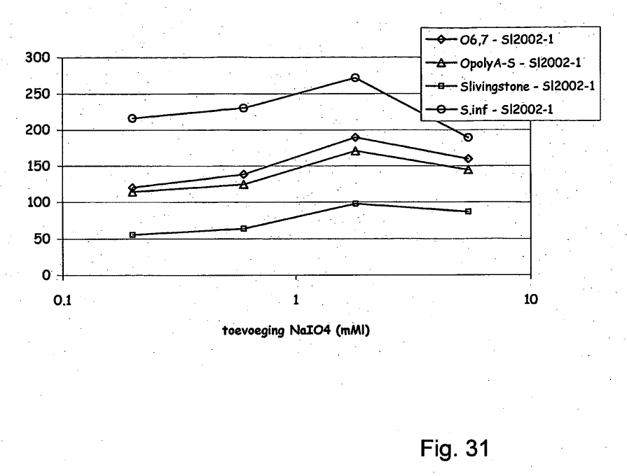

In a similar way, the effects of oxidation on the immobilization and antigenic activity of LPS from S. goldcoast were tested (FIGS. 28 and 29). An identical optimum for the sodium periodate concentration was found at 1.8 mM. Similar results were obtained for S. livingstone (FIGS. 30 and 31). An optimum of 1.8 mM m-periodate was found here as well.

### Example 11

### Extraction of Lipopolysaccharides from Salmonella spp.

WARNING AND SAFETY PRECAUTIONS: Salmonella spp., which are used in this extraction method to obtain lipopolysaccharides (LPS), belong to risk class 2 microorganisms. Risk class 2 microorganisms are described as follows: they can induce illnesses like diarrhea and fever, but spreading of the disease is improbable and there are effective prophylaxes or treatment possible. When working with live bacteria, the procedure should, therefore, be carried out with precautions like disinfecting equipment and hands with 70% ethanol. The waste of: living Salmonella spp. should be destroyed or disposed of by autoclavation or as biohazard waste, respectively.

LPS compounds are highly pyrogenic and can cause fever. To avoid intake, treat aqueous solutions of LPS with care and wear a mask when working with solid material. If any of these compounds enters the bloodstream, immediately seek medical attention.

### 1. Introduction

Salmonella is a gram-negative bacterium, and its outer membrane consists of various antigenic structures, including flagella, outer membrane proteins and Lipopolysaccharides (LPS). The molecule of LPS consists of a so-called lipid A part, which is embedded in the leaflet of the outer membrane, a core region and polysaccharide. The core region is composed of two or three heptoses and two or three residues of eight-carbon, negatively charged monosaccharides KDO. The core region links lipid A to the polysaccharides, which is also known as the O-side chain. This O-side chain is highly variable with respect to its length and composition between strains, but also within a strain influenced by growth conditions of the Salmonella. Despite variation, antigenic structures coded in the PS are unique for a certain Salmonella serovar. In fact, antigenic structures O3, O4, O6/7, O8, O9, O10 and O12 represent approximately 90% of known Salmonella serovars occurring on porcine products, in particular, Dutch abattoirs.

To detect a humoral response to O-antigens as an indication of an exposure of farm animals to Salmonella, LPS can be used to probe the binding of raised antibodies to these biomolecules. For this purpose, LPS from S. typhimurium (O4, O5, O12), S. enteritidis (O9, O12), S. livingstone (O6/O7), S. goldcoast (O6, O8) and S. meleagridis (O3, O10) can be extracted.

An in-house extraction is paramount because LPS from only a limited number of Salmonella serovars is commercially available. Furthermore, in-house production can secure a continuous availability of LPS types for a successful antibody detection assay. The in-house extraction method described here for this purpose is based on a protocol described by Staub (0). Trichloroacetic acid (TCA) extracts LPS containing 1% to 10% protein contamination. This product is suitable for covalent immobilization of LPS to a carboxymethylated dextran layer coated on a gold metal surface of a biosensor chip (see SOP CHEMIE/A22 (Example 12)). This chip immobilized with LPS, in combination with a Biacore optical SPR biosensor system, can be used to trace Salmonella-LPS antibodies in sera, also known as serology.

### 2. Scope and Field of Application

This method describes the extraction of LPS from several Salmonella serovars with the use of trichloric acetic acid. Extracted LPS is suitable for modifications to facilitate its immobilization on a carboxymethylated dextran surface.

### 3. References

Staub, A. M., Methods in Carbohydrate Chemistry, 5, 92 (1965)

SOP Chemie/A22: Immobilization of Salmonella-derived LPS onto a biosensor chip (Biacore) and detection of serum antibodies reporting a current or past Salmonella infection (Example 12).

SOP Chemie/A23: Optimalization protein addition to LPS for immobilization and detection of serum antibodies (Example 13).

### 4. Definitions

c=concentration in % (m/v), % (v/v), mol/l or mmol/l as indicated.

### 5. List of Abbreviations

5.1 BGA: Brilliant Green agar

5.2 BHI: Brain Heart Infusion

5.3 BHIa: Brain Heart Infusion agar

5.4 LPS: Lipopolysaccharides

5.5 NB: Nutrient broth

5.6 NaCl: Sodium chloride

5.7 NaOH: Sodium hydroxide

5.8 TCA: Trichloric acetic acid

### 6. Principle

Lipopolysaccharides (LPS) are produced by the extraction of Salmonella with the aid of trichloricacetic acid (TCA). Salmonella is cultured on and then collected from Brain Heart Infusion agar plates. After several washing steps with a saline solution and several centrifugation steps, TCA is added. The acidified suspension is incubated at a low temperature for three hours to solubilize LPS from bacterial cells. The suspension is centrifuged to remove cellular material and the pH is neutralized. LPS is then partly purified and concentrated by ethanol precipitation at low temperature. Finally, salts and ethanol are removed by dialysis, and remaining particles in the retained LPS-containing solution are spun down by centrifugation. The supernatant is lyophilized and weighed to determine the recovery of produced LPS.

### 7. Reagents and Materials

During the procedure, unless stated otherwise, use only reagents of recognized analytical grade and only distilled water or water of equivalent purity. Reference to a company is for information and identification only and does not imply a recommendation unless so stated.

7.1 Chemicals
7.1.1 Brilliant Green Agar (Oxoid, Basingstroke, England, CM329)
7.1.2 Brain Heart Infusion (Oxoid, CM225)
7.1.3 Brain Heart-Infusion Agar (Oxoid, CM375)
7.1.4 Ethanol, absolute (Merck, Darmstad, Germany, 1.00983.2500)
7.1.5 Glycerol 87% (Merck, 1.04091.1000)
7.1.6 Nutrient Broth No. 2 (Oxoid, 67)
7.1.7 Sodium chloride (Merck, 1.06404.1000)
7.1.8 Sodium hydroxide (Merck, 1.06498.1000)
7.1.9 Ethyleen glycol (Merck, 9621.2500))
7.1.10 Trichloroacetic acid (Merck, 1.00807.0250)
7.1.11 water was obtained from the MILLI-Q® purification system (9.24)

7.2 Salmonella Agglutination Sera
7.2.1 anti-O4 (Pro-Lab Diagnostics, Salmonella reference section of the Central Veterinary Laboratory, Weybridge, Great Britain)
7.2.2 anti-O5 (Pro-Lab Diagnostics)
7.2.3 anti-O6,7 (Pro-Lab Diagnostics)
7.2.4 anti-O8 (Pro-Lab Diagnostics)
7.2.5 anti-O9 (Pro-Lab Diagnostics)
7.2.6 anti-O12 (Pro-Lab Diagnostics)
7.2.7 anti-O-Poly A-S (antisera to groups A through S) (Pro-Lab Diagnostics)
7.2.8 anti-O-Poly E (antisera to factors O3, O10, O15, O19, O34) (Pro-Lab Diagnostics)

7.3 Bacterial Strains
7.3.1 Salmonella enteritidis (#23, phage type 1 strain RIVM, The Netherlands; 90-16-706)
7.3.2 Salmonella goldcoast (Division's working bank, Utrecht University, The Netherlands)
7.3.3 Salmonella livingstone (Division's working bank)
7.3.4 Salmonella meleagridis (Division's working bank)
7.3.5 Salmonella typhimurium X-193 (ASG, Lelystad)

7.4 Reagents

Unless stated otherwise, all prepared media can be stored for three months at 2° C. to 8° C.

After dispensation of agar media in petri dishes, the plates are cooled at room temperature. After hardening of agar, the plates are inverted and dried at room temperature for two to three days on the tabletop.
7.4.1 Brilliant Green agar (BGA) plates: Suspend 52 g of BGA (7.1.1) in 1.0 l water (7.1.11). Boil to dissolve the medium completely. Mix well and dispend 15 ml portions in petri dishes (8.29).
7.4.2 Brain Heart Infusion (BHI) broth: Dissolve 37 g of BHI broth (7.1.2) in 1.0 l water (7.1.11). Mix well, distribute into final containers and sterilize by autoclaving at 121° C. for 15 minutes.
7.4.3 Brain Heart Infusion Agar (BHIa): Suspend 47 g BHI agar (7.1.3) in 1.0 l water (7.1.11). Boil to dissolve the medium completely. Mix well and dispend 15 ml portions in petri dishes (8.29).
7.4.4 Cooling solution: Mix 1.0 l ethylene glycol (7.1.9) with 3 l water
7.4.5 Cold ethanol: Store 1 l ethanol (7.1.4) o/n in a freezer (−18° C.) (8.17).
7.4.6 Glycerol 87% sterile: Autoclave 50 ml glycerol (7.1.5) at 121° C. for 15 minutes.
7.4.7 Nutrient broth (NB): Dissolve 25 g NB (7.1.6) in 1.0 l water (7.1.11). Mix well, distribute into 100 ml flasks and sterilize by autoclaving at 121° C. for 15 minutes.
7.4.8 Saline (c=−0.9% (m/v)): Dissolve 9 g sodium chloride (NaCl) (7.1.7) in 1.0 l water: (7.1.11). Before use, cool the saline overnight in a refrigerator.
7.4.9 Trichloroacetic acid (TCA) solution, c=0.25 mol/l: Dissolve 40.8 g TCA (7.1.10) in 1.0 l water (7.1.11).
7.4.10 Trichloroacetic acid (TCA) solution, c=0.50 mol/l: Dissolve 81.7 g TCA (7.1.10) in 1.0 l water (7.1.11).
7.4.11 Sodium hydroxide (NaOH) solution, c=5.0 mol/l: Dissolve 40 g NaOH (7.1.8) in 200 ml water (7.1.11).
7.4.12 Sodium hydroxide (NaOH) solution, c=0.10 mol/l: Dilute 1.0 ml, c=5 mol/l NaOH (7.4.1.1) in 49.0 ml water (7.1.11).

### 8. Equipment and Apparatus

Reference to a company is for information and identification only and does not imply a recommendation unless so stated. Equivalent equipment and apparatuses may be appropriate as well.
8.1 Analytical balance (type AE240, Mettler, Zürich, Switzerland), weighing range 0 to 40 g (SOP CHEMIE/011)
8.2 Balance (type MC1 LC2000P, Sartorius Instrumenten. BV, Nieuwegein, The Netherlands)
8.3 High speed centrifuge (T-124 Centrikon, Kontron, Zurich, Switzerland)
8.4 Superspeed centrifuge (RC-5B, Sorvall, Newton, Conn., USA)
8.5 Centrifugal polypropylene tubes, for Centrikon 290 ml (253483, Beun de Ronde, Abcoude, The Netherlands)
8.6 Centrifugal tubes for rotor SM-24, Sorvall (Sorvall)
8.7 Conical tubes (50 ml) (Cellstar 210261, Greiner Bio-One, Alphen a/d Rijn, The Netherlands)
8.8 Conical tubes (15 ml) (Cellstar 188271, Greiner Bio-One)
8.9 Cryogenic tubes (1.8 ml) (Cryo's 122261, Greiner Bio-One)
8.10 Dialysis Membrane clamps (Part#CB-1050, Cellu Sep, Membrane Filtration Products Inc., Seguin, Tex., USA)
8.11 Spectra/Por dialysis tubing MWCO 1000 (Spectrum Laboratories Inc., Rancho Dominguez, Calif., USA)
8.12 Finn pipette, 200 to 1000 μl. (Labsystems Oy, Helsinki, Finland)
8.13 Finn pipette, 40 to 200 μl (Labsystems Oy, Helsinki, Finland)
8.14 Finn pipette, 5 to 40 μl (Labsystems Oy, Helsinki, Finland)
8.15 Freeze dry apparatus (7570001, Labconco Corporation, Kansas City, Mo., USA)
8.16 Freezer −18° C., freeze range between −16° C. to −25° C. (Elbanton, Kerkdriel, The Netherlands)
8.17 Freezer −80° C. (U57085 Classic, New Brunschwick Scientific BV, Nijmegen, The Netherlands)
8.18 Glass beaker, 15 l (Schott-Duran, Omnilabo International BV, Breda, The Netherlands)
8.19 Glass slides (AA00000102E, Menzel-Glaser, Braunschweig, Germany)
8.20 Ice salt bath: mix approximately 150 g NaCl (8.1.7) with 3 l ice crunch after addition of 150 ml of tap water
8.21 Home made −4° C. incubator (measured temperature of contact liquid to ethanol precipitating LPS extract (10.3.37), approximately 1° C.)
   8.21.1 Circulation chiller; set temperature −4° C. (WK230, Lauda, Lauda-Königshofen, Germany)
   8.21.2 Central cooling core PROTEAN II (165-1806, BioRad Laboratories BV, Veenendaal, The Netherlands)
   8.21.3 Polystyrene box & fitting plastic inner bowl
8.21.3.1 Whole system (9.22.1, 9.22.2) was filled with cooling solution (8.4.4)
8.22 Magnetic stirrer (EM3300T, LaboTech B.V., Ochten, The Netherlands)
8.23 MILLI-Q® water purification system (Millipore, Bedford, Ma, USA)
8.24 pH meter (type pH 537, WTW. Weilheim, Germany)
8.25 Refrigerator (Elbanton)
8.26 Rotor Centrikon (A6.14, Kontron)
8.27 Rotor Sorvall (SM-24, Sorvall)
8.28 Spatula, Drigalski glass (408014, Omnilabo International BV)
8.29 Petri dishes (633102, Greiner Bio-One)
8.30 Powerpette plus (Jencons, Bedfordshire, England)
8.31 Vortex-mixer (KS-1, Janke & Kunkel, Staufen, Germany)

### 9. Procedure

9.1 Preparation of stock culture
9.1.1 Make an isolate of Salmonella (7.3) by spreading one colony, or the content of an inoculation loop onto a BGA plate (7.4.1).
9.1.2 Incubate the plate (9.1.1) overnight at 37° C.
9.1.3. A single colony is picked from the plate (9.1.2) with an inoculation loop and suspended in 100 ml NB (7.4.7).
9.1.4 Incubate overnight at 37° C.
9.1.5 Add 50 ml glycerol (7.4.5) to 100 ml cultured NB medium (9.1.4).
9.1.6 Aliquot culture/glycerol mixture (9.1.5) into eleven 14 ml (8.9) (volume of 12.5 ml) and twelve 1.5 ml (8.10) (volume of 1 ml) sterile tubes.
9.1.7 One of the 1.5 ml tubes is marked as standard bank. The contents of this tube will oily be used to prepare more aliquots of 1 and 12.5 ml via the method here described (9.1).
9.1.8 Quickly freeze the aliquots at −80° C. (9.1.6) and store until use.

9.2 Determination of Salmonella Isolate Purity by Agglutination
9.2.1 Pipette 25 μl (8.15) sterile saline (7.4.8) onto a glass slide (8.20).
9.2.2 Suspend one colony from the plate-cultured Salmonella (9.1.2) in the saline (7.4.8).
9.2.3 Add a single drop of agglutination serum (7.2) using the facilitated container drop system.
9.2.4 Mix sera and suspension by gently tilting the slide back and forth for two minutes.
9.2.5 Determine agglutination by looking for aggregation formation in front of a black background.
9.2.6 Disclose the identity of Salmonella strain by comparing the results of aggregation with Table 37.
9.2.7 When the identity of the cultured strain differs from predicted aggregation in Table 37, it can be concluded that the tested culture was not (exclusively) composed of the expected Salmonella serotype. In such case, a new stock culture has to be prepared.

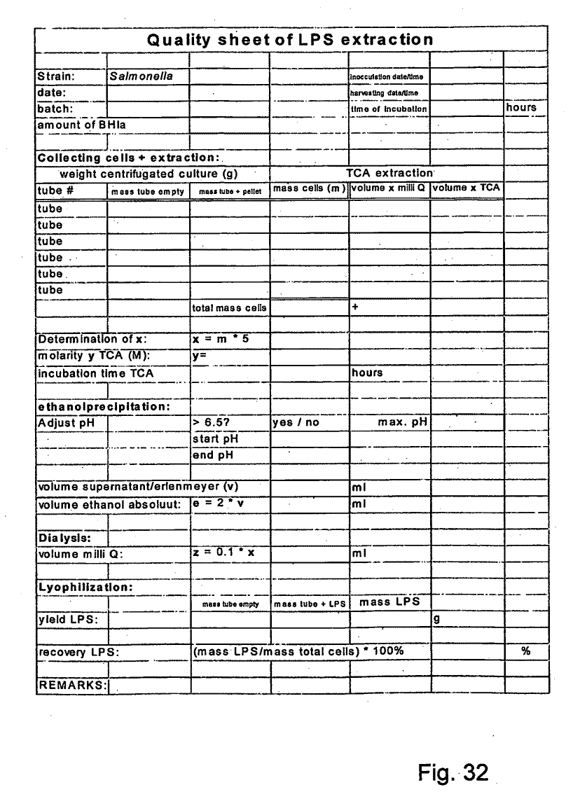

9.3 Extraction Method
9.3.1 Prepare 120 BHI agar plates (7.4.3).
9.3.2 Thaw a 12.5 ml tube with Salmonella stock culture (9.1.8).
9.3.3. Spread 100 μl (8.14) stock culture (9.1.8) on each plate (9.3.1) with a spatula (8.29).
9.3.4 Incubate overnight at 37° C.
9.3.5 Fill a 15 l container (8.19) with 10 l water (7.1.11), and cool to 4° C. to 8° C. in a refrigerator (8.26) until further use.
9.3.6 Weigh (8.3) six empty centrifugation tubes (8.6) and note their weight on the quality sheet (see FIG. 32).
9.3.7 Put Milli-Q® (7.1.11) and TCA (7.4.9, 7.4.10) containers in prepared ice salt bath (8.21).
9.3.8. Take twenty plates (9.3.4) overnight incubated plates and add 1 ml of saline to each plate.
9.3.9 Harvest the bacteria by lightly scraping (making round movements) a Drigalski spatula (8.29) over the agar, making a suspension of the bacteria in saline.
9.3.10 Collect the suspension in one of the six pre-weighed centrifugation tubes (9.3.6).
9.3.11 Wash each plate (9.3.9) twice with 2 ml saline (7.4.8) solution.
9.3.12 Combine the suspensions (9.3.11) with the contents of the centrifugation tube (9.3.10).
9.3.13 Repeat steps 9.3.8 through 9.3.12 five times for the remaining plates (9.3.4).
9.3.14 Add 100 ml saline (7.4.8) to each centrifugation tube.
9.3.15 Tare the tubes (9.3.14).
9.3.16 Centrifuge for 15 minutes at 10,000×g and 4° C. (8.4, 8.27).
9.3.17 Decant the supernatant in a waste container.
9.3.18 Suspend each bacterial pellet in 10 ml saline (7.4.8) until a smooth suspension is formed.
9.3.19 Add 75 ml saline (7.4.8) to each centrifuge tube.
9.3.20 Repeat steps 9.3.14 to 9.3.19 once.
9.3.21 Repeat steps 9.3.15 to 9.3.17 once.
9.3.22 Weigh (8.3) the tubes with bacterial pellet (9.3.21) and note weigh results on the quality sheet (see FIG. 32).
9.3.23 Determine the mass (=m) of the deposited bacteria by subtracting the weight of the empty (9.3.6) with that of the cell-containing tubes (9.3.22).
9.3.24 Suspend the bacterial pellets (9.3.22) with x ml (for determination x, see Table 38) of water (7.1.11) by repeatedly drawing in and washing with a 10 ml pipette (8.31).
9.3.25 Combine the suspensions of two centrifugation tubes in one tube.
9.3.26 Repeat 9.3.25 for the remaining centrifugation tubes.
9.3.27 Add x ml of y M TCA (7.4.10) (see Table 38 for values x and y).
9.3.28 Insert stirring rods in each of the suspensions (9.3.27).
9.3.29 Stir the suspensions (9.3.28) for three hours at 4° C. on a magnetic stirrer (8.23).
9.3.30 Remove the stirring rods.
9.3.31 Centrifuge the suspensions for 30 minutes at 20,000×g and 4° C. (8.5, 8.28).
9.3.32 Collect the supernatants of the centrifugation tubes in a 500-ml beaker.
9.3.33 Adjust the pH of the supernatant with 5 M NaOH (7.4.11) and 0.10 M NaOH (7.4.12) to pH 6.5.
9.3.34 Determine the volume (v) of the pH adjusted supernatant (9.3.33).
9.3.35. Cool the supernatant to freezing point by putting the filled flasks (9.3.34) in a −18° C. freezer (8.17) for 30 minutes.
9.3.36 Add 2* e ml (for values of e, see Table 38) freeze-cold ethanol (7.4.5).
9.3.37 Cool the solution overnight at −4° C. (8.21).
9.3.38 Dispense the solution in six centrifuge tubes (8.6).
9.3.39 Centrifuge the solution for 30 minutes at 20,000×g and −4° C. (8.4, 8.27).
9.3.40 Cut three parts (each 1.0 cm of length) from the dialysis tube (8.12).
9.3.41 Wash the dialysis tubes (9.3.40) briefly with water (7.1.11) and keep them wet in water (7.1.11) until further use.
9.3.42 Clip a membrane clamp (8.11) at one end of the dialysis tube (9.3.41), leaving 1 cm tubing free.
9.3.43 Decant the supernatant (9.3.39) in a waste bottle.
9.3.44 Remove the remaining supernatant from the centrifuge tubes with the help of a pipette (8.13).
9.3.45 Add one ml of water (7.1.11) to each centrifuge tube (9.3.44).
9.3.46 Suspend the pellets by drawing in and washing out with a one-ml pipette (8.13).
9.3.47 Fill one of the prepared dialysis bags (9.3.42) with the re-suspended pellets (9.3.46) of two centrifuge tubes.
9.3.48 Wash each of the tubes with (z−1)/6 ml (for the value of z, see Table 38) water (7.1.11) and combine the wash with the contents of the dialysis bag (9.3.47).
9.3.49 Clip another clamp (7.10) on top of the filled dialysis tube (9.3.48) leaving a small air bubble between solution and clamp.
9.3.50 Repeat the steps 9.3.42 to 9.3.49 for the remaining centrifuge tubes.
9.3.51 Place the three filled dialysis tubes in pre-cooled water (9.3:5) at 4° C.
9.3.52 Incubate by dialyzing the contents of the tubes (9.3.51) for two days under continuous gentle stirring conditions at 4° C. on a magnetic stirrer (8.23).
9.3.53 Refresh the dialysate at least twice, by exchanging the water with 7 l fresh, precooled water (7.1.11).
9.3.54 Collect the contents of the dialysis tubes in a 50-ml container (8.8).
9.3.55 Divide the collected volume (9.3.54) in centrifuge tubes (8.7).
9.3.56 Centrifuge the tubes for 20,000×g for 30 minutes at 4° C. (8.5).
9.3.57 Weigh an empty 50-ml container (8.8) on an analytic balance (8.2) (without cap) and note its weight on the quality sheet (see FIG. 32).
9.3.58 Collect the supernatant (9.3.56) in pre-weighed container (9.3.57).
9.3.59 Freeze the supernatants in an −80° C. freezer (7.15).
9.3.60 Lyophilize (7.15) the frozen supernatants (9.3.59) until a dry white crystal structure is observed.

NOTE: Handle lyophilized, LPS product carefully, as its electrostatic characteristics may easily make it air-borne.

NOTE: LPS is a well-known toxin, which when inhaled or swallowed may induce clinical effects in humans. Safety measures, such as masks, should be strictly followed to prevent any intoxication.
9.3.61 Weigh lyophilized LPS-holding container (9.3.60) on an analytical balance (8.2) and note the resulting mass on the quality sheet (see FIG. 32).
8.3.62 Calculate the yield of LPS: (weight LS (9.3.61)−weight container (9.3.57))/total mass wet cells (9.3.23)*100%.
8.3.63 Store lyophilized LPS powder in a closed container at 4° C. until further use.

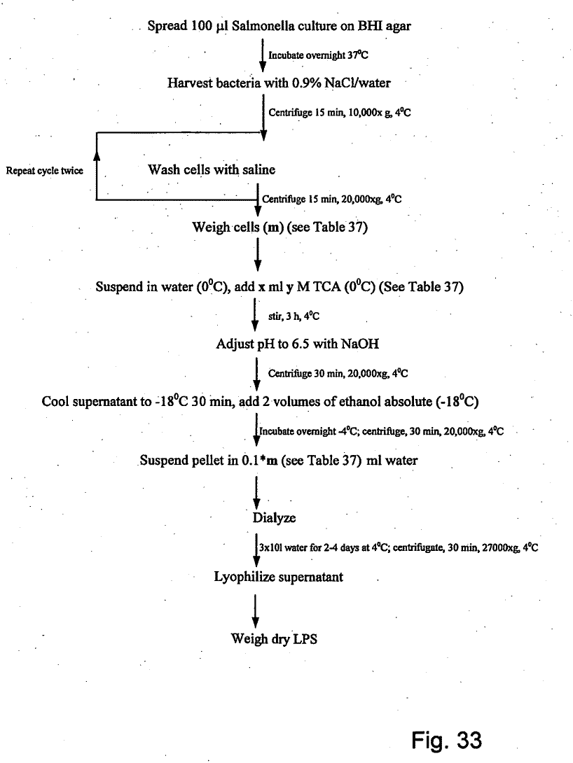

An overview of the procedure is depicted in FIG. 33.

TABLE 37
Check board for agglutination readings to disclose the identity of the
present bacteria in terms of Salmonella species and Salmonella serovar.

| | Agglutination sera | | | | | | | |
|---|---|---|---|---|---|---|---|---|
| | O4 | O5 | O6, 7 | O8 | O9 | O12 | poly A-S | poly E |
| Salmonella strain | (8.2.1) | (8.2.2) | (8.2.3) | (8.2.4) | (8.2.5) | (8.2.6) | (8.2.7) | (8.2.8) |
| S. enteritidis (7.3.1) | − | − | − | − | + | + | + | − |
| S. goldcoast (7.3.2) | − | − | + | + | − | − | + | − |
| S. livingstone (7.3.3) | − | − | + | − | − | − | + | − |
| S. meleagridis (7.3.4) | − | − | − | − | − | − | + | + |
| S. typhimurium (8.3.4) | + | + | − | − | − | + | + | − |

Legend:
+ = Aggregation formation, agglutination positive
− = No aggregation formation, agglutination negative

TABLE 38
Conversion table to determine volumes of water (x), TCA (x),
ethanol (e) and TCA concentration (y) for extraction and
precipitation, and volume of water (z) for dialysis procedures
to isolate and purify LPS from Salmonella. Letters
noted: m refers to mass (10.3.23), whereas v refers to pH
adjusted supernatant volume (10.3.34).

| | Extraction | | | |
|---|---|---|---|---|
| | x ml water | | | |
| | (7.1.11) | | | Dialysis |
| | and x ml | | Precipitation | z ml |
| | TCA | | e ml ethanol | water |
| Strain | (7.4.9, 7.4.10) | y M TCA | (7.4.5) | (7.1.11) |
| S. enteritidis | x = m * 5 | 0.25 (7.4.9) | e = 2 * v | z = |
| S. goldcoast | | 0.5 (7.4.10) | | x * 0.1 |
| S. Livingstone | | 0.5 (7.4.10) | | |
| S. Meleagridis | | n.e.y. | | |
| S. typhimurium | | 0.5 (7.4.10) | | |

n.e.y.: not established yet.

### Example 12

### Immobilization of Salmonella-Derived LPS Onto a Biosensor Chip (Biacore) and Detection of Serum Antibodies Reporting a Current or Past Salmonella Infection

WARNING AND SAFETY PRECAUTIONS: Lipid polysaccharides are potent immunogens, which can bring sensitive persons into a septic shock upon intake or inhalation. Precautions should be made to prevent contact with this biochemical.

Sodium periodate is an oxidizing agent and may cause explosions when brought in contact with strong reducing agents.

The procedure utilizes sodium cyanoborohydride. The procedure should, therefore, be carried out with precautions by using, for example, hand gloves and a mask. Use this chemical substance only in a chemical fume hood. The material is very toxic to aquatic organisms and may cause long-term adverse effects in the aquatic environment. The material and solution waste should be disposed of as hazardous waste.

### 1. Introduction

Among the components of the antigenic structure of the genus Salmonella, the somatic antigens are important as an instrument to trace immune response in animals upon an invasive infection of this organism. Somatic antigens are located on the polysaccharide part of lipid polysaccharide (LPS), which is a constituent of the bacterial cell wall. After extraction and isolation of carefully chosen LPS (see SOP CHEMIE/A21 (Example 11)), antigen-containing LPS is coupled covalently to a biosensor chip surface to serologically monitor samples for the presence of anti-Salmonella antibodies through their binding to the immobilized antigen-containing LPS on the chip surface (see SOP CHEMIE/A23 (Example 13)). This SOP describes the method for oxidation, immobilization of LPS onto the biosensor chip (BIACORE) and the analysis of antibodies in sera.

### 2. Scope and Field of Application

To analyze serum samples from chicken for the presence of anti-Salmonella antibodies reacting with O4, O5, O6, O7, O8, O9 and O12 somatic antigens.

### 3. References

Concentration Analysis Handbook, Version AA, December 2001, Biacore AB, Uppsala, Sweden.

BIAapplications Handbook, version AB (reprinted 1998), Biacore http://www.jp.amershambiosciences.com/tech_support/manual/pdf/dnapuri/52207400afpdf.

SOP Chemie/A21: Extraction of lipopolysaccharides from Salmonella spp. (Example 11).

SOP Chemie/A23: Optimalization of protein addition to LS for immobilization and detection of serum antibodies (Example 13).

### 4. Definitions

c=concentration in % (m/v), % (v/v), mol/l or mmol/l as indicated.

### 5. List of Abbreviations

5.1 EDC: N-ethyl-N′-(3-dimethyl aminopropyl)-carbodiimide hydrochloride

5.2 NHS: N-hydroxysuccinimide

5.3 LPS Se: from the Salmonella bacteria serovars enteriditis

5.4 LPS Sg: from the Salmonella bacteria serovars goldcoast

5.5. PS Sl: from the Salmonella bacteria serovars livingstone

5.6 LPS Sm: from the Salmonella bacteria serovars meleagridis

5.7 LPS St: from the Salmonella bacteria serovars typhimurium

### 6. Principle

LPS is oxidized in the presence of a protein facilitated by sodium periodate. The LPS-protein solution is desalted using a NAP-5 column. The LPS-protein complex is immobilized on a CM5-chip after activation of the carboxymethyl dextran layer-on a biosensor chip with the aid of 1-ethyl-3-(3-dimethylaminopropyl)carbodiimide hydrochloride (EDC) and N-hydroxysuccinimide (NHS) and carbohydrazide. Bound LPS is then stabilized with sodium cyanoborohydride. Prior to routine use, the performance of biosensor chip-conjugated LPS to bind anti-Salmonella antibodies is assessed using a panel of reference polyclonal agglutination sera.

### 7. Reactions

7.1. Oxidation of Carbohydrate Moiety

Periodate will induce an oxidative disruption of linkages between vicinal diols on, in particular, carbohydrate moieties as in, e.g., mannose, to yield aldehyde functionalities. This reaction is typically performed in buffers at a pH range between 4.5 and 5.5 in the dark using a freshly prepared 10 to 100 mM sodium meta-periodate in 0.1 M sodium acetate (FIG. 18).

NOTE 1. The positions of conjugations indicated in this scheme, to link up this monosaccharide within a polysaccharide as, e.g., LPS, are just examples. R′ and R indicate the distal and the proximal positions, respectively, in the carbohydrate chain. The oxidation into aldehydes may repeat itself within the polysaccharide chain in each monosaccharide constituent containing vicinal diols.

NOTE 2. Periodate will also oxidize, when present, certain β-aminoethanol derivatives such as the hydroxylysine residues in collagen, as well as methionine (to its sulfoxide) and certain thiols (usually to disulfides). In addition, N-terminal serine and threonine residues of peptides, and proteins can be selectively oxidized by periodate to aldehyde groups. These reactions, however, usually occur at a slower rate than oxidation of vicinal diols.

7.2. Conjugation to Protein

Oxidation is performed in the presence of a protein. The bis-aldehyde compounds, like the oxidized monosaccharide constituents in the polysaccharide chain of LPS here above, may react with any amino group in a protein and may form a Schiff-based linkage resulting in a substituted imine (FIG. 19).

NOTE. The substituted imine is stabilized while the complex is attached to the dextran surface by a reduction facilitated by the cyanoborohydride (reductive animation).

7.3. Immobilization to Sensor Surface

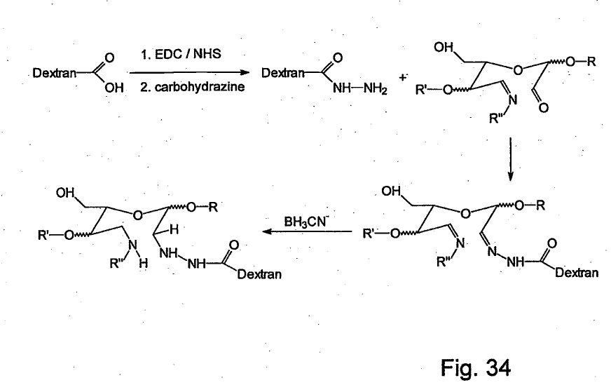

The sensorchip-located carboxymethylated dextran layer is activated by N-ethyl-N′-(3-dimethyl aminopropyl)-carbodiimide hydrochloride (EDC) and N-hydroxysuccinimide (NHS). The activation is followed by preparation with carbohydrazine. The reactive aldehyde functionalities react spontaneously with the hydrazide to hydrazones, which are then reduced to stabilize the covalent bonds (FIG. 34).

NOTE. The protein R″ carries multiple —NH₂— groups and can, therefore, be conjugated with multiple oxidized LPS entities. On the other hand, the polysaccharide part in LPS may carry multiple free aldehyde groups in a single molecule. These aldehyde groups may be, for a part or completely, captured by the hydrazide-dextran layer. The net result may be a very stable complex network of protein-LPS-dextran on the surface.

### 8. Reagents and Materials

In the complete procedure, only reagents of recognized analytical grade and only distilled water or water of equivalent purity are used, unless stated otherwise. Reference to a company is for information and identification only and does not imply a recommendation unless so stated.

8.1. Chemicals
8.1.1 Acetic acid (J. T. Baker, Deventer, The Netherlands)
8.1.2 Amine coupling kit (Biacore AB, Uppsala, Sweden) consisting of:
   8.1.2.1 Vial containing 115 mg N-hydroxysuccinimide (NHS)
   8.1.2.2 Vial containing 750 mg 1-ethyl-3-(3-dimethlylaminopropyl) carbodiimide hydrochloride (EDC).
   8.1.2.3 Vial containing 10.5 ml, c=1 mol/l, ethanolamine hydrochloride—sodium hydroxide pH 8.5
8.1.3 CHAPS (Plus one, Pharmacia Biotech, Uppsala, Sweden)
8.1.4 Carbohydrazide, CN₄H₆O (Fluka Chemie GmbH, Buchs, Switzerland)
8.1.5 Carboxymethyl-dextran sodium salt (Fluka)
8.1.6 Glycine, c=10 mmol/l pH 1.5 (Biacore)
8.1.7 Guanidine hydrochloride (Calbiochem, San Diego, Calif., USA)
8.1.8 HBS-EP buffer (Biacore) containing HEPES buffer, c=10 mmol/l, pH 7.4, sodium hydrochloride, c=150 mmol/l, EDTA, c=3 mmol/l and surfactant P20, c=0.005% (v/v)
8.1.9 Salmonella-anti-group-specific, monoclonal test reagents:
   8.1.9.1 anti-Salmonella gr. B (SIFIN, Berlin, Germany), contains mAb Anti-O4, O5, O27
   8.1.9.2 anti-Salmonella gr. C (SIFIN), contains mAb Anti-O7, O8
   8.1.9.3 anti-Salmonella gr. D (SIFIN), contains mAb Anti-O9, Vi
   8.1.9.4 anti-Salmonella gr. E (SIFIN), contains mAb Anti-O3, O19
8.1.10 Salmonella monovalent “O” somatic antisera:
   8.1.10.1 anti-O4 (Pro-Lab Diagnostics, Salmonella reference section of the Central Veterinary Laboratory, Weybridge, Great Britain)
   8.1.10.2 anti-O5 (Pro-Lab Diagnostics)
   8.1.10.3 anti-O6,7 (Pro-Lab Diagnostics)
   8.1.10.4 anti-O8 (Pro-Lab Diagnostics)
   8.1.10.5 anti-O9 (Pro-Lab Diagnostics)
   8.1.10.6 anti-O10 (Pro-Lab Diagnostics)
   8.1.10.7 anti-O12 (Pro-Lab Diagnostics)
   8.1.10.8 anti-O19 (Pro-Lab Diagnostics)
   8.1.10.9 anti-O-Poly E (O3, O10, O15, O19, O34; Pro-Lab Diagnostics)
8.1.11 Salmonella polyvalent “O” somatic antisera:
   8.1.11.1 anti-O-Poly A-S (O2, O3, O4, O5, O6,7, O8, O9, O10, O11, O12, O13, O15, O16, O17, O18, O19, O20, O21, O22, O23, O28, O30, O34, O35, O38, O40, O41; Pro-Lab Diagnostics)
8.1.12 Salmonella LPS, in-house, isolated LPS by TCA extraction (SOP CHEMIE/A21 (Example 11)) prepared from the Salmonella bacteria serovars enteriditis (Se), goldcoast (Sg), livingstone (Si), meleagridis (Sm) and typhimurium (St) with protein (SOP CHEMIE/A23, Example 13).
   8.1.12.1.1 Aliquots of 0.5 mg LPS are stored at +4° C. or at lower temperature
8.1.13 Avian reference sera
   8.1.13.1 SPF-CH, SPF serum referred to as negative control serum (Animal Health Service Ltd. (GD), Deventer, The Netherlands)
   8.1.13.2 EIA-SE, Salmonella enteritidis-positive control serum from chicken for use in ELISA (GD)
   8.1.13.3 EIA-ST, Salmonella typhimurium-positive control serum from chicken for use in ELISA (GD)
   8.1.13.4 SPA-PG, Salmonella pullorum-positive control serum from chicken for use in ELISA (GD)
   8.1.13.5 CH-SI, Salmonella infantis-positive control serum from chicken for use in ELISA (GD)
8.1.14 Sodium acetate trihydrate (J. T. Baker, Phillipsburgh, N.J., USA)
8.1.15 Sodium chloride (Merck, Darmstadt, Germany)
8.1.16 Sodium cyanoborohydride (NaCNBH₃) (Fluka)
8.1.17 Sodium hydroxide, c=50 mmol/l (Biacore)
8.1.18 Sodium periodate (Sigma Chemical Comp., St. Louis, Mo., USA)

NOTE: This chemical is light-sensitive. Store this material protected from light.
8.1.19 Triton X-100 (Sigma)
8.1.20 TWEEN®20 (Sigma)
8.1.21 TWEEN®80 (Sigma)
8.1.22 Water is obtained from a MILLI-Q® water purification system (MilliQplus)

8.2. Solutions
8.2.1 Acetic acid solution, c=0.1 g/ml: Dilute 1 ml acetic acid (8.2) with 9 ml water.
8.2.2 Acetate buffer solution, c=10 mmol/l, pH 4.0: dissolve 0.272 g sodium acetate trihydrate (8.2.14) in 180 ml water and adjust to pH 4.0 with acetic acid (8.3.1) and make up to 200 ml with water. This buffer is stable for approximately six months.
8.2.3 Acetate buffer solution, c=1.0 mol/, pH 5.5: dissolve 13.6 g sodium acetate trihydrate (8.2.14) in 90 ml water and adjust to pH 5.5 with acetic acid (8.3.1) and make up to 100 ml with water. This buffer is stable for approximately six months.
8.2.4 Acetate buffer solution, c=100 mmol/l, pH 5.5: dilute 1.0 ml acetate buffer solution, c=1.0 mol/l (8.3.3) with 9.0 ml water.
8.2.5 Carbohydrazide solution, c=100 mmol/l: dissolve 9.0 mg carbohydrazide (8.2.4) in 1000 μl water.
8.2.6 Carbohydrazide solution, c=5 mmol/l: dilute 10 μl carbohydrazide solution (8.3.5) with 190 μl water. Prepare just before use.
8.2.7 CHAPS solution, c=0.05% (m/v): dissolve 0.02 g (8.2.3) in 40 ml HBS-EP (8.2.8).
8.2.8 Detergents solution, c=0.3% (m/v): dissolve 0.3 g of CHAPS (8.2.3), 0.3 g TWEEN®20 (8.2.21), 0.3 g TWEEN®80 (8.2.22) and 0.3 g Triton X-100 (8.2.20) in 100 ml water.
8.2.9 EDC-solution: reconstitute EDC (8.1.2.2) in 10.0 ml water.
   8.2.9.1 Fractions of 100 μl of this solution (8.3.9) are stored in polypropylene tube (9.12) at −18° C. or at lower temperature. The aliquots are stable for two months.
   8.2.9.2 Before use: thaw frozen aliquots and agitate them gently to ensure homogeneous solutions.
8.2.10 Ethanolamine solution: pipette 200 μl c=1 mol/l ethanolamine solution (8.1.2.3) in a polypropylene tube (9.12).
8.2.11 Guanidine solution, c=6 mol/l: dissolve 17.18 g guanidine hydrochloride (8.2.7) in 10 ml detergents solution (8.3.8) and adjust volume to 30 ml with glycine buffer solution (8.2.6). This solution is stable for approximately three months.
8.2.12 NHS-solution: reconstitute NHS (8.1.2.1) in 10.0 ml water.
   8.2.12.1 Fractionate 100-μl aliquots of this solution (8.3.12) in polypropylene tube (9.12). Store at −18° C. or at lower temperature. The aliquots are stable for two months.
   8.2.12.2 Before use: thaw frozen aliquots and agitate them gently to ensure homogeneous solutions.
8.2.13 Sample dilution buffer: dissolve 2 g carboxymethyl-dextran sodium salt (8.2.5), 9.97 g sodium chloride (8.2.15) and 0.1 g TWEEN®80 (8.2.22) in 200 ml HBS-EP (8.2.8).
8.2.14 Sodium cyanoborohydride, c=100 mol/l: dissolve 62.8 mg sodium cyanoborohydride (8.2.16) in 1000 μl acetate solution pH 4.0 (8.3.2).
8.2.15 Sodium cyanoborohydride, c=100 mmol/l: dilute 20 μl sodium cyanoborohydride solution (8.3.14) with 180 μl acetate solution, c=10 mmol/l, pH 4.0 (8.3.2). Prepare just before use.
8.2.16 Sodium hydroxide, c=5 mmol/l: dilute 400 μl sodium hydroxide, c=50 mmol/l (8.2.17) with 3.6 ml water in glass vial (9.10).
8.2.17 Sodium periodate solution, c=100 mmol/l: add to 214 mg sodium periodate (8.2.18) 10.0 ml water.
8.2.18 Sodium periodate “ready to use”: pipette 100 μl of sodium periodate solution, c 100 mmol/l (8.3.17) in a 1.4 ml polypropylene tube (9.13) and dry with a centrifugal evaporator (8.4).
8.2.19 Sodium periodate solution, c=50 mmol/l: dissolve sodium periodate “ready to use” (8.3.18) in 200 μl acetate solution, c=100 mmol/l pH 5.5 (8.3.4). Prepare just before use.

8.3 Standard Reference Solution
8.3.1 Salmonella anti-O-sera. Dilute 20 μl of each serum (8.2.10 and 8.2.11) in 380 μl sample dilution buffer (8.3.13) in a microtiter plate (9.18) with the exception of anti-O5 serum: 2 μl of this serum (8.2.10.2) is diluted in 400 μl sample dilution buffer (8.3.13).
8.3.2 Salmonella anti-group-specific test reagents: dilute 4 μl of each serum (8.2.9.1, 8.2.9.2, 8.2.9.3 and 8.2.9.4) in 395 μl sample dilution buffer (8.3.13).
8.3.3 Avian reference sera: dilute 6 μl sera (8.2.13.1 and 8.2.13.3) in 295 μl sample dilution buffer (8.3.13) in a microtiter plate (9.18) and dilute 3 μl sera (8.2.13.2 and 8.2.13.4) in 295 μl sample dilution buffer (8.3.13).
8.3.4 Shake (9.11). Prepare just before use.

8.4 Auxiliary Materials
8.4.1 NAP-5 column (0.5 ml, Sephadex G-25, Amersham Biosciences).
8.4.2 CM5 chips (Biacore).

### 9. Equipment and Apparatus

9.1 Reference to a company is for information and identification only and does not imply a recommendation unless so stated. Equivalent equipment and apparatuses may be as appropriate as well.

9.2 Analytical balance (type AE 240, Mettler, Zurich, Switzerland)

9.3 Biacore 3000 (Biacore)

9.4 Centrifugal evaporator (Jouan, Saint-Herblain, France)

9.5 Finn pipette, 5 to 40 μl (Labsystems Oy, Helsiniki, Finland)

9.6 Finn pipette, 40 to 200 μl (Labsystems Oy)

9.7 Finn pipette, 200 to 1000 μl (Labsystems Oy)

9.8 Finn pipette, 1 to 5 ml (Labsystems Oy)

9.9 Glass collection tube, 5 ml, stoppered (Renes, Zeist, The Netherlands)

9.10 Glass vial diameter 16 mm (Biacore)

9.11 Mini shaker with microtiter cup head (MS 1, Janke & Kunkel, Staufen, Germany)

9.12 Polypropylene tube, diameter 7 mm, with snap caps (Biacore)

9.13 Polypropylene tube, 1.4 ml (Micronic, Lelystad, The Netherlands)

9.14 pH meter (type pH 537, WTW, Weilheim, Germany)

9.15 Sonification bath, Branson 2200 (Branson Ultrasonics B.V., Soest, The Netherlands)

9.16 Syringe Filter 0.45 μm diameter 25 mm (Gelman Sciences, Ann Arbor, Mich., USA)

9.17 Vacuum manifold, to run several NAP-5 columns simultaneously (type SPE-12G, with PTFE stopcock(s), J. T. Baker).

9.18 V-bottomed microtiter polystyrene plate, 96-well format (Greiner Bio-one GmbH, Frickenhausen, Germany)

9.19 Vortex-mixer (KS-1, Janke & Kunkel)

### 10. Software

The biosensor apparatus is operated with Biacore 3000 control software 4.1 (1999-2003).

### 11. Procedure

11.1 Oxidation and Desalting of LPS Solution

SAFETY PRECAUTION: Lipid polysaccharides are potent immunogens, which can bring sensitive persons into a septic shock upon intake or inhalation. Precautions should be made to prevent contact with this biochemical.

Sodium periodate is an oxidizing agent and may cause explosions when brought in contact with strong reducing agents.

The procedure utilizes sodium cyanoborohydride. The procedure should therefore be carried out with precautions, such as using hand gloves and a mask. Use this substance only in a chemical fume hood. The material is very toxic to aquatic organisms and may cause long-term adverse effect in the aquatic environment. The material and solution waste should be disposed of as hazardous waste.

11.1.1 Oxidation
11.1.1.1 Add 500 μl acetate buffer pH 5.5 (8.3.4) to the LPS (8.2.12) (see safety precaution).
11.1.1.2 Vortex (9.19) thoroughly until the pellet is dissolved.
1.1.11.3 Sonicate (9.15) the solution for 10 minutes and judge the solution for its clearance.
11.1.1.4 When clearance is not satisfactory, continue sonication until a clear (convalescent) solution is obtained.

11.1.1.5 Add 20 μl periodate solution (8.3.19) to the LPS solution (11.1.2.4).
11.1.1.6 Vortex (9.19) the solution (11.1.2.5).
11.1.1.7 Incubate on ice for 40 minutes protected from light.
11.1.1.8 Quench oxidation by desalting the solution (11.1.2.6) as described in 11.1.3.

11.1.2 Desalting
11.1.2.1 Place NAP-5 column(s) (8.5.1) on manifold (9.17).
11.1.2.2 Condition the column(s) (11.1.3.1) by passing three 3-ml portions of acetate buffer (8.3.2) over the column bed on a flow generated by gravity only. Allow the buffer to enter the gel bed completely.
1.1.2.3 Pipette 0.5 ml oxidized LPS solution (11.1.2.8) on the column. Allow the sample to enter the gel bed completely.
11.1.2.4 Elute oxidized LPS with 1 ml of acetate buffer (8.3.2). Collect eluate in a 5-ml glass tube (9.9).
11.1.2.5 Vortex (9.19) the solution (11.1.3.4) for 10 seconds.
11.1.2.6 When not immediately used (11.1.3.5), store samples at 4° C. to 7° C.
11.1.2.7 Prior to immobilization, the LPS-containing solution is diluted as indicated in the following Table 39.

TABLE 39

| LPS spp | μl stock solution | End volume (μl) make with |
|---|---|---|
| (8.2.12) | (11.1.3.6) | acetate buffer, pH 4.0 (8.3.2) |
| Se | 25 | 200 |
| Sg | 100 | 200 |
| Sl | 100 | 200 |
| St | 100 | 200 |
| Sm | 12.5 | 200 |

11.2 Immobilization

NOTE: Ensure that all solvent and reagent reservoirs contain sufficient volume to run the method completely before initiating the assay set.

NOTE: Words written in italic refer to software commands and menus, etc., operating Biacore 3000.

11.2.1 Preparation
11.2.1.1 Thaw a portion of EDC (8.3.9.1).
11.2.1.2 Thaw a portion of NHS solution (8.3.12.1).
11.2.1.3 Place the rack Thermo A in the right rack position (R2) and the Reagent rack in the middle (RR).
11.2.1.4 Command: Dock a CM5 chip (8.5.2) and prime with HBS-EP-buffer (8.2.8).
11.2.1.5 Place EDC solution (11.2.1.1) in position R2A1.
11.2.1.6 Place the NHS solution (11.2.1.2) in position R2A2.
11.2.1.7 Place an empty tube (9.12) in position R2A3.
11.2.1.8 Place the carbohydrazide solution (8.2.6) in position R2A4.
11.2.1.9 Place the ethanolamine solution (8.2.10) in position R2A5.
11.2.1.10 Place the solution with oxidized LPS (11.1.2.7) in position R2A6.
11.2.1.11 Place the cyanoborohydride solution (8.2.15) in position R2A7.
11.2.1.12 Place the 6 M guanidine solution (8.2.11) in glass vial (9.10) in position RR2.
11.2.1.13 Place the CHAPS solution (8.2.7) in glass vial (9.10) in position RR4.

PRECAUTION: Place a disposable tube under the waste outlet of the instrument to collect used cyanoborohydride solution. The solution waste should be disposed of as hazardous waste.

11.2.2 Immobilization of the CM5 chip

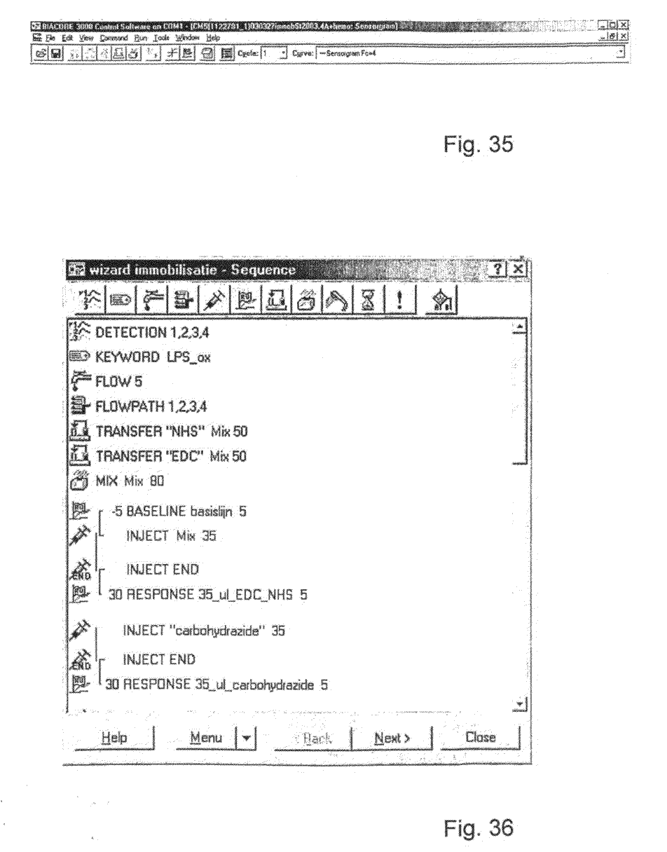

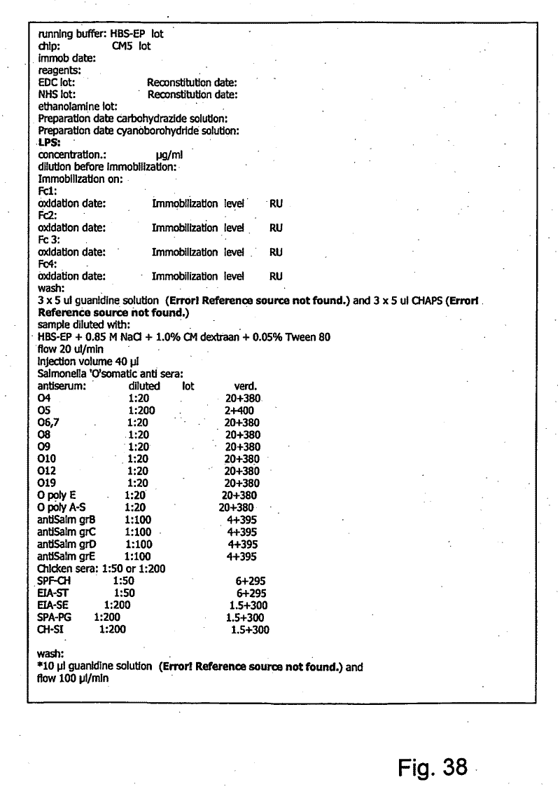

11.2.3 File (see FIG. 35.)→New application wizard
11.2.3.1 Open Template→Search for file: Wizard immobilization (see FIG. 36).
11.2.3.2 Fill in: Notebook (see FIG. 38).
11.2.3.3 Run, next and start.
11.2.3.4 Note: To see which instructions are in the wizard, do Edit instead of Run.

11.2.4 Save the sensorgrarm. The resulting files are saved with the default extension “.blr.”

11.2.5 The chip is ready for testing Salmonella antibodies in sera.

Note: The immobilization levels obtained should approach the levels as indicated in Table 40. Otherwise, consider the immobilization due to oxidation failure.

TABLE 40
Typical immobilization levels of lps.

| | Immobilization level in |
|---|---|
| LPS spp | RU (Standard deviation)¹ |
| Se | 2365 (959) |
| Sg | 8609 (1969) |
| Sl | 10953 (2135) |
| St | 4836 (1023) |

11.3 Detection of Anti-Salmonella Antibodies

Note: Ensure that all solvent and reagent reservoirs contain sufficient volume for complete method run of reference sera before initiating the assay set.

11.3.1 Make the Biacore operational with an appropriate CM5 chip.

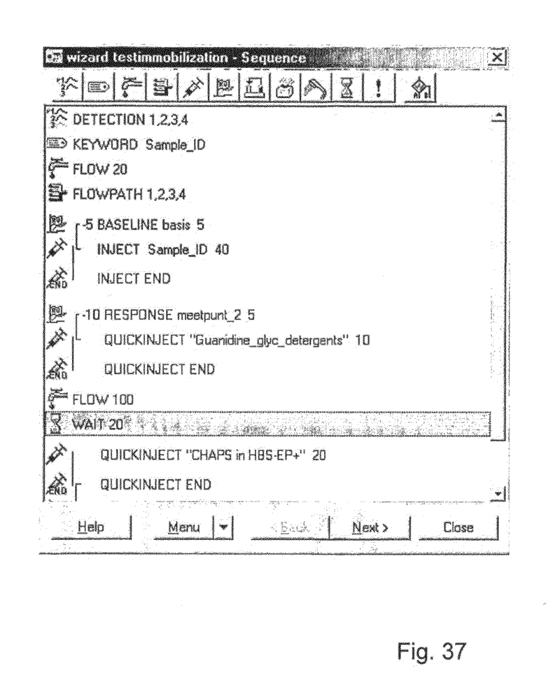

11.3.2 File (see FIG. 36)→New application wizard.
11.3.2.1 Open Template→Search for file: Wizard control chip immobilization (see FIG. 37).
11.3.2.2 Fill in: Notebook (see FIG. 38).
11.3.2.3 Run, next and start.
11.3.2.4 Note: To see which instructions are in the wizard, do Edit instead of Run.

11.3.3 Save the sensorgram. The result files are saved with the default extension “.blr.”

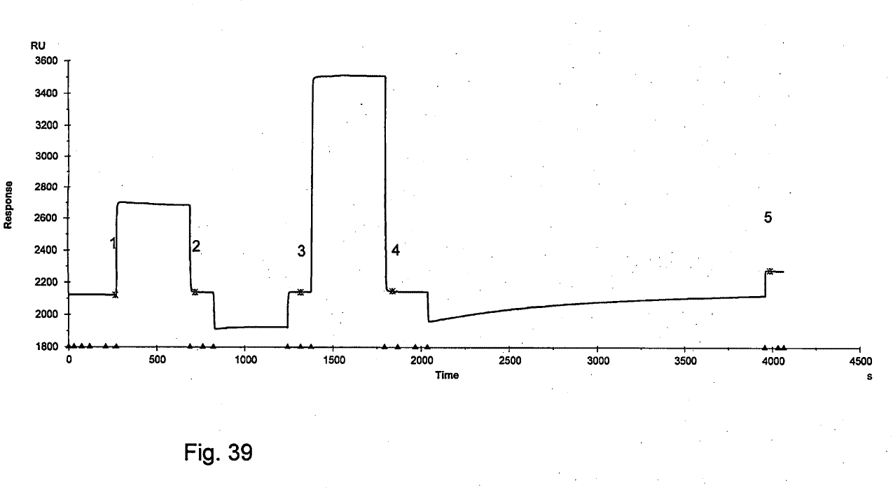

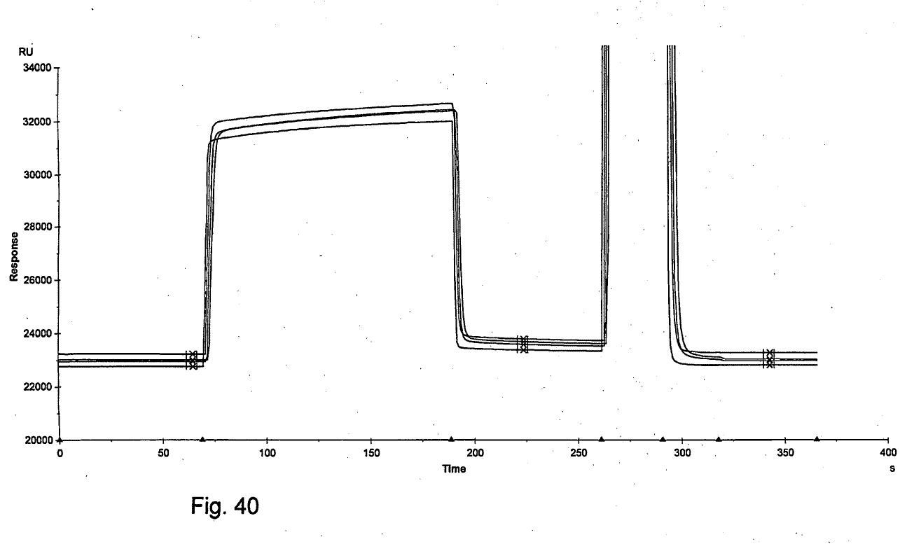

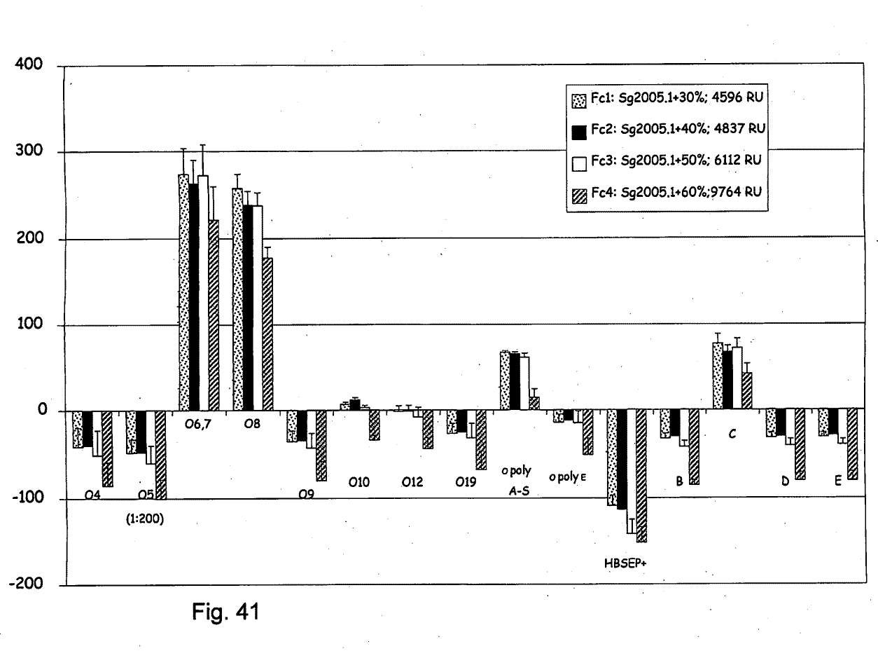

Typical sensorgram for immobilization of LPS is depicted in FIG. 39, while a typical sensorgram for the analysis of an antiserum is depicted in FIG. 40. Typical responses are listed in Table 41.

TABLE 41
Typical responses of Salmonella antibodies sera.

| | Anti Salmonella sera | | | | | | | | |
|---|---|---|---|---|---|---|---|---|---|
| | | | | | | | | | Sample |
| LPS spp | O4 | O5 | O6, 7 | O8 | O9 | O12 | O-poly E | O-poly A-S | dilution buffer |
| Se | 9 | −1 | 10 | 4 | 240 | 243 | 2 | 296 | −8 |
| Sg | 4 | −7 | 606 | 453 | −1 | 23 | 4 | 194 | −3 |
| Sl | 4 | −3 | 263 | −1 | 2 | 2 | 5 | 105 | −8 |
| St | 460 | 526 | 16 | 5 | 5 | 224 | 6 | 287 | −5 |

### Example 13

### Optimalization of Protein Addition to LPS for Immobilization and Detection of Serum Antibodies

WARNING AND SAFETY PRECAUTIONS: Lipopolysaccharides (S) are highly pyrogenic and can cause fever. To avoid intake, treat aqueous solutions of LPS with care and wear a mask when working with solid material. If any of these compounds enters the bloodstream, directly seek medical attention.

### 1. Introduction

Recent studies indicate that when protein is added to LPS before oxidation, the immobilization to a carboxymethylated dextrane gold layer is made possible and, in some cases, is improved. Following, serological responses are also made possible and are improved. The optimum of serological responses depends on the percentage of protein added to LPS.

This protein effect can be obtained by addition of hemoglobin. Hemoglobin is a naturally occurring protein which can be found in all warm-blooded vertebrates. Therefore, cross-reacting anti-hemoglobin antibodies in sera are not expected.

### 2. Scope and Field of Application

This method describes the addition of an amount of hemoglobin to lipopolysaccharides (LPS) produced through trichloric acid extraction (see SOP Chemie/A21: Extraction and isolation of Lipopolysaccharides (Example 11)). The optimal hemoglobin percentage is defined as a reaction mixture giving high immobilization levels in combination with maximum serological reaction of positive control sera. Furthermore, the production and storage of LPS reaction mixtures, ready to be used, for immobilization on sensor chips is described.

### 3. References

SOP Cheraie/A21: Extraction and isolation of Lipopolysaccharides (version 3; Example 11).

SOP CHEMIE/A22: Immobilization of Salmonella-derived LPS onto a biosensor chip (BIACORE) and detection of serum antibodies reporting a current or past Salmonella infection (version 3; Example 12).

### 4. List of Abbreviations

4.1 LPS: Lipopolysaccharides

4.2 SOP: Standard Operating Procedure

4.3 NaAc: sodium acetate buffer

4.4 Hb: Hemoglobin

4.5 mQ: Milli-Q® water

### 5. Principle

In SOP Chemie/A21 (Example 11), the production of Salmonella Spp. lipopolysaccharides (LPS) is described. Extracted LPS is used as a ligand in an analytical analysis performed on a Biacore 3000 system to detect anti-Salmonella antibodies in sera derived from pigs and chicken (see SOP CHEMIE/A22 (Example 12)). To improve immobilization of LPS, hemoglobin is added before oxidation. To produce a large stock of material to give reproducible immobilization levels, serological data and method performance, LPS is fortified with hemoglobin, divided in aliquots and dried before storage at 4° C. To immobilize a chip, one of the aliquots is batch-wise oxidized and immobilized.

### 6. Reagents and Materials

During the procedure, unless otherwise stated, use only reagents of recognized analytical grade and only distilled water or water of equivalent purity. Reference to a company is for information and identification only and does not imply a recommendation unless so stated.

6.1 Chemicals
6.1.1 Acetic acid (J. T. Baker, Deventer, The Netherlands)
6.1.2 Hemoglobin, porcine (Sigma-Aldrich, Zwijndrecht, The Netherlands)
6.1.3 Milli-Q® water (0)
6.1.4 Sodium acetate trihydrate (J. T. Baker, Phillipsburgh, N.J., USA)

6.2 Salmonella Agglutination Sera
6.2.1 anti-O4 (Pro-Lab Diagnostics, Salmonella reference section of the Central Veterinary Laboratory, Weybridge, United Kingdom)
6.2.2 anti-O5 (Pro-Lab Diagnostics)
6.2.3 anti-O6,7 (Pro-Lab Diagnostics)
6.2.4 anti-O8 (Pro-Lab Diagnostics)
6.2.5 anti-O9 (Pro-Lab Diagnostics)
6.2.6 anti-O10 (Pro-Lab Diagnostics)
6.2.7 anti-O12 (Pro-Lab Diagnostics)
6.2.8 anti-O19 (Pro-Lab Diagnostics)
6.2.9 anti-O-Poly A-S (antisera to groups A through S) (Pro-Lab Diagnostics)
6.2.10 anti-O-Poly E (antisera to factors O3, O10, O15, O19, O34) (Pro-Lab Diagnostics)

6.3 Group-Specific Salmonella Antisera
6.3.1 Enteroclon anti-Salmonella group B (Sifin, Berlin, Germany)
6.3.2 Enteroclon anti-Salmonella group C (Sifin)
6.3.3 Enteroclon anti-Salmonella group D (Sifin)
6.3.4 Enteroclon anti-Salmonella group E (Sifin)

6.4 Avian Reference Sera
6.4.1 SPF-CH, specific pathogen-free-(SPF-) negative control serum (Animal Health Service Ltd. (GD), Deventer, The Netherlands)
6.4.2 EIA-SE, chicken Salmonella enteritidis-positive control for use in ELISA (GD)
6.4.3 EIA-ST, chicken Salmonella typhimurium-positive control for use in ELISA (GD)
6.4.4 SPA-PG, chicken Salmonella pullorum-positive control for use in ELISA (GD)
.4.5 CH-SI, chicken Salmonella infantis-positive control for use in ELISA (GD)

6.5 Swine Reference Sera
6.5.1 Sw-Liv, swine Salmonella livingstone-positive control serum in ELISA (GD)
6.5.2 Sw-Typ, swine Salmonella typhimurium-positive control serum in ELISA (GD)
6.5.3 Sw-APP, swine Actinobaccilus Pleuropneumoniae-positive control serum in ELISA (GD)

6.6 Lipopolysaccharides

Lipopolysaccharides (LPS) are extracted, lyophilized and stored as described in SOP Cheznie/A21 (Example 11).
6.6.1 Salmonella enteritidis LPS
6.6.2 Salmonella goldcoast LPS
6.6.3 Salmonella livingstone LPS
6.6.4 Salmonella meleagridis LPS
6.6.5 Salmonella typhimurium LPS

6.7 Reagents
6.7.1 Acetate buffer solution, c=10 mmol/l, pH 4.0: dissolve 0.272 g sodium acetate trihydrate (6.1.4) in 180 ml MQ (6.1.3) and adjust to pH 4.0 with acetic acid (6.1.1) and make up to 200 mL with mQ (6.1.3). This buffer is stable for approximately six months at 4° C.
6.7.2 Acetate buffer solution, c=1.0 mol/l, pH 5.5: dissolve 13.6 g sodium acetate trihydrate (6.1.4) in 90 ml MQ (6.1.3) and adjust to pH 5.5 with acetic acid (6.1.1) and make up to 100 ml with mQ (6.1.3). This buffer is stable for approximately six months at 4° C.
6.7.3 Hemoglobin stock solution, 5 mg/ml: Dissolve 5 mg hemoglobin (6.1.2) in 1 ml mQ (6.1.3).

6.8 Auxiliary Materials
6.8.1 CM5 chips (Biacore AB, Uppsala, Sweden).

### 7. Equipment and Apparatus

Reference to a company is for information and identification only and does not imply a recommendation unless so stated. Equivalent equipment and apparatuses may be appropriate as well.

7.1 Centrifugal evaporator (Jouan, Saint-Herblain, France)

7.2 Biacore 3000 (Biacore)

7.3 Finn pipette, 40-200 μl (Labsystems Oy, Helsinki, Finland)

7.4 Glass collection tube, 5 ml, 12×75 mm with stop (Renes, Zeist, The Netherlands)

7.5 Milli-Q® installation (Millipore, Bedford, Mass., USA)

7.6 Sonification bath, Branson 2200 (Branson Ultrasonics, Soest, The Netherlands)

7.7 Vortex-mixer (KS-1, Janke & Kunkel, Staufen, Germany)

### 8. Procedure

8.1 Production of Stock Solution of Lipopolysaccharides
8.1.1 Collect the produced LPS (see SOP Chemie/A21 (Example 11)) from the refrigerator and let it acclimatize to room temperature.
8.1.2 Retrieve the weight of produced LPS (8.1.1) in the tube from the quality data sheet (see SOP Chemie/A21 (Example 11)).
8.1.3 Calculate the volume of mQ to be added to LPS using Formula 1 (10.1).
8.1.4 Add the calculated volume of mQ (8.1.3) to the LPS tube (8.1.2) (end-concentration LPS: 5 mg/ml).
8.1.5 Vortex thoroughly until all powder is dissolved.
8.1.6 Sonicate (7.6) the solution for 10 minutes and judge the solution for its clearance.
8.1.7 When clearance (8.1.6) is not satisfactory, continue sonication (7.6) until a clear (convalescent) solution is obtained.

8.2 Addition of Hemoglobin
8.2.1 Prepare four (one for each flow channel) LPS solution (8.17) dilutions in sodium acetate buffer with variable hemoglobin contents as described in Table 42 (9) in a glass tube (7.4).
8.2.2 The choice of relative hemoglobin starting amounts added to each newly prepared LPS extraction batch are given in Table 43 (9).
8.2.3 Fill up to a total volume of 500 μl with mQ (6.1.3) as described in Table 42 (9).

8.3 Oxidation and Desalting (see SOP Chemie/A22; Chapter 10.1 (Example 12)
8.3.1 Start oxidation from point 11.1.1.2.

8.4 Immobilization (see SOP Chemie/A22, Chapter 10.2 (Example 12)

NOTE: Immobilize oxidized LPS fortified with four different relative amounts of hemoglobin (8.3) on one CM5 chip (6.8.1) so each flow channel represents a different relative amount.

8.5 Detection of Anti-Salmonella Antibodies (see SOP Chemie/A22, Chapter 10.3 (Example 12))

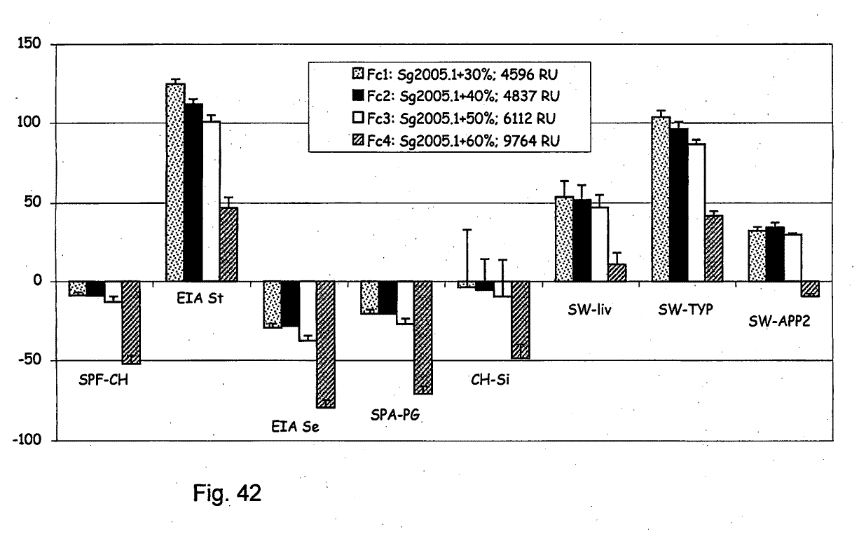

8.6 Determination of Optimal Hemoglobin Percentage
8.6.1 Calculate the mean and standard deviation of the five relative responses of each of the agglutination sera listed in (6.2) and the group-specific Salmonella antisera listed in (6.3) per flow channel.
8.6.2 Create a clustered column graph with, on the x-axis, the names of the agglutination and group-specific Salmonella antisera and, on the y-axis, the mean of the relative response units for all the different relative amounts of hemoglobin (8.6.1) (see, for example, FIG. 41).
8.6.3 Calculate the mean and standard deviation of the four relative responses of each of the avian reference control sera listed in (6.4) and the swine reference sera listed in (6.5) per flow channel.
8.6.4 Create a clustered column graph with, on the x-axis, the names of the avian control and the swine control sera and, on the y-axis, the mean of the relative response units for all the different percentages of hemoglobin (8.6.3) (see, for example, FIG. 42).
8.6.5 Add Y-error bars to both clustered graphs (8.6.2, 8.6.4) by using the standard deviation values for each x-axis column (see, for example, FIG. 41 or 42).
8.6.6 Copy the calculated means of the selected positive expected sera (see Tables 44 to 46 (9)) and the negative SPF chicken sera of all measured relative hemoglobin amounts in a new table (see, for an example, Table 47 (9).
8.6.7 Subtract the responses of SPF chicken sera (8.6.6) from the responses of the expected positive sera (8.6.6) (see, for an example, Table 48 (9)).
8.6.8 Determine the highest response per positive serum per relative hemoglobin amount and give this a value of 10 (see, for an example, Table 49 (9)).
8.6.9 Calculate for the rest of the flow channels the relative values by using Formula 2a (10.2) (see, for an example, Table 49 (9)).
8.6.10 Calculate the sum of all relative values per hemoglobin percentage (see, for an example, Table 49 (9)).
8.6.11 The optimal hemoglobin percentage (for the four percentages compared) is determined by the highest sum score in the four flow channel/hemoglobin percentages.
8.6.12 When the optimal relative hemoglobin amounts (8.6.11) is the highest or lowest hemoglobin amounts compared, steps 8.2 to 8.6.11 have to be repeated with the following conditions.
   8.6.12.1 In the case where the lowest hemoglobin amounts for Sg, Sm and Sl (20%) is the most optimal, the amounts of 20% and 30% are repeated in addition to 0% and 10% hemoglobin.
   8.6.12.2 In the case where the highest relative hemoglobin amounts for Se and St is most optimal, the amounts 20% and 30% are repeated in addition to 40% and 50% hemoglobin.
   8.6.12.3 In the case where the highest relative hemoglobin amounts for Sg, Sm and Sl is most optimal, the percentages 40% and 50% are repeated in addition to 60% and 70% hemoglobin.
8.6.13 An optimum of hemoglobin percentage is reached:
   8.6.13.1 When the sum of relative values (8.6.8) per four compared relative hemoglobin amounts has the highest value.
   8.6.13.2 In the range of compared hemoglobin where the highest value is detected, a higher and a lower level of hemoglobin addition is also determined.

8.7 Preparation of Hemoglobin Added Vacuum, Dried LPS Stock
8.7.1 The volume of the remaining 5 mg/ml LPS solution after determination of optimal hemoglobin amount is calculated by subtracting tube weight plus LPS solution (8.1.7) by the initial empty tube weight read from the quality data sheet (see SOP Chemie/A21 (Example 11).
8.7.2 The amount of remaining LPS in calculated using Formula 3 (10.3).
8.7.3 Calculate the amount of hemoglobin to be added to LPS using Formula 4 (10.4).
8.7.4 Add the calculated amount of hemoglobin (8.7.1) to the remaining LPS solution to reach an end concentration, which was determined in 8.6.13.
8.7.5 Invert, vortex and/or sonicate the solution (8.7.4) until the hemoglobin is fully dissolved.
8.7.6 Dispense 100 μl in glass tubes (7.4).
8.7.7 Dry the dispensed solution (8.7.6) in a rotating vacuum dryer (7.1) (heating point 1, 15-minutes, total run time: 60 minutes).
8.7.8 Stopper the tubes and store at 4° C. to 7° C. until further use.

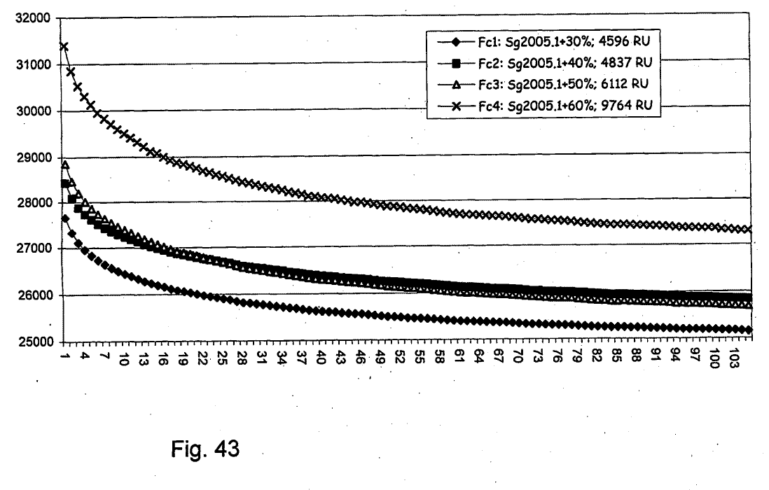

Typical baseline responses of S. goldcoast LPS hemoglobin complexes immobilized CM5 chip are given in FIG. 43.

### 9. Tables

TABLE 42
Addition of hemoglobin (6.7.3), sodium acetate buffer (6.7.2) and
mQ (6.1.3) to LPS (8.2.1) prior to oxidation.

| | Percentage hemoglobin | | | | | | | | | |
|---|---|---|---|---|---|---|---|---|---|---|
| | 0% | 10% | 20% | 30% | 40% | 50% | 60% | 70% | 80% | 90% |
| LPS (0) | 100 μl | 100 μl | 100 μl | 100 μl | 100 μl | 100 μl | 100 μl | 100 μl | 100 μl | 100 μl |
| NaAc, 1M pH | 50 μl | 50 μl | 50 μl | 50 μl | 50 μl | 50 μl | 50 μl | 50 μl | 50 μl | 50 μl |
| 5.5 (0) | | | | | | | | | | |
| Hb (5 mg/ml) (0) | 0 μl | 10 μl | 20 μl | 30 μl | 40 μl | 50 μl | 60 μl | 70 μl | 80 μl | 90 μl |
| mQ (0) | 350 μl | 340 μl | 330 μl | 320 μl | 310 μl | 300 μl | 290 μl | 280 μl | 270 μl | 260 μl |

TABLE 43
Relative hemoglobin starting amounts added to LPS.

| | |
|---|---|
| PS providing | |
| Salmonella | |
| strain | hemoglobin |

| | | | | |
|---|---|---|---|---|
| S. enteritidis (0) | 0% | 10% | 20% | 30% |
| S. goldcoast (0) | 20% | 30% | 40% | 50% |
| S. livingstone (0) | 20% | 30% | 40% | 50% |
| S. meleagridis (0) | 20% | 30% | 40% | 50% |
| S. typhimurium (0) | 0% | 10% | 20% | 30% |

TABLE 44
Expected results of (diluted) agglutination serum binding to
immobilized Salmonella LPS.

| | Agglutination sera | | | | | | | | | |
|---|---|---|---|---|---|---|---|---|---|---|
| LPS | | | | | | | | | | O-poly |
| providing | O4 | O5 | O6,7 | O8 | O9 | O10 | O12 | O19 | O-poly | E |
| Salmonella | 1:20 | 1:20 | 1:20 | 1:20 | 1:20 | 1:20 | 1:20 | 1:20 | A-S 1:20 | 1:100 |
| strain | (6.2.1) | (6.2.2) | (6.2.3) | (6.2.4) | (6.2.5) | (6.2.6) | (6.2.7) | (6.2.8) | (6.2.9) | (6.2.10) |
| S. enteritidis(6.6.1) | − | − | − | − | 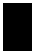 | − | 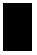 | − | 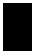 | − |
| S. goldcoast(6.6.2) | − | − | 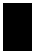 | 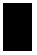 | − | − | − | − | 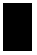 | − |
| S. livingstone(6.6.3) | − | − | 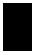 | − | − | − | − | − | 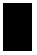 | − |
| S. meleagridis(6.6.4) | − | − | − | − | − | 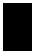 | − | − | 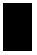 | 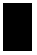 |
| S. typhimurium(6.6.5) | 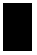 | 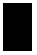 | − | − | − | − | 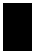 | − | 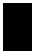 | − |

Legend:
+ = positive binding of serum to immobilized LPS
− = no binding of serum to immobilized LPS

| | |
|---|---|
| 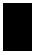 | = selected sera to be used in optimal hemoglobin addition determination |

TABLE 45
Expected results of (diluted) avian reference serum binding to
immobilized Salmonella LPS.

| | Avian reference sera | | | | |
|---|---|---|---|---|---|
| LPS providing | CH-SPF | EIA-St | EIA-Se | Spg | Si |
| Salmonella | 1:20 | 1:20 | 1:200 | 1:200 | 1:200 |
| strain | (6.4.1) | (6.4.3) | (6.4.2) | (6.4.4) | (6.4.5) |
| S. enteritidis(6.6.1) | 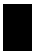 | + | 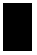 | 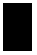 | − |
| S. goldcoast(6.6.2) | 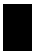 | − | − | − | + |
| S. livingstone(6.6.3) | 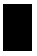 | − | − | − | 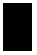 |
| S. meleagridis(6.6.4) | 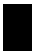 | − | − | − | − |
| S. typhimurium(6.6.5) | 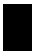 | 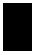 | + | + | − |

Legend:
+ = positive binding of serum to immobilized LPS
− = no binding of serum to immobilized LPS

| | |
|---|---|
| 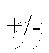 | = selected sera to be used in optimal hemoglobin addition determination |

TABLE 46
Expected results of (diluted) swine reference serum binding to
immobilized Salmonella LPS.

| | Avian reference sera | | |
|---|---|---|---|
| LPS providing | Sw-Liv | Sw-Typ | Sw-APP |
| Salmonella strain | (1:20) (6.5.1) | 1:20 (6.5.2) | 1:20 (6.5.3) |
| S. enteritidis(6.6.1) | − | + | − |
| S. goldcoast(6.6.2) | 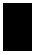 | − | − |
| S. livingstone(6.6.3) | 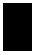 | − | − |
| S. meleagridis(6.6.4) | − | − | − |
| S. typhimurium(6.6.5) | − | 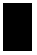 | − |

Legend:
+ = positive binding of serum to immobilized LPS
− = no binding of serum to immobilized LPS

| | |
|---|---|
| 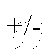 | = selected sera to be used in optimal hemoglobin addition |
| determination | |

TABLE 47
Typical serological responses on Salmonella goldcoast immobilized
LPS with variable hemoglobin additions (data of two prepared CM5 chips).

| | | | | | Anti-Salm | | |
|---|---|---|---|---|---|---|---|
| | | | | O-poly A-S | Group C | CH-SPF | Sw-Liv |
| Hemoglobin | Immobilization | O6, 7 1:20 | O8 1:20 | 1:20 | (1:100) | 1:20 | (1:20) |
| (%) | levels (RU) | (6.2.3) | (6.2.4) | (6.2.9) | (6.3.2) | (6.4.1) | (6.5.1) |
| 30 | 4596 | 273.2 | 257.1 | 66.8 | 76.8 | −9.0 | 53.7 |
| 40 | 4837 | 262.4 | 238.3 | 65.0 | 67.1 | −9.1 | 51.9 |
| 50 | 6112 | 271.9 | 237.5 | 60.9 | 71.6 | −13.1 | 47.1 |
| 60 | 9764 | 221.3 | 177.8 | 14.2 | 41.5 | −52.0 | 10.6 |
| 10 | 2177 | 179.4 | 140.0 | 39.0 | 55.0 | 0.5 | 28.0 |
| 20 | 3469 | 186.5 | 143.4 | 37.4 | 55.1 | −5.3 | 26.5 |
| 30 | 3690 | 228.3 | 186.0 | 50.1 | 80.1 | −4.7 | 35.1 |
| 40 | 5922 | 247.5 | 198.3 | 49.7 | 110.3 | −11.2 | 40.6 |

TABLE 48
Typical table of subtraction of CH-SPF from the selected positive
serological responses (data of Table 47) on Salmonella goldcoast immobilized
LPS with variable hemoglobin additions (data of two prepared CM5 chips).

| | | | | | Anti-Salm | |
|---|---|---|---|---|---|---|
| | | | | O-poly A-S | group C | Sw-Liv |
| Hemoglobin | Immobilization | O6, 7 1:20 | O8 1:20 | 1:20 | (1:100) | (1:20) |
| (%) | levels (RU) | (6.2.3) | (6.2.4) | (6.2.9) | (6.3.2) | (6.5.1) |
| 30 | 4596 | 282.2 | 266.0 | 75.8 | 85.7 | 62.6 |
| 40 | 4837 | 271.6 | 247.4 | 74.1 | 76.2 | 61.0 |
| 50 | 6112 | 284.9 | 250.6 | 74.0 | 84.6 | 60.2 |
| 60 | 9764 | 273.3 | 229.8 | 66.2 | 93.5 | 62.6 |
| 10 | 2177 | 178.9 | 139.5 | 38.5 | 54.6 | 27.5 |
| 20 | 3469 | 191.8 | 148.7 | 42.7 | 60.4 | 31.8 |
| 30 | 3690 | 233.0 | 190.7 | 54.8 | 84.8 | 39.8 |
| 40 | 5922 | 258.7 | 209.5 | 60.9 | 121.5 | 51.8 |

TABLE 49
Typical table of relative values score of Table 48 (data of two
prepared CM5 chips).

| | | | | | Anti- | | |
|---|---|---|---|---|---|---|---|
| | | | | | Salm | | |
| | | O6, 7 | | O-poly A-S | Group C | Sw-Liv | |
| Hemoglobin | Immobilization | 1:20 | O8 1:20 | 1:20 | (1:100) | (1:20) | |
| (%) | levels (RU) | (6.3.2) | (6.2.4) | (6.2.9) | (6.3.2) | (6.5.1) | sum |
| 30 | 4596 | 10 | 10 | 10 | 9 | 10 | 49 |
| 40 | 4837 | 10 | 9 | 10 | 8 | 10 | 47 |
| 50 | 6112 | 10 | 9 | 10 | 9 | 10 | 48 |
| 60 | 9764 | 10 | 9 | 9 | 10 | 10 | 47 |
| 10 | 2177 | 7 | 7 | 6 | 4 | 5 | 30 |
| 20 | 3469 | 7 | 7 | 7 | 5 | 6 | 33 |
| 30 | 3690 | 9 | 9 | 9 | 7 | 8 | 42 |
| 40 | 5922 | 10 | 10 | 10 | 10 | 10 | 50 |

### 10. Formulas

10.1 Formula 1

Calculation of volume of mQ

v=w/5
v=volume of mQ (6.1.3) (in ml)
w=weight of LPS (8.1.2) (mg) (see SOP ChemieA21 (Example 11))

10.2 Formula 2

Calculation of relative value of positive sera

Rv=Mv/Mhv*10
Rv=relative value
Mv=mean value (8.6.1)
Mhv=mean highest value (8.6.8)

10.3 Formula 3

Calculation of amount remaining LPS

z=w*0.005
Z=amount of remaining LPS (in mg)
w=weight of remaining LPS solution (8.7.1) (in mg)

10.4 Formula 4

Calculation of amount of hemoglobin

h=y*z*0.01
h=mass of hemoglobin (in mg)
y=optimal hemoglobin percentage (%) (8.6.13)
z=amount of remaining LPS (10.3) (mg)

### REFERENCES

Barrow P. (2000). Serological diagnosis of Salmonella by ELISA and other tests. In: C. Wray and A. Wray (Eds.), Salmonella in Domestic Animals. CAB International, Oxon, p. 407.
Barrow P. A., M. Desmidt, R. Ducatelle, M. Guittet, H. M. van der Heijden, P. S. Holt, J. H. Huis in't Velt, P. McDonough, K. V. Nagaraja, R. E. Porter, K. Proux, F. Sisak, C. Staak, G. Steinbach, C. J. Thorns, C. Wray, and F. van Zijderveld (1996). World Health Organization-supervised interlaboratory comparison of ELISAs for the serological detection of Salmonella enterica serotype enteritidis in chickens. Epidemiol. Infect. 117:69.
Berends B. R., F, van Knapen, D. A. Mossel, S. A. Burt, and J. M. Snijders (1998). Impact on human health of Salmonella spp. on pork in The Netherlands and the anticipated effects of some currently proposed control strategies. Int. J. Food Microbiol. 44:219.
de Vries N., K. A. Zwaagstra, J. H. Huis in't Veld, F. van Knapen, F. G. van Zijderveld, and J. G. Kusters (1998). Production of monoclonal antibodies specific for the i and 1,2 flagellar antigens of Salmonella typhimurium and characterization of their respective epitopes. Appl. Environ. Microbiol. 64:5033.
Edel W., W. J. van Leeuwen, and A. M. Hoogenboom Verdegaal (1993). The annual incidence of salmonellosis in humans in The Netherlands. Tijdschr. Diergeneeskd. 118:306.
Fratamico P. M., T. P. Strobaugh, M. B. Medina, and A. G. Gehring (1997). Real-time detection of Escherichia coli O157:H7 using a surface plasmon resonance biosensor. Book of Abstracts, 214th ACS National Meeting, Las Vegas, Nev., September 7-11, AGFD.
Grimont P., F. Grimont and P. Bouvet (2000). Taxonomy of the genus Salmonella. In: C. Wray and A. Wray (Eds.), Salmonella in Domestic Animals. CAB International, Oxon, p. 1.
Holt P. (2000). Host susceptibility, resistance and immunity to Salmonella in animals. In: C. Wray and A. Wray (Eds.), Salmonella in Domestic Animals. CAB International, Oxon, p. 73.
Hoogenboom Verdegaal A. M., J. C. de Jong, M. During, R. Hoogenveen, and J. A. Hoekstra (1994). Community-based study of the incidence of gastrointestinal diseases in The Netherlands. Epidemiol. Infect. 112:481.
Ivnitski D., I. Abdel-Hamid, P. Atanasov, and E. Wilkins (1999). Biosensors for detection of pathogenic bacteria. Biosens. Bioelectron. 14:599.
Jongerius-Gortemaker B. G. M., R. L. Goverde, F. van Knapen, and A. A. Bergwerff (2002). Surface plasmon resonance (BIACORE) detection of serum antibodies against Salmonella enteritidis and Salmonella typhimurium. J. Immunol. Meth 266:3344.
Kamerling J. P. and J. F. G. Vliegenthart (1989). Carbohydrates. In Clinical Biochemistry, Principles, Methods, Applications. Mass Spectrometry, edited by A. M. Lawson (Walter de Gruyter, Berlin), vol. 1, pp. 175-263.
Kretschmann E. and H. Raether (1968). Radiative decay of nonradiative surface plasmons excited by light. Z. Naturforsch. A. 23:2135.
Liedberg B., C. Nylander, and I. Lundstroem (1983). Surface plasmon resonance for gas detection and biosensing. Sens. Actuators 4:299.
Medina M. B. (1997). SPR biosensor: food science applications. Food Test. Anal. 3:14.
Medina M. B., L. Van Houten, P. H. Cooke, and S. I. Tu (1997). Realtime analysis of antibody binding interactions with immobilized E. coli O157:H7 cells using the BIAcore. Biotechnol. Technol. 11:173.
Popoff M. Y. (2001). In: Antigenic formulas of the Salmonella serovars, 8th revision. WHO Collaborating Centre for Reference and Research on Salmonella. Institute Pasteur, Paris, France, pp. 150.
Staub A. M. (1965). Bacterial lipido-proteino-polysaccharides (“O” somatic antigens): extraction with trichloroacetic acid. In R. L. Whistler (ed.), Methods in Carbohydrate, Chemistry, Volume V. Academic Press, New York, pp. 92-93
Thorns C. J., M. M. Bell, M. G. Sojka, and R. A. Nicholas (1996). Development and application of enzyme-linked immunosorbent assay for specific detection of Salmonella enteritidis infections in chickens based on antibodies to SEF14 fimbrial antigen. J. Clin. Microbiol. 34:792.
van Asten A. J., K. A. Zwaagstra, M. F. Baay, J. G. Kusters, J. H. Huis in't Veld, and B. A. van der Zeijst (1995). Identification of the domain that determines the g,m serotype of the flagellin of Salmonella enteritidis. J. Bacteriol. 177:1610.
Van Pelt W., A. Van de Giessen, W. Van Leeuwen, W. Wannet, A. Henken, E. Evers, M. De Wit, and Y. Van Duynhoven (1999). Oorsprong, omvang en kosten van humane salmonellose. Deel 1. Oorsprong van humane salmonellose met betrekking tot varken, rund, kip, ei eh overige bronnen. Infectieziekten Bull. 10:240.
van Pelt W., W. J. B. Wannet, A. W. van de Giessen, and Y. T. H. P. van Duynhoven (2003). Trends in gastroenteritis in the Netherlands Lowest incidences in 2002 ever; the lull before the storm? Infectieziekten Bull. 14:424.
Wilkons S. G. (1996). Bacterial lipopolysaccharides-Themes and variations. Progress in Lipid Research, volume 35, issue 3, September, p. 283-343
Yamane Y., N. Awamura, H. Fujii, H. Ohta, Y. Toyota, K. Otsuki, and T. Inoue (2000). Establishment of an enzyme-linked immunosorbent assay with a coated deflagellated. Salmonella enteritidis antigen for detection of a specific chicken antibody. Avian Dis. 44:291.

### REFERENCES IN EXAMPLE 2

Baay M. F. and J. H. Huis in't Veld (1993). Alternative antigens reduce cross-reactions in an ELISA for the detection of Salmonella enteritidis in poultry. J. Appl. Bacteriol. 74:243.
Bergwerff A. A. and F. van Knapen (2006). Surface Plasmon Resonance Biosensors for Detection of Pathogenic Micro-organisms: Strategies to Secure Food and Environmental Safety. J.A.O.A.C. Int., accepted for publication.
Bergwerff A. A. and F. van Knapen (2003). Sensing pathogens: Screening strategies in food and environmental safety. Biacore Journal 2:10-15.
Bokkers E. G. M. (2002). The Action Plan Salmonella Poultry Layer Sector, 2001+. Dutch Product Board for Livestock, Meat and Eggs. Available from http://bedrijfsnet.pve.agro.nl/pls/pbs/docs/folder/BEDRIJFSNET US CA/HOO FDTHEMAS/KWALITEIT VOEDSELVEILIGHEID/SALMONELLA CAMPYLOBACTER/ACTIEPLANNEN/EIERSECTOR/ARTIKEL ACTIEPLANSALMONELLA %20EIERSECTOR2001.PDF. In Dutch. Last visited: December 2005.
Desmidt M., R. Ducatelle, F. Haesebrouck, P. A. de'Groot, M. Verlinden, R. Wijffels, M. Hinton, J. A. Bale, and V. M. Allen (1996). Detection of antibodies to Salmonella enteritidis in sera and yolks from experimentally and naturally infected chickens. Vet. Rec. 138:223-6.
Van Duijkeren E., W. J. Wannet, D. J. Houwers, and W. van Pelt (2002). Serotype and phage type distribution of Salmonella strains isolated from humans, cattle, pigs, and chickens in the Netherlands from 1984 to 2001. J. Clin. Microbiol. 40:3980-5.
Van Duynhoven Y. T., C. M. de Jager, L. M. Kortbeek, H. Vennema, M. P. Koopmans, F. van Leusden, W. H. van der Poel, and M. J. van den Broek (2005). Explosie Project Team. A one-year intensified study of outbreaks of gastroenteritis in The Netherlands. Epidemiol. Infect. 133:9-21.
Fischer I. S. T. (2004). International trends in Salmonella serotypes 1998-2003—a surveillance report from the Enter-net international surveillance network. Euroroundup 9:9-10.
Gast R. K., M. S. Nasir, M. E. Jolley, P. S. Holt, and H. D. Stone (2002). Detection of experimental Salmonella enteritidis and S. typhimurium infections in laying hens by fluorescence polarization assay for egg yolk antibodies. Poult. Sci. 81:1128-3.1.
Gast R. K., R. E. Porter, Jr., and P. S. Holt (1997). Applying tests for specific yolk antibodies to predict contamination by Salmonella enteritidis in eggs from experimentally infected laying hens. Avian Dis. 41:195-202.
Gast R. K. and C. W. Beard (1991). Detection of Salmonella serogroup D-specific antibodies in the yolks of eggs laid by hens infected with Salmonella enteritidis. Poult. Sci. 70: 1273-6.
Guerin M. T., S. W. Martin, G. A. Darlington, and A. Rajic, A. (2005). A temporal study of Salmonella serovars in animals in Alberta between 1990 and 2001. Cam J. Vet. Res. 69:88-99.
Hassan J. O., P. A. Barrow, A. P. Mockett, and S. McLeod (1990). Antibody response to experimental Salmonella typhimurium infection in chickens measured by ELISA. Vet. Rec. 126:519.
Holt P. S., H. D. Stone, R. K. Gast, and C. R. Greene (2000). Application of the agar gel precipitin test to detect antibodies to Salmonella enterica serovar enteritidis in serum and egg yolks from infected hens. Poult. Sci. 9: 1246-50.
Jongerius-Gortemaker B. G., R. L. Goverde, F. van Knapen, and A. A. Bergwerff (2002). Surface plasmon resonance (biacore) detection of serum antibodies against Salmonella enteritidus and Salmonella typhimurium. J. Immunol. Meth. 266:33-44.
Li Z. Z., F. C. Gong, G. L. Shen, and R. Q. Yu (2002). Bacteria-modified amperometric immunosensor for a Brucella melitensis antibody assay. Anal. Sci. 18:625-30.
Liu G. D., Z. Y. Wu, S. P. Wang, G. L; Shen, and R. Q. Yu (2001). Renewable amperometric immunosensor for Schistosoma japonium antibody assay. Anal. Chem. 73:3219-26.
Metz C. E., B. A. Herman, and C. A. Roe (1998). Statistical comparison of two ROC curve estimates obtained from partially paired datasets. Med. Decis. Making 18:110-121.
Van Pelt W., A. van de Giessen, W. van Leeuwen, W. Wannet, A. Henken, E. Evers, M. de Wit, and Y. van Duynhoven (1999). Oorsprong, omvang en kosten van humane salmonellose. Deel 1. Oorsprong van humane salmonellose met betrekking tot varken, rund, kip, ei en overige bronnen. Infectieziekten Bull. 10:240-243. In Dutch.
Proux K., E. Jouy, C. Houdayer, J. Protais, M. Dibb-Fuller, E. Boscher, A. Gillard, P. Gracieux, F. Gilbert, C. Beaumont, and M. Duchet-Suchaux (2002). Reliable ELISAs showing differences between resistant and susceptible lines in hens orally inoculated with Salmonella enteritidis. Vet. Res. 33:23-33.
Pyrohova L. V., M. F. Starodub, V. P. Artiukh, L. I. Nahaieva, and H. I. Dobrosol (2002). Express diagnostics of bovine leucosis by immune sensor based on surface plasmon resonance. Ukr. Biokhim. Zh. 74:88-92. In Ukrainian.
Sachsenweger O., J. E. Lohr and J. Kosters (1994). Evaluation of three commercial ELISA test kits for the detection of antibodies against Salmonella enteritidis. Tierarztl. Prax. 22:350-7. In German.
Skov M. N., N. C. Feld, B. Carstensen, and M. Madsen (2002). The serologic response to Salmonella enteritidis and Salmonella typhimurium in experimentally infected chickens, followed by an indirect lipopolysaccharide enzyme-linked immunosorbent assay and bacteriologic examinations through a one-year period. Avian Dis. 46:265-73.
Su X., S. Low, J. Kwang, V. H. T. Chew, and S. F. Y. Li (2001). Piezoelectric quartz crystal-based veterinary diagnosis for Salmonella enteritidis infection in chicken and egg. Sensors and Actuators B: Chemical 75:29-35.
Sunwoo H. H., T. Nakano, W. T. Dixon, and J. S. Sim (1996). Immune responses in chickens against lipopolysaccharide of Escherichia coli and Salmonella typhimurium. Poult. Sci. 75:342-5.
Uttenthaler E., C. Kosslinger, and S. Drost (1998). Characterization of immobilization methods for African swine fever virus protein and antibodies with a piezoelectric immunosensor. Biosens. Bioelectron 13:1279-86′.
Vetcha S., E. Wilkins, and T. Yates (2002). Detection of hantavirus infection in hemolyzed mouse blood using alkaline phosphatase conjugate. Biosens. Bioelectron. 17:901-9.
De Vries N., K. A. Zwaagstra, J. H. Huis in't Veld, F. van Knapen, F. G. van Zijderveld, and J. G. Kusters (1998). Production of monoclonal antibodies specific for the i and 1,2 flagellar antigens of Salmonella typhimurium and characterization of their respective epitopes. Appl. Environ. Microbiol. 64:5033.
Van Zijderveld F. G., A. M. van Zijderveld-van Bemmel, and J. Anakotta (1992). Comparison of four different enzyme-linked immunosorbent assays for serological diagnosis of Salmonella enteritidis infections in experimentally infected chickens. J. Clin. Microbiol. 30:2560-6.
Zweig N. H. and G. Campbell (1993). Receiver-operating characteristic (ROC) plots: a fundamental evaluation tool in clinical medicine. Clin. Chem. 39:561-77. Erratum in: Clin. Chem. 1993, 39:1589.

### REFERENCES IN EXAMPLE 3

Benoit P. W. and D. W. Donahue (2003). Methods for rapid separation and concentration of bacteria in food that bypass time-consuming cultural enrichment. J. Food Prot. 66(10):1935-1948.
Che Y., Y. Li, and M. Slavik (2001). Detection of Campylobacter jejuni in poultry samples using an enzyme-linked immunoassay coupled with an enzyme electrode. Biosens. Bioelectron. 16(9-12):791-797.
Christensen B., H. Sommer, H. Rosenquist and N. Neilsen (2001). Risk assessment on Campylobacter jejuni in chicken product. Available Source: http://www.foodrisk.org/risk_assessments.cfm?item1=Campylobacter&item2=All+Com modities&Submit=Submit, Jun. 15, 2004.
Coker A. O. (2000). Incidence, trends and sources of Campylobacteriosis in developing countries
   An overview, pp. 44-48. In Proceedings of a WHO Consultation of Experts, Copenhagen, Denmark, 21-25 Nov. 2000. Available Source: http://www.who.int/emcdocuments/zoonoses/whocdscsraph20017c.html, Jun. 15, 2004.
FSAI (Food Safety Authority of Ireland) (2002). Control of Campylobacter species in the food chain. Available source: http://www.fsai.ie/publications/report/Campylobacter_report.pdf, Jun. 15, 2004.
Lake R., A. Hudson, P. Cressey and G. Nortje (2003). Risk Profile: Campylobacter jejuni/coli in poultry (whole and pieces). Client Report FW0109. Christchurch: ESR (Institute of Environmental Science & Research Limited). Available source: http://www.nzfsa.govt.nz/science-technology/risk profiles/campylobacter.pdf, Jun. 15, 2004.
Stem N. J. and J. E. Line (2000). Campylobacter, pp. 1040-1056. In B. M. Lund, T. C. Baird-Parker and G. W. Gould (eds.), The Microbiological Safety and Quality of Food Volume II, Aspen Publishers Inc., Gaithersburg, Md.
Tauxe R. V. (2000). Major risk factors for human Campylobacteriosis—An overview, pp. 65-66. In Proceedings of a WHO Consultation of Experts, Copenhagen, Denmark, 21-25Nov. 2000. Available Source: http://vvww.who.int/emc-documents/zoonoses/whocdscsraph20017c.html, Jun. 15, 2004.
Waller D. F. and S. A. Ogata (2000). Quantitative immunocapture PCR assay for detection of Campylobacter jejuni in foods. Appl. Environ. Microbiol. 66(9):4115-4118.
Yu L. S., J. Uknalis, and S. I. Tu (2001). Immunomagnetic separation methods for the isolation of Campylobacter jejuni from ground poultry meats. J. Immunol. Methods 256(1-2):11-18.

### REFERENCES IN EXAMPLE 4

Salinpork: Final Report of Salmonella in Pork (SALINPORK) Preharvest and harvest control options based on epidemiology, diagnostic and economic research (2000) EU project FAIR1 CT95-0400, (D. M. A. Lo Fo Wong and T. Hald, eds), Copenhagen, Denmark, pp. 251.
European Commission—Health.& Consumer Protection Directorate-General (2004). Trends and sources of zoonotic agents in animals, feeding stuffs, food and man in the European Union and Norway in 2002, Part 1, SANCO/29/2004, pp. 32.

### REFERENCES IN EXAMPLES 6, 7 and 8

Lindberg A A. (1977). Bacterial surface carbohydrates and bacteriophage adsorption. In: Surface carbohydrates of the prokaryotic cells, I. E. Sutherland, editor, London, United Kingdom: Academic Press; pp. 289-356.
Lindberg A. A. and T. Holme (1969). Influence of O side chains on the attachment of the Felix O-1 bacteriophage to Salmonella bacteria. J. Bacteriol. 99(2):513-9.
Hirsh D. C. and L. D. Martin (1983). Detection of Salmonella spp. in milk by using Felix-O1 bacteriophage and high-pressure liquid chromatography. Appl. Environs Microbiol. 46(5): 1243-5.

1. A method for immobilisation of a polysaccharide on a carrier, comprising contacting said polysaccharide with an oxidising agent and a polymer comprising at least two amine and/or amide groups to obtain a polysaccharide-polymer complex and coupling said polysaccharide-polymer complex to said carrier.

2. A method according to claim 1, said polymer is protein.

3. A method according to claim 1 or claim 2, wherein said polysaccharide is derived from a gram-negative bacterium.

4. A method according to any one of claims 1-3, wherein said polysaccharide is derived from an enterobacteriaceae.

5. A method according to any one of claims 1 to 4, wherein said polysaccharide is derived from a salmonella (sub)species.

6. A method according to any one of claims 1 to 5, wherein said polysaccharide is a lipopolysaccharide.

7. A method according to any one of claims 1 to 6, wherein said protein is haemoglobin or myoglobin.

8. A method according to any one of claims 1 to 7, wherein said oxidising agent is (sodium) m-periodate.

9. A method according to any one of claims 1 to 8, further comprising activating the surface of said carrier.

10. A method according to any one of claims 1 to 9, wherein said carrier comprises a glass surface coated with gold.

11. A method according to anyone of claims 1 to 10, wherein said carrier is modified with a coating comprising a carboxyl group donor, preferably a carboxymethylated dextran layer.

12. A method according to claim 11, wherein said carboxyl group donor and/or dextran layer is activated with 1-ethyl-3-(3-dimethylaminopropyl)carbodiimide hydrochloride, N-hydroxysuccinimide and carbohydrazide.

13. A method according to any one of claims 1 to 12, comprising at least two different polysaccharides.

14. A method according to anyone of claims 1 to 13, wherein said carrier is a biosensor chip.

15. A carrier obtained by the method according to any one of claims 1 to 14.

16. A carrier comprising an immobilised polysaccharide-protein complex on its surface.

17. A carrier according to claim 16, comprising a coating comprising a carboxyl group donor, preferably a carboxymethylated dextran, linked to a polysaccharide comprising an antigen, wherein said carboxyl group donor and said polysaccharide are linked to each other via a polymer comprising at least two amine and/or amide groups, wherein at least said polysaccharide is linked to said polymer via a periodate oxidised vincinal diol on said polysaccharide and an amine and/or amide group on said polymer.

18. A carrier according to claim 17, which is a microsphere or bead.

19. A carrier according to claim 18, wherein said microsphere or bead is a polystyrene microsphere or bead.

20. A carrier according to any one of claims 15-19, that is coded.

21. A carrier according to claim 20, wherein said carrier is coded by the presence of certain label.

22. A carrier according to claim 21, wherein said label comprises a colour.

23. A carrier according to claim 22, wherein said colour is a fluorescent or phosphorescent colour.

24. A collection of microsphere or beads comprising at least two differently encoded microsphere or beads according to any one of claims 20-23.

25. A collection of microsphere or beads according to claim 24, wherein each of said differently encoded microsphere or beads comprises a polysaccharide that comprises a different antigen.

26. A biosensor comprising a carrier according to claim 15 or 23.

27. A Surface Plasmon Resonance detection system comprising a biosensor according to claim 26.

28. A method for determining the presence of an antibody directed to an antigen of a gram-negative bacteria in a sample, comprising contacting said sample with a carrier according to any one of claims 15 or 23 or a biosensor according to claim 126 and determining whether the carrier has bound any antibody.

29. A method according to claim 28, wherein said sample is blood, blood-derived liquid material, tissue-derived fluids, such as meat drip, milk, egg, fluids from an eye, saliva or faeces.

30. A method for determining the presence of a gram-negative bacterium in a sample, comprising contacting said sample with a predetermined amount of antibodies directed against an antigen of said bacterium and determining the amount of antibodies not bound to said bacterium with a carrier according claim 15 or 23 or a biosensor according to claim 26.

31. A method according to any one of claims 28 to 30, wherein binding to said carrier or said biosensor is determined by Plasmon Surface Resonance.

32. A method according to any one of claims 28 to 31, wherein said sample is obtained from a human or an animal.

33. A method for determining the presence of a gram-negative bacterium in a sample comprising
contacting said sample with target bacteria-specific, bacteriophages and allowing the bacteriophages to infect said sample
removing non-bound and/or non-invading bacteriophages resulting in a bacteriophage infected sample
bringing the bacteriophage infected sample into contact with an indicator organism susceptible for the used bacteriophages
incubate during at least one bacteriophage multiplication cycle
recover the bacteriophages to obtain a bacteriophage-containing sample
analyse said bacteriophage-containing sample with a carrier according to claim 15 or 23 or a biosensor according to claim 26.

34. A method according to claims 33, wherein said sample is obtained from a human, a plant or an animal.

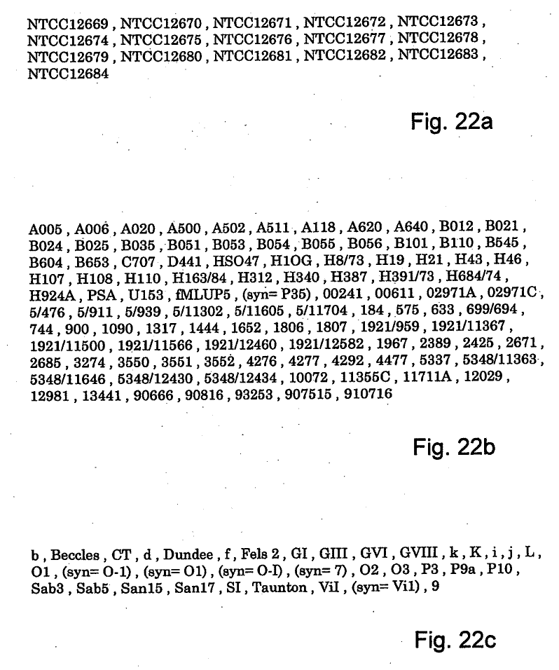

35. A method according to claim 33 and 34, wherein said bacteriophage comprises a bacteriophage of FIG. 22a, 22b and/or 22c.

36. A carrier according to any one of claims 15-23 comprising a bacteriophage of FIG. 22a, 22b and/or 22c.
